SEQUENCE LISTING (26 sequences)

SEQ ID NO: 1 - DNA, 7113 bp, Homo sapiens
gggtatataagagctggattactagttagcaaatgagggggtaaatattccagtggatacaagcttggactcttttcttgaagctttctttctatcagaagcatttgctgatattgctgacattgaaacattaaaaggtaaagaatttcctatttctgggaaagttttatttatttagagaaatgcacacttggtgttaaattcatggtttatttcaaagaaaggctaaagggagaatgtattacaatataaatgttcagattgcttagagaaggaaattgggaaagtaaaaatctcgaaattacttgaaaagtggacaatattaagggactgtatcaataaaaattttgatccttgtaaattacgttttaaaaagatgtttcttttaaaaactaagctctaatttaaaattacatcaattagaactgtaagaaatctcttgatttcagtgctggattattctttgcagaaaatttgagaagcaatgggcatcctgaagctgcaagtatttctcattgtgctctctgttgcattgaaccatctgaaagctacacccattgaaaggttggtaactttaaaatcctgtttctttgtaacttttgtaaagtgtgagaaaattagaattaaatactgtcaaataactacagccttagatttctgactatatcatacttaagaacagtaccttcagctattccattgttccttgaattctgtgttctttaaagaataacaccagtggcaaataaatatctttgatggaacttctgacagacaggaatggataattccagttttgtcaagaaatatactcttggaacttagaggggcaaagccagaacatgaagcgggaaaaaaatcaaaaggtagtaatttcttctatattaacctgatactgaaacaaaccagagaaacttaactaaagcatatatttttataccaagtggattctttttgtatatattacttagatttttgttttcctcagatgtctctggaaatgttaaaaacttttacatcttgtggaaatggaaatgtatagaaataatcaggagcaaattaatgtttttaagaaatgaaatctaaaagaagtagttaaaaagccatttgctgttgggggatttattctatattgctagctagctattctgtgagtgaaacagatttataaaaagttattcttcttattacttctaagctgctataaacttaatactttttaaaattactttcagtaggtagcatgtatgtcaggatttcctgggaagtcttattacgaaaggtttcatgtcatttaaatggtaattaaggacatctaacaactatgtcacgtaaaactcttagagtagttaaaattttcaaactgagattttaaaactgtaatttatttaaagggttattaagttcaaatatgtgcataagtcataaataacatagtgaggatttgtttgtgcctaaattagttttgctccatatagtcttatgggactgaacttacacactctttaacaccaaggagaattaagtttacctttgtaaagagtgtgcatgtcatattataattcttctcattagaatgatcgtcatcttgtctttgttttccttcgaggtagtttttcttggaagcccatagcaatatgcaaagatttctacagcacctacgtataataaatagcaagaatcattatcagaggctttttgtcatttcaaggcttatttagtttacagggtgttcttctcagaactgactgtaattttctatttgctttttcataaaaataacttttaaaatgacatgaagtttctgataagcagaatatctgaatgatgacaggaaaatcagtagtatttcctagtatatctgtttatatcttgatactttctttcaatagatatagaaatttactaagcacttttaccctctcttttttttattttattttgagacagggtctctctctctctctctctctgtctctgtcacccaggctgctggagcacagtggtacaatcatggctcactgtagccttgacattctaggctcaggtgatcctcccacctcagcctcccaagtagctgggaccacaggcacctgccaccatacccagctaattgttttatctttattttatagggaaagggtctccctatgatgcccaggctggtgtcaaactcctgggctcaagccttcctcctgcctcagtcttccaaaattctgggattataagagtgagccactgaacccagaccattatgtttttatagatgtttgtttattatgagagaaacttcacttagaaatagagcaatatgtaatataatattacttgttataaaattattttgatgttagtctcacaatctttaactttgaattattagaaatcttgtaaaacattcttcaaattgctttttaatatgttgcctgaaatgagtatgtttgaacatttgttaaagggagtatgatttgtcatgctgagatgttaaatcatgtactattctacatatctcacagaaagctaggaaaatctatggggaaaatgtgtcaaattttaaactctttttaaaaaataaaactaacattattcaatgtcattttcctcacaaaatttaatcatctcatttgagattttttcaatttgtaaatgtatgaaataggataaaaggatcacatactttcccaccaacttttttacactcccttgtaaatatctgcctggcaggtaatcaaaggatagttaaaaatataattacatagatgccaagatgcaatcactaggatctccctgcaggagctcacatacttccacagatgaatgttaaggctgagagcagggactcactttaaagtcattttgaaaactctggagagacaatttaaaagagaggcaatttaagagttatactttggcttattgtcatctctgtttaaactctcttaaagtcaagaatttccatgtgtgtatgtgcctgtaagtggtctacagctttaatgtttgttactagctcgtatgttacctgtccaggtagtcaatgagaaaaaaatgcctgaaaccagggaggtaatgccttttattaaccatttcagacaactttttccatcctaaagattgctttagatagaatcttatatatactgaatagtatatttagatgaaaagtcttttttagaaaagcaatttcacaaatatgataaaaacataaatgcttttactatttcttctaagtggaatgatggcccatctagctaactcaaataaggtaacattttatttagaacaattttaaattatattattgaccttccaaccaattatcaaaataccactcagcatttagcatataaagtatttcacactgtgcttcagtcatatgctaaacatatcttggaacagatattacctttgaatcttctcaatttgacccataattttcctttattactttttttgagatgtttggaccaaattccaatttttactgtttttcaagaaaagtaagtattttagaattcaatgcaaatgtatgaaattactagttcaatccttaaagcataaatcactcttttgaaatgtacattggtcatatttatggtaccttcaaaaataaataattgaacagatagtgtgaatgagattgatataggttaaataattagatcccaaagtggttttctttgcccagataatttgttcaaacatttgtcagcatacacttacattcaacaagtatccagttcacctaatgctgtaagaagttttctgtacttaggaagaaatatgggagtaaaatttaaaaaaaaaacagtttcacatgagattattaaatatttactcttaggctatctctacttagagatagagataatgaaatactccccacacaaggtaaacacaatgagataaatccattgcatttgagtcccagattatgcatatccactggctcctggacattgagtttttagccctataactatttcattttccatttaccctaagtttcaccaatattttgatttctatggagctgaaaactaaaacatttctctaactttcctaataatcagcaaagaggaagcaatgttattattctgcatccatttccgatatcgttttaaaagcacattgaaacaaaaggctgtcaaaaaaatagagttggtatacaaataaatgtcttaaataaaaacataagttaaaattaaatgaattatttaatgtgtggttatgatttctgagtttataagtattattatgcacttcttccaggtggctagaaaaatgtgatgaatattaataccattgacataaaaagtcttttggttttaacatttaacctagtcttatcattaaaattcttgaaagcataagatccaagcaggaaaatgtatttatgctaaaagtaataaaactctcacactgcaatagagtacctgaacaggtgatagatttgattcttttggagactttatgatattctctttttttgacatactttttatgacattattttttactttattatatttcattttattgttttaagaacaaagcatgatatctacccttttaacaaatttttaagcatgcaatacattattctggattatgtgcaaaatgttgggcagcagatctctagagcttagtcatcttgcttgactgaagctgtacacccaatggttagtaactccctatttccccctctcccttgcccctgataaccaccattccactctttaactcatgaatttgactattttaaatacttcatatacatggaaccaagtggtatttatctttctatgactagcttctttcactcaacctaatgtcctcaaggttcatccgtgtgttgcatattgcagaattcccttatgacatttcttgcataacactcctgattcaattatctcaaggaacttaaagactaagtaatgctgctttattcttattggaaagatgtagaaataattatttttaaatttcttcatatttcagattacatataaattttaccttctaaattctttttatatattaaaaataaattcttcaagatttttaaaaatgtaagacaaagacactgttattttgattatatgtaatatattctgaatttccaaaggaagacttttaactgagaaatgcaacattgactgtaatgaaagatgttgtatgattttcaattgttatttcaaggtgtcaaaaaaaaatctcagccatctaggtgtttgcaaaccaaaacactgagttacttatgtgaaaattgtttctttggttttcatcaatacaagatatttgatgtcacatggctggatccagctaaaattctaaggctctaacttttcacatttgttccatgttaccagtcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagtttgaaccttggtaaattgtaaacagctaataatgaagttattcttgacatgagaaaatcagtaattggaccaggcgcggtggctcttgcctgtaatcccagcactttgggaggccgaggcaggcagatcacaaggtcaggagttcgagaccagcctgaccaacatggtgaaaccctgtctctactaaaaatacaaaaattagccgggggtggtgacatgtgcctgtaatcccagctactcaggaggctaaggcaggagaatcgcttaaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggctatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctggaaactctttgctgtataagaattatttcttttgtttaacaaattagacatttctggcagaggttatgtatatgatacactttttttgatagcagctgcaatgttggacagaagatgaaatgctttgctttgagtcagattcttatgaatatctgcttttccctgactttgagttaggtagctttggaagtagcattaattcagataaactgccatcatgctgcgttatgccatttctaaagacactcaacttgtacttttaaaaaaatagaaaaaataagcatttcaatctaagtggaaatttgactcattgacttacatttctaagttaaaatttccctttatgaagtgtgccttaggttaccaaattgtagaggctttcgttggtggtggtaagtggtagcggtagtgagtgtatagaggcagggaaatatatttataataaattctatgtcatgaattacatattgaaataaataggtgaatatacaaatttata

SEQ ID NO: 2 - protein, 37 aa, Homo sapiens [sequence omitted]

SEQ ID NO: 3 - protein, 37 aa, Homo sapiens
KCNTATCATQRLANFLVHSGNNFGAILSSTNVGSNTY

SEQ ID NO: 4 - protein, 37 aa, Felis catus
KCNTATCATQRLANFLIRSSNNLGAILSPTNVGSNTY

SEQ ID NO: 5 - protein, 37 aa, Sus scrofa domesticus
  Synthetic oligonucleotide
KCNMATCATQHLANFLDRSRNNLGTIFSPTKVGSNTY

SEQ ID NO: 6 - protein, 26 aa, Homo sapiens
KCNTATCATQRLANFLVHSSNNFGAI

SEQ ID NO: 7 - protein, 11 aa, Homo sapiens
LSSTNVGSNTY

SEQ ID NO: 8 - protein, 13 aa, Homo sapiens
ATQRLANFLVHSS

SEQ ID NO: 9 - protein, 11 aa, Homo sapiens
TQRLANFLVHS

SEQ ID NO: 10 - protein, 21 aa, Homo sapiens
VHSSNNFGAILSSTNVGSNTY

SEQ ID NO: 11 - protein, 8 aa, Homo sapiens
TNVGSNTY

SEQ ID NO: 12 - protein, 10 aa, Homo sapiens
SNNFGAILSS

SEQ ID NO: 13 - protein, 29 aa, Homo sapiens
TQRLANFLVHSSNNFGAILSSTNVGSNTY

SEQ ID NO: 14 - protein, 67 aa, Homo sapiens
TPIESHQVEKRKCNTATCATQRLANFLVHSSNNFGAILSSTNVGSNTYGKRNAVEVLKREPLNYLPL

SEQ ID NO: 15 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 16 - protein, 140 aa, Mus musculus [sequence omitted]

SEQ ID NO: 17 - protein, 382 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 18 - protein, 320 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLA

SEQ ID NO: 19 - protein, 94 aa, Homo sapiens
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVR

SEQ ID NO: 20 - protein, 98 aa, Homo sapiens
PEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALR

SEQ ID NO: 21 - protein, 91 aa, Homo sapiens
PRVWEPVPLEEVQLVVEPEGGAVAPGGTVTLTCEVPAQPSPQIHWMKDGVPLPLPPSPVLILPEIGPQDQGTYSCVATHSSHGPQESRAVS

SEQ ID NO: 22 - DNA, 1391 bp, Homo sapiens
ggggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgcagggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttgca

SEQ ID NO: 23 - protein, 404 aa, Homo sapiens
GAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCRAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 24 - DNA, 1223 bp, Homo sapiens
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggtgaggggtttgataaagtcagggaagcagaagatagcccccaacacatgtgactggggggatggtcaacaagaaaggaatggaaggccccagaaaaccaggaggaagaggaggagcgtgcagaactgaatcagtcggaggaacctgaggcaggcgagagtagtactggagggccttgaggggcccacagacagatcccatccatcag

SEQ ID NO: 25 - DNA, 20 bp, Artificial Sequence
  misc_feature: Synthetic oligonucleotide
gctagaatggaaactgaaca

SEQ ID NO: 26 - DNA, 11299 bp, Homo sapiens
ggggcggggctggcggcgccggcgcagcccgggggcggcgggaggaggaggtggcggcggtggcgctgggagctcctgtcaccgctggggccgggccgggcgggagtgcaggggacgtgagggcgcaagggccgggacatggggcccgccagccccgctgctcgcggtctaagtcgccgcccgggccagccgccgctgccgctgctgctgccactattgctgctgcttctgcgcgcgcagcccgccatcgggagcctggccggtgggagccccggcgcggccgaggtgaggccgggccgggtcctgggggatgggggaaggggcgggaccgggtctctggacgccggcgcggacatgtccagggcagaaagcgcggtctttccagccaggtggtcagcccccaggcgcccccaatcacatttatgaacccagggttccaggccccagctcccccatcatgcgacgtcccagccccctcccatctcgagcataggaactggtctattcagagcccctggtcccagaagtccagccccctctccagacccaggtgactcggccccaaccccctcccgcctggacataggacccaccaagcagcgaggcatttagatccaataatccagaccccttgtattctctggacccatatggaggcccttgcagcctcccaggacccaggagtccagtccttcagtcaccacccaccccaaccagatgtagctctccagtcctcaaggacctggtgtccaggactgtaggcccctgaagccaggccttgtcagctttgcatcctgcaacgggagcctgagcaagggatggagggaggaggggccagaactcctgggttctggcctcctcctccgcgattcaggtttaaccccttcgggctccagagcggctgcgctggggtgggggcggagtctgtctccgcggcaacaaggcagaaagaatcccgggggacccaggtcgccatagcaacgggagcgctggggcgcccccgccctacgggagctgtttcccagggaacggtgcctccatggaggcggtgtgcggtgcttgggggagggggctggtgctgggggtctcggtcctagggagcaaagaaccaggggaccctcatgccaacgccccccgagccctcactgtcctttccacttccatccaggccccggggtcggcccaggtggctggactatgcgggcgcctaacccttcaccgggacctgcgcaccggccgctgggaaccagacccacagcgctctcgacgctgtctccgggacccgcagcgcgtgctggagtactgcagacaggtgggcggggccgaacgggagaggcggggccgcccatagaaagctagacttgaaaaaggcgtggtccagggtgctgcgcgatctaaggcgtggaggctggggggcgtggccaataaagaggcgcaactatgctaggggcaggggacctgttttgagatactaagtcaggaaaaggggagagccgcgagatagccagagaggaagtggaatttaggaatctggtggtctttgtaaagagtagaggtgtaggggggagtggcgaaaggataggcggggctaagacagaaagagaccttaaggaccagcaagatggggaaaggggtggagcccaatgagagcgcggagagctgggggggcgtggccatgaaaagacaaatttataacgggaagggagagttttggagaggcggaatagaggaaaaggcggggcctaaaggagggtgagacctttggggagacgaatctgactgcggggaggggtgaccagagaggtgggcttagagggaccttcagaaagaaacagcacaggaaaagagatagggcttaaagatgacgggacttttaagggaaaactgctagtgggcgtggccaatgagcacaaggagcttggatatctaaggctggtgctagggagaagcagggcctagggaagcgatgtcctcatgaatactagagccttgaaaacggacctggccgggcgcggtggctcacgcctgtaatcgcagcacttggggaggccgaggcaggcggatcacctgaggtcagaagttcgagaccagcctggccaacacggcgaaactccgtctctactaaaaatacaaaaattagcctggcatggtggtgcgtgcctgtaatcccagctactcaggaggctgagacaggagaatcgcttgaacctgggaggaggaggttgcagtgagccgagattgtaccattccactccagcctgggcgacaagagcaaatctccgtctcaaagaaagaaagaaagagggagaaagaaagagaaaagggacctgactactggagaggggtggctggcaggggcggggcagtgggctgattgcccccatctgatccccccagatgtacccggagctgcagattgcacgtgtggagcaggctacgcaggccatccccatggagcgctggtgcgggggttcccggagcggcagctgcgcccacccccaccaccaggttgtgcccttccgctgcctgcgtgagtcccaggcggggagaggggaactgaggtgggagtttctgaggggcaaggttctgagcccctctctcaggcctacattaaggggctgggtgcttgtgtcctaagtggggcagagaagcctctgaggataaaatatctggattctgaggagggtggggttggtggctataggaggatctcaccctggtgtcccgtgcttccccagctggtgaatttgtgagtgaggccctgctggtgcctgaaggctgccggttcttgcaccaggagcgcatggaccaatgtgagagttcaacccggaggcatcaggaggcacaggaggtcaggacgttggcccacccgtccccagcccccacaacccaggaactgggacctctaacaccctccgccaccagaaccgaggagtctgggccaccagcatcctcttcgcacttgggatctaagaatttcatcccccaaccccttcctctagaagcaggaatccaggctcccagcctcatcaacccccaaccctggcagcccagttccccatctaccccctcccatcccacaatcctggcatctgggcccactcttcctacaggcctgcagctcccagggcctcatcctgcacggctcgggcatgctcttaccctgtggctcggatcggttccgtggtgtggagtatgtgtgctgtccccctccagggacccccgacccatctgggacagcagttgggtgagtgggagggaaccctccatgcccatctcaaggttcctgaggcaggggatggaagcctgggagcccaggcctgggttcttactgcctgggtcctctcctgctccctcagtgacccctccacccggtcctggcccccggggagcagagtagagggggctgaggacgaggaagaggaggaatccttcccacagccagtagatgattacttcgtggagcctccgcaggctgaagaggaagaggaaacggtcccacccccaagctcccatacacttgcagtggtcggcaaaggtgaggcagtctctgaacccctggggcctctccaccatagagggagaaagatctgggggagtcttgctggggggtgtctttgggaggggcctataggggaaaggcccaactgaggagaaaagacgagagtatctttggataaaatagaagtagaagggctaacctgccaagggagggggtggtttgggggtacttgggagtagaggggccattgggtaggtcttgaggatcatttcaggaaagcttggaagatggtgtaatggattcctaagctttgcaagaacaggcccagtccagaactacatctcccataatgccaggcagcagcggtggctaaactgggtgcatgatggtctccagtgcactctaggaaatgtggttctctaggtagaaaaggcgacctggaggtgggctgcagactgacctcctgatccctggtcttgcagtcactcccaccccgaggcccacagacggtgtggatatttactttggcatgcctggggaaatcagtgagcacgaggggttcctgagggccaagatggacctggaggagcgtaggatgcgccagattaatgaggtgataatactgggggccccaggaccccctacagtacagagctccctaaataccaggaaattcctccaggacacattgatactacctccaaaggctccctaagcccctttgaccttgagctctcaacaccaccccctaagatggccagagatccatggcccttctagaatcccactgagacgctaccaggttctctggaaactctggtctatggtactctttcactttattggtttttttttttttcttttgttgttgttgttgtgacggagtttcgctcttaacacccaggctggagtgcaatggtgcgatctcggcccactgcaacctctgcctcccgggctccagcgattccccttcctcagtctcctgagtagctgggattacaggcacccaccaccacgcccggctaatttttgtatttttagtagagacagggtttcaccatgttggccaggatggtcttgaactcccggcgggaggagatccacccgcctcggcctcccaaagtgctgggattacaggcatgagccaccacgcctggcctctctttcactttaaactccttctggatcttccctcttgggaacccaggagccagcgagacttaagggatctggggcctttaaatctttttttttttttttttttgagacagagtttcgctctgttgcccaggctagagtgcagtgacgtgatctcccactcactgcaagctccacctcctgggttcacgccattctcctgcctcagcctcccgagtagctgggactactggtacccaccacagcgcccagataattttttctgttttcagtagagacagggtttcaccatgttagccaggatggcctcaatctcctgaccttgtgatccacccacctcggcctcccaaagtgctgggattacaggcatgagccactgcgcccagccatggggcttctaaaatcttaaagaggggttgggggacttgccaggtggatcagggtggattctgggatcctgaagctcccctccctatgcaggtgatgcgtgaatgggccatggcagacaaccagtccaagaacctgcctaaagccgacagacaggccctgaatgaggtaggacagccccagtgggtcctactcatgcctgtccaccacctggagcacactcagtttcacctggctctggctgtgccctgcccatccagttccaccccttcccacctatctcagcctttcctggccccatgcctacatgcagctctgcccctcttagccgtcatctgacctgacactgctctcctccccagattggccatattcggccccatctacagacttgacttgcctctcagggctggctctggagtcctgtcccaagccagggcctctgcagatgcagccagggccttcttggtctctctttgatgcatttatgtctctatcaggccccgccccctgattctggctctgctgggccaatctcacctttattaacctgacctaccccatggagaccccactcatgttagcccccattccagctctttgtcccacccctatcgtgtcatttatacacagcctgtctccagtttgaccctgcccaggccaggagccctgcaaggctttgtccctttcaccttaacattggtcagttctgctcccagattgctcccactcaatcttacagtttacatcctcacattggctcccagtgggcctagtcccacctccactctgcctggccctgtagcccaccccttccagtccataacctttggttctgcccaggcctggacccctggaacgccccccaaccccatgtagccctgcctttccaggctctctttgaccaggctttgacccatcttctcctctcctgaccctgtgcccacccgctccccagcacttccagtccattctgcagactctggaggagcaggtgtctggtgagcgacagcgcctggtggaaacccacgccacccgcgtcatcgcccttatcaacgaccagcgccgggctgccttggagggcttcctggcagccctgcaggcagatccgcctcaggtgcggggaccgtgggggcagagagcagagggtgagaagggtcagggcgggcttgggcatcctgtgtcccttccacaggcggagcgtgtcctgttggccctgcggcgctacctgcgtgcggagcagaaggaacagaggcacacgctgcgccactaccagcatgtggccgccgtggatcccgagaaggcacagcagatgcgcttccaggtgctcacatccttccagctcccaaatgcgccgctattcctcagacgcccgcgcctcaggctcttctcttgtcccttagaccctctttctgtctcttggaccccttcctatcccctgaacaccgcttctctgccccttcccagtctctcagctcagcttcctgaccctgaaacatggaccctcacatgctgtgtctttgacccctgcttcttggcccttggattcctactccccccgccgtcgatcctatgttctgtcccttggattttcactgcctttcccagaatcgtcttttttttttttttttttttgagacaggttcttgctctgtcgcccaggcaggagagcagtgtgcgatcttggctcattgcaacttccacctcctgggttcaagcaattctcctgcctcagcctctcgagtagctgggattacaggagcctgccaccacactgggctaatttttttttttttttttgacagagtctcgctctgtttcccaggctggagtgcagtgacatgatctgggctcactgcaacctccgcctactgggttcaagctattctcctgcctcagcctcctgagtagctgggactacaggcgggtgtcaccacatctggctgatttttgtatttttagtagagacagggtttcaccatactggtcaggctggtcttgaactcgacctcaggtgatccacccttggcctcctaaagtactcggattacaggtgtgagccaccacgcccggccccagctaatttttgtatttttggtagacacgggtttcagcatgttggccaggctggtcttgaactcctgacctcaggtgatctgcctgccttggcctcccaaagtgctgggattacaggcgtgagccaccatgcccagccagaaaccccaataacttttgcaccaatctaatatttttagcagagacagggttttgccatgttgcccaggctggtctcgaactcctgacctcaggtgatctgcccacctcggcctcccaaagtgctgggattacaggcgtgagccaccatgcccggccagaaaccccaataacttgcaccaatctaatatttttagcagagacagggttttgccatgttgcccaggctagtctcaaactcctgacctcaggtgatctgcctacctcggcctcccaaagtgctgggattacaggcatgagccaccgcgcccggtcgagaatctccttcttgttccttgaaccctcttcctgtccctcaacctcctttctccataacttcacttgttttccctggaacccctgttctgtgcgctcaaatttgaattcccctttcctggatgttttcttcctgtctatgaaactccattctgtgctcttgaactccaaatcttgccttgaaccatgtcatttctatatgaccctccaatcctcaatctctgtctctggaatcccctcaaaccccactttctgttccttggactttattcttcaatttccttctcctatggcccagttcctaacccttgtaccacacatcctgtccattgcatgtgccgcttttcctcagtcgctattgaattcctccttcatactgcttcagtttcctcatctccagcctgcattgcgcagttcatccttcatgtccactcacccacaggtgcatacccaccttcaagtgattgaggagagggtgaatcagagcctgggcctgcttgaccagaacccccacctggctcaggagctgcggccccaaatccgtgagtgtctattaccctggctcccattacagatctctgagggcagatcttgactcctaaatgttgggcccccccaatttcatttattcctctataacaaacagcccagaccttagcagtgaaaatcaacaatgatttttctttgttcatgattctgccatccggtctgcgctcagcagagtggttctttcagtggtcttgccagtggtcaagcatgcagctgtatttagctagcagatcatctaggggctgggagtctagcacaaatggacctttctctctctccaaggaagcgcaaggcctctcttctccgtggagcttctccatgtggtctcatcagcagggtagctagattccctacatggtggtttatgctctctaagacatcacagtggaagttgctaggtcttaaggcttgggcccacattctatttgttaaagcaagttacaaattcagtccagattcaagggaaggaacctatatgcataccggaaagtgtgacctattgcagcccccacatctattgtgtctttctcctggatatctcacacataaccctgattctcctagtatttaagaaagctatcatcttgaggcgcggtggctcacgcctataatcccagcactttaggaggccgaggcgggtggatcacttgaggtcaggagttcgagaccagcctggccaacatggtgaaaccccgtctttactaaaaatacaaaaatcagccgggcatgatgtcgcttgcctgtaatcccagctacttaggaggctgaggcaagagaattgcttgaacccgggaggtggaggttgcagtgagctgagatcgcatcattgcactccagctgggcaacaagagtgagactctgtctcaaaaaaaaaaaaacaaaaaaaaaacataatcttgaaacttcagcctccatccttcctgccagcagtgcctccatccagcttcccactttctcagatcacacttctggctaccccacacttggggctgactctgctgtctgcatgatctcccacttgctctactggtagggtgccctccactcacccctatgctcactacctcagccacctttctgcatgtccccctcagaggaactcctccactctgaacacctgggtcccagtgaattggaagcccctgcccctgggggcagcagcgaggacaagggtgggctgcagcctccagattccaaggatggtgagtgagcccacatatagatgaccccagacattagggaacaggccccagcctaatttgtaatcccctagagtctgagggtgtcttcaccaccacagtgactgggagaggatgaggaggaacgtctaaggttgcaggggcctctgtaggatccccaatcctccttcttagtccctggaaggatgtttctccacctttctttgctgataccctcctctcttcactgttccactcccttgcttcctctggctgccagcagacacccccatgacccttccaaaaggtgagtgtctcacagttaaccccagcctccaaatcccactgaatccctgaacccagaaggaaacagggtccatccattgggaacctcagaccccctggggtagagtttgatgtactttccagccccctcctctggaccctaaagaatgagatagggccaggcgctggtgactcacacccgtaatcctagcactttcagaggctgaggcaggaggatcccttgaggccacgagttctagaccagcctgggcaacataatgagaccctgtacctacaaataatttaaaaattacctgggtgtggtggggcatgtctgtagtcccagctgctcaggaggctgacgtagaaggatcactggagcccaggaagttgaggctgcagtgagctgagatcatgccactgcactccagcctgggtgacagagtgagactctgtctaaagaaaaaaaaaaagaatgagatcagacttgggggtagggtccacagaacaagatgctgcatcccctgagaaagagaagatgaacccgctggaacagtatgagcgaaaggtaagttagtcagaactgtgggctccctaaggggaacaagatcggggcctatatggctgggtacgagggaggagatgctgggggcttggattccttgtcctgagggaagagggagctgaggacgtggaattgagatcctagaaaatgagagggctgggggacgctctcttgggcccttgggtaggaagaagccagtgccaggcttctgggttcctgacacctcctgctcccccaggtgaatgcgtctgttccaaggggtttccctttccactcatcggagattcagagggatgagctggtaagaggaggaacagccgggtacctaggggaagagaccagaggtcagcggccaggctgtgattcccaaagccacacaggaccctcaaagaagccctctgccccatctcctctccctgcaggcaccagctgggacaggggtgtcccgtgaggctgtgtcgggtctgctgatcatgggagcgggcggaggctccctcatcgtcctctccatgctgctcctgcgcaggaagaagccctacggggctatcagccatggcgtggtggaggtgagaaccatggcgtggtggaggtgtgggaagagttcctgagcccgggtgtgggcggcctgagagacttgcgggcagtcccgcccccgcaccacactgtcctttccctcccctgctcgttgcaggtggaccccatgctgaccctggaggagcagcagctccgcgaactgcagcggcacggctatgagaaccccacttaccgcttcctggaggaacgaccctgacccggcccccttcaccccttcagccgagcccagacctcccctcttcctggagccccagaaccccaactcccagcctagggcagcagggagtcttgaagtgatcatttcacacccttttgtgagacggctggaaattcttatttcccctttccaattccaaaattccatccctaagaattcccagatagtcccagcagcctccccacgtggcacctcctcaccttaatttattttttaagtttatttatggctctttaaggtgaccgccaccttggtcctagtgtctattccctggaattcaccctctcatgtttccctactaacatcccaataaagtcctcttccctaccaggcca

(19) United States
(12) Patent Application Publication
(10) Pub. No.: US 2013/0022620 A1
(43) Pub. Date: Jan. 24, 2013

(54) ASSAYS AND METHODS PERTAINING TO PRE-AMYLOID INTERMEDIATES

(71) Applicants: Ann Marie Schmidt, Franklin Lakes, NJ (US); Andisheh Abedini, New York, NY (US); Daniel Raleigh, Stony Brook, NY (US)

(72) Inventors: Ann Marie Schmidt, Franklin Lakes, NJ (US); Andisheh Abedini, New York, NY (US); Daniel Raleigh, Stony Brook, NY (US)

(21) Appl. No.: 13/493,502

(22) Filed: Jun. 11, 2012

Related U.S. Application Data
(60) Provisional application No. 61/520,396, filed on Jun. 9, 2011.

(51) Int. Cl. G01N 33/566; C12N 5/071; C12N 15/85; C40B 30/04; A61K 39/395; A61P 1/18; A61K 35/39; A61K 38/17; A61P 3/10; A61P 25/28; A61P 25/16; C12Q 1/18; A61K 48/00

(52) U.S. Cl. 424/172.1; 436/501; 435/32; 435/375; 435/455; 506/9; 424/93.21; 424/93.7; 514/1.1; 514/6.9; 514/17.8; 514/17.7

(57) ABSTRACT

The present invention relates to amyloidogenic peptides, polypeptides and proteins; and methods for screening to identify modulators of polypeptide self-aggregation into amyloids. The invention further relates to assays and methods using islet amyloid polypeptide (IAPP) as a component of a model system with which to screen for modulators of islet amyloid formation and accumulation. Also encompassed are modulators identified using the assays and methods described herein and compositions comprising same. The present invention also relates to methods and compositions for modulating amyloid formation and accumulation, thereby providing novel treatments for amyloidoses. In a particular aspect, methods and compositions are presented for inhibiting islet amyloid formation and accumulation, thereby providing novel treatments for diabetes.

### CROSS REFERENCE TO RELATED APPLICATIONS

This application claims priority under 35 USC §119(e) from U.S. Provisional Application Ser. No. 61/520,396, filed Jun. 9, 2011, which application is herein specifically incorporated by reference in its entirety.

### GOVERNMENT RIGHTS

The research leading to the present invention was funded in part by grants F32DK089734-01 and GM078114 from the National Institute of Health. The United States government has certain rights in the invention.

### FIELD OF THE INVENTION

The present invention relates to amyloid-forming peptides, polypeptides and proteins, and more particularly, to islet amyloid polypeptide (IAPP, also known as amylin), the pro form of IAPP and processing intermediates of pro-IAPP. The invention further relates to assays and methods for screening to identify modulators of amyloidogenic peptide, polypeptide and protein aggregates. More particularly, the invention relates to assays and methods using IAPP as a component of a model system with which to screen for modulators of islet amyloid formation and accumulation. Modulators identified using the assays and methods described herein may inhibit or promote islet amyloid formation and accumulation. Also encompassed are modulators identified using the assays and methods described herein and compositions comprising same. The present invention also relates to methods and compositions for modulating amyloid formation and accumulation, thereby providing novel treatments for diseases associated with protein misfolding. In a particular aspect, methods and compositions are presented for inhibiting islet amyloid formation and accumulation, thereby providing novel treatments for diabetes.

### BACKGROUND OF THE INVENTION

A wide range of human diseases result from the inability of specific polypeptides and proteins to fold into their correct biologically active three dimensional structures, or from the failure of proteins to remain in their properly folded states [Chiti et al. (2006) Annu Rev Biochem 75, 333-366; Sipe et al. (1994) Crit. Rev Clin Lab Sci 31, 325-354; Selkoe. (2004) Nature Cell Biol 6, 1054-1061; Jahn et al. (2008) Arch Biochem Biophys 469, 100-117]. These protein misfolding diseases result from a variety of causes. In some cases, the efficiency of folding may be compromised by a range of post-translational events leading to insufficient production of active proteins; however the majority of protein misfolding diseases are caused by the transformation of normally soluble proteins or polypeptides into ordered aggregates. The latter diseases are commonly referred to broadly as “amyloidoses”. They represent a large group of diseases characterized by the deposition of insoluble ordered protein deposits that are known as amyloid fibrils or amyloid plaques. The term amyloid is used to refer to a specific type of protein quaternary cross-β structure resulting from the self-assembly of peptides, polypeptides and proteins into ordered aggregates.

Amyloid deposition is the pathological marker of many prevalent human diseases. The process of pancreatic islet amyloid formation and accumulation accelerates the decline of insulin production and secretion in type 2 diabetes (T2D), and leads to islet cell transplant failure during treatment of type 1 diabetes (T1D). Islet amyloidosis in T2D results from the dense aggregation of islet amyloid polypeptide (IAPP) in the pancreas. The mechanism of IAPP toxicity is, however, not known, despite its obvious importance.

The citation of references herein shall not be construed as an admission that such is prior art to the present invention.

Several publications and patent documents are referenced in this application in order to more fully describe the state of the art to which this invention pertains. The disclosure of each of these publications and documents is incorporated by reference herein.

Other features and advantages of the invention will be apparent from the detailed description, the drawings, and the claims.

### SUMMARY OF THE INVENTION

Amyloid deposition plays an important role in the pathology of many human diseases including type 2 diabetes (T2D) and Alzheimer's disease (AD). The process of amyloid formation is cytotoxic and contributes to the severity of disease. Despite an appreciation of this link, the mechanism(s) of toxicity are not completely understood. To better understand the molecular mechanisms of cellular toxicity in amyloidosis, the present inventors employed islet amyloid polypeptide (IAPP, also know as amylin) as a model system. Islet amyloidosis in T2D results from the dense aggregation of IAPP in the pancreas. The process of amyloid formation by IAPP leads to the dysfunction and death of pancreatic insulin-producing β-cells during T2D, as well as to islet transplant failure during treatment of T1D. As described herein, the present inventors directly show that transient, pre-fibrillar oligomers that form early in the amyloid formation process are the toxic species. Experiments which alter the time course of amyloid formation reveal that the time points of maximum toxicity correlate with the midpoint of the lag phase, suggesting that toxic species may be on-pathway to amyloid formation. Some inhibitors of amyloid formation which prolong the lag phase, extend the time course of toxicity. Biophysical characterization of the toxic intermediates indicates that these species are soluble, do not bind 1-anilino-8-naphthalene sulfonate (ANS), and lack significant β-sheet structure.

The present inventors, furthermore, show that toxic intermediates of IAPP are ligands of the receptor for advanced glycation endproducts (RAGE). RAGE is a multi-ligand receptor of the immunoglobulin superfamily that is expressed in amyloid-rich environments, and is up-regulated in inflammatory disorders such as diabetes. RAGE activates signaling cascades involved in cellular stress responses, including pro-inflammatory cytokine production and apoptosis. Neurotoxic amyloid-β (Aβ) peptides bind to RAGE, and RAGE activation in the brain of individuals with AD has been shown to lead to neurological dysfunction. Given the similar polypeptide sequences and aggregation kinetics of human IAPP and Aβ, the present inventors hypothesized that activation of RAGE by human IAPP binding may be a mechanism of islet amyloid toxicity in T2D. Results presented herein, moreover, reveal for the first time that transient, toxic intermediates of IAPP induce up-regulation of MCP-1 and IL-β mRNA, and trigger apoptosis in rat INS-1 β-cells and mouse smooth muscle cells. Additional studies presented herein indicate that the variable (V)-type domain of RAGE is important for RAGE/IAPP recognition. Further analyses reveal that competitive inhibitors of IAPP binding to RAGE [e.g., soluble RAGE (sRAGE) or an anti-RAGE antibody described herein) inhibit both IAPP amyloid formation and cytotoxicity. In light of the above, findings set forth herein demonstrate a nexus between RAGE and engagement thereof and IAPP toxicity, and suggest a role for RAGE in islet amyloid toxicity in T2D. These results have implications for the treatment of islet amyloidosis in T2D; and may be applicable to the prevention and treatment of other amyloidosis diseases, as common structures and mechanisms of toxicity have been proposed for pathological amyloidogenic species derived from different peptides, polypeptides and proteins.

In accordance with the present findings and in a first aspect, a method for screening to identify an inhibitor of amyloidogenic polypeptide self-aggregation into amyloids is presented herein, the method comprising the steps of (a) providing an amyloidogenic polypeptide under conditions that permit self-assembly and adding a candidate agent thereto, wherein the candidate agent is added to the polypeptide during lag phase of amyloid formation of the polypeptide, wherein oligomeric precursors of mature amyloid fibrils are formed and (b) detecting the degree of oligomerization of the amyloidogenic polypeptide at equilibrium in the presence of the candidate agent and comparing that to the degree of oligomerization of the amyloidogenic polypeptide at equilibrium in the absence of the candidate agent, wherein a reduction in the degree of oligomerization of the amyloidogenic polypeptide at equilibrium in the presence of the candidate agent relative to that detected in the absence of the candidate agent indicates that the candidate agent is an inhibitor of amyloidogenic polypeptide self-aggregation into amyloids. The method may further comprise measuring binding of the candidate agent to the oligomeric precursors during the lag phase to detect complexes comprising the candidate agent bound to the oligomeric precursors.

In a second aspect, a method of screening for a prophylactic and/or therapeutic agent useful in the prophylaxis and/or treatment of a subject afflicted with an amyloidoses is presented, the method comprising the steps of (a) providing an amyloidogenic polypeptide under conditions that permit self-assembly and adding a candidate agent thereto, wherein the candidate agent is added to the amyloidogenic polypeptide during the lag phase of amyloid formation, wherein oligomeric precursors of mature amyloid fibrils are formed and (b) detecting the degree of oligomerization of the amyloidogenic polypeptide at equilibrium in the presence of the candidate agent and comparing that to the degree of oligomerization of the amyloidogenic polypeptide at equilibrium in the absence of the candidate agent, wherein a reduction in the degree of oligomerization of the amyloidogenic polypeptide at equilibrium in the presence of the candidate agent relative to that detected in the absence of the candidate agent indicates that the candidate agent inhibits oligomerization of the amyloidogenic polypeptide into amyloid, and the identification of a candidate agent that inhibits oligomerization of the amyloidogenic polypeptide into amyloid indicates that the candidate agent is the prophylactic and/or therapeutic agent useful in the prophylaxis and/or treatment of a subject afflicted with an amyloidoses. The method may further comprise measuring binding of the candidate agent to the oligomeric precursors during the lag phase to detect complexes comprising the candidate agent bound to the oligomeric precursors. In an aspect thereof, the amyloidoses is any disorder in which amyloid formation causes cell death, organ failure or disease. More particularly, the amyloidoses is diabetes, Alzheimer's Disease (AD), or Parkinson's Disease (PD).

As described herein, the present methods directed to identifying modulators of polypeptide self-assembly to form amyloids or prophylactic and/or therapeutic agents (e.g., an inhibitor of amyloidogenic polypeptide self-aggregation) can utilize any peptide, polypeptide or protein that is capable of undergoing self-assembly. Peptides, polypeptides and proteins capable of self-aggregation to form amyloid fibrils and plaques are referred to herein as amyloidogenic polypeptides. Polypeptides that self-aggregate to form amyloids that are associated with amyloidosis diseases are of particular interest with respect to the present methods. Such amyloidogenic polypeptides include, but are not limited to, those listed in Table 1. Exemplary amyloidogenic polypeptides include: human islet amyloid polypeptide (IAPP), amyloid-β (Aβ), α-synuclein and tau.

In accordance with the present methods, the lag phase during amyloid formation varies, depending on a variety of parameters known to those skilled in the art. Such parameters include, but are not limited to the amyloidogenic polypeptide assayed, protein concentration, temperature, pH, pressure, ionic strength, agitation/stirring, and the presence or absence of inhibitors or catalysts (i.e. solvents, proteins and/or small molecules) that alter the rate of the nucleation and/or polymerization reactions. The kinetics of amyloid formation typically exhibits a sigmoidal polymerization (or fibrillization) profile consisting of three observable phases: the lag phase, the growth phase (or elongation phase) and the saturation phase (FIG. 1). In the lag phase, oligomeric nuclei are formed in a slow process that involves unfavorable intermolecular interactions of polypeptide monomers, wherein little or no amyloid is formed.

With respect to the oligomeric precursors of human IAPP described herein, which are identified as toxic oligomers of human IAPP, such toxic oligomers are formed during the lag phase and are soluble and cannot be pelleted by centrifugation at 25,000 G for 25 minutes. Additional biophysical properties of toxic oligomers of human IAPP are as follows: they are not molten globules and, moreover, lack detectable beta sheet character (FIG. 4). Although not wishing to be bound by theory, the toxic oligomers of IAPP could be, but are not limited to, oligomers with two or more IAPP monomers per oligomer.

One skilled in the art could readily apply the biophysical properties of IAPP oligomeric precursors as described herein to the assessment of oligomeric precursors of other amyloidogenic polypeptides and thus approximate and determine lag phase for other amyloidogenic polypeptides. Exemplary conditions conducive to formation of fibrils with respect to IAPP as described herein thus serve as a reasonable starting point for assessment of other amyloidogenic polypeptides, determination of lag phase of amyloid formation, and evaluation of oligomeric precursors formed during lag phase.

It is understood that the time course of amyloid formation is different for different proteins under the same conditions, and different for the same protein under different conditions. Accordingly, methods encompassed herein are performed wherein the lag phase of amyloid formation is between about 0-500 hours after the amyloidogenic polypeptide is provided under conditions that permit self-assembly. More particularly, the lag phase is between about 0-350 hours and even more particularly, the lag phase is between about 0-100 hours. In a further embodiment of methods encompassed herein, equilibrium is reached after 40-1000 hours after dissolution of the amyloidogenic polypeptide as provided under conditions that permit self-assembly. In a more particular embodiment, equilibrium is reached after 40-350 hours, or even more particularly after 40-100 hours after dissolution of the amyloidogenic polypeptide. For wild type IAPP under the conditions described herein, for example, the amyloid formation reaction for human IAPP reaches saturation (the end point at which amyloid fibrils are at equilibrium with soluble protein) by about 40 hrs of incubation.

In a particular embodiment of the present methods, the candidate agent is added before the time point of toxic oligomer formation. In a more particular embodiment, the candidate agent is added at the onset of the assay or before the midpoint of the lag phase of amyloid formation. In a particular embodiment with respect to IAPP, the candidate agent is added before the midpoint of the lag phase of amyloid formation. In further embodiments, the amyloidogenic polypeptide may be labeled with a detectable label or the candidate agent may be labeled with a detectable label. More particularly, the amyloidogenic polypeptide may be labeled with a first detectable label and the candidate agent may be labeled with a second detectable label. In an even more particular embodiment, the amyloidogenic polypeptide is labeled with a first detectable label and the candidate agent is labeled with a second detectable label and detectable signal of the first and/or second detectable label is altered when the first and second labels are in close proximity. As described herein, detectable labels of utility in the present methods include, but are not limited to, radioisotopes, bioluminescent compounds, chemiluminescent compounds, fluorescent compounds, metal chelates, or enzymes.

Conditions that permit self-assembly are all those conditions that do not inhibit aggregation by the peptide, polypeptide or protein. Such conditions are known in the art.

More generally, conditions that permit self-assembly involve a pH range of 1.9 to 11.0; temperature range of 1 to 100 degrees Celsius, protein concentrations ranging from nanomolar to millimolar. Ionic strength ranges from 0 to 1 molar. The solution may be buffered or unbuffered. The solution can contain organic co-solvents in the range of 0.0 to 10.0% by volume. Such solvents include hexafluoroisopropanol (HFIP), trifluoro ethanol (TFE) and DMSO. The solution may be stirred or otherwise agitated or may be quiescent.

The present methods may further comprise measuring cellular toxicity of the amyloid precursors in the presence of the candidate agent and the absence of the candidate agent, wherein a reduction in toxicity in the presence of the candidate agent indicates that the amyloid precursors are toxic intermediates and the candidate agent is an inhibitor of cellular toxicity mediated by the toxic intermediates. In accordance with results presented herein, such an embodiment could involve methods run in parallel, wherein one sample serves as negative control (e.g., no candidate agent added), one sample serves as an experimental (e.g., candidate agent added), and one sample serves as positive control (e.g., a previously identified modulator is added), and each of the samples is evaluated for cellular toxicity at the same time/s during lag phase of amyloid fibrillization for the particular polypeptide being assessed. As described herein, such samples may be co-incubated with cells from the onset of the method or may be harvested/isolated and then added to cells to determine toxicity levels and evaluate if the presence of a candidate agent alters cellular toxicity. With regard to the timing of methods wherein cellular toxicity is evaluated, such may, for example, be determined before, during, or after the midpoint of the lag phase of amyloid fibrillization. In a particular embodiment, the cellular toxicity is evaluated using pancreatic islet cells, vascular cells such as endothelial cells and smooth muscle cells, and neurons, as examples.

In yet another aspect, the inhibitor of amyloidogenic polypeptide self-aggregation into amyloids or the prophylactic and/or therapeutic agent identified using the screening methods described herein is for use in treating a subject afflicted with an amyloidoses. In an embodiment thereof, the amyloidoses is diabetes, Alzheimer's Disease (AD), or Parkinson's Disease (PD). In a particular embodiment thereof, the inhibitor of amyloidogenic polypeptide self-aggregation into amyloids or the prophylactic and/or therapeutic agent is sRAGE or a functional fragment thereof or an anti-RAGE antibody that inhibits binding of the amyloidogenic polypeptide to RAGE.

Use of the inhibitor of amyloidogenic polypeptide self-aggregation into amyloids or the prophylactic and/or therapeutic agent for the preparation of a medicament for treating a subject afflicted with an amyloidoses is also envisioned. In a particular embodiment thereof, the inhibitor of amyloidogenic polypeptide self-aggregation into amyloids or the prophylactic and/or therapeutic agent is sRAGE or a functional fragment thereof or an anti-RAGE antibody that inhibits binding of the amyloidogenic polypeptide to RAGE.

In a further aspect, a method of treating a subject afflicted with an amyloidoses is described, the method comprising administering to the subject a therapeutically effective amount of the inhibitor of amyloidogenic polypeptide self-aggregation into amyloids or the therapeutic agent identified using the screening methods described herein, wherein the administering reduces amyloidogenic polypeptide self-aggregation, thereby treating the subject afflicted with an amyloidoses. In a particular embodiment thereof, the inhibitor of amyloidogenic polypeptide self-aggregation into amyloids or the prophylactic and/or therapeutic agent is sRAGE or a functional fragment thereof or an anti-RAGE antibody that inhibits binding of the amyloidogenic polypeptide to RAGE.

In a further aspect, a method for reducing islet transplant failure in a recipient of an islet transplant is presented, the method comprising administering to the recipient of the islet transplant an agent capable of binding to human islet amyloid polypeptide (IAPP) toxic intermediates, wherein binding of the agent to human IAPP toxic intermediates inhibits human IAPP toxic intermediate binding to RAGE and thus reduces islet transplant failure due both to human IAPP-mediated toxicity and to amyloid formation and accumulation in the graft. In an embodiment thereof, the agent is sRAGE or a functional fragment thereof or an anti-RAGE antibody that inhibits binding of the amyloidogenic polypeptide to RAGE.

Also encompassed herein is a method for generating an islet transplant having resistance to islet amyloid polypeptide (IAPP) mediated cytotoxicity, the method comprising incubating pancreatic beta cells with an agent capable of inhibiting binding of human islet amyloid polypeptide (IAPP) toxic intermediates to RAGE, thereby generating an islet transplant having resistance to IAPP mediated cytotoxicity. In an embodiment thereof, the agent is sRAGE or a functional fragment thereof or an anti-RAGE antibody that inhibits binding of the IAPP toxic intermediates to RAGE.

In a further aspect, a method for generating an islet transplant having resistance to islet amyloid polypeptide (IAPP) mediated cytotoxicity is described, the method comprising introducing an expression vector that encodes an agent capable of inhibiting binding of human islet amyloid polypeptide (IAPP) toxic intermediates to RAGE, thereby generating an islet transplant having resistance to islet amyloid polypeptide (IAPP) mediated cytotoxicity. In an embodiment thereof, the agent is sRAGE or a functional fragment thereof.

In yet another aspect, the islet transplant having resistance to islet amyloid polypeptide (IAPP) mediated cytotoxicity is for use in treating a subject afflicted with diabetes. Also envisioned, is use of the islet transplant having resistance to islet amyloid polypeptide (IAPP) mediated cytotoxicity for the preparation of a medicament for treating a subject afflicted with diabetes. In an embodiment thereof, the subject is a mammal and, more particularly, is a human. In a particular embodiment, the mammal and, more particularly, the human is afflicted with type 1 or type 2 diabetes.

In a further aspect, a method of treating a subject with diabetes is presented, the method comprising administering the islet transplant having resistance to islet amyloid polypeptide (IAPP) mediated cytotoxicity to the subject. In an embodiment thereof, the subject is a mammal. In a more particular embodiment, the mammal is a human. The mammal and, more particularly, the human may be afflicted with type 1 or type 2 diabetes.

In a further aspect of the invention, a kit comprising a polypeptide capable of self-aggregation into amyloids, polypeptide self-aggregation compatible buffers, and instruction materials is provided.

### BRIEF DESCRIPTION OF THE DRAWINGS

FIG. 1. Amyloid formation by human IAPP. (A) Schematic diagram of the kinetics of amyloid formation and (B) the amino acid sequence of mature human IAPP. The sequence is shown using the standard one letter code for the amino acids. All variants have an amidated C-terminus and a disulfide bridge between Cys-2 and Cys-7.

FIG. 2. Kinetic assays reveal that h-IAPP toxic species are transiently populated intermediates. (A) AlamarBlue cell viability assays of β-cells stimulated with h-IAPP (red circles) indicate that h-IAPP intermediates are cytotoxic, while h-IAPP monomers and amyloid fibrils are not. Rat IAPP (green triangles) is not toxic at any time point. (B) Light microscopy image of viable β-cells after stimulation with monomeric h-IAPP. (C) Light microscopy image of dead β-cells after stimulation with h-IAPP intermediates. (D) Light microscopy image of viable β-cells after stimulation with h-IAPP amyloid fibrils. (E) Thioflavin-T kinetics assay of h-IAPP (red circles) shows that time points of h-IAPP induced toxicity correspond to kinetic intermediates populated in the lag phase of amyloid formation. Rat IAPP (green triangles) does not form amyloid at any time point. (F) TEM image of toxic h-IAPP intermediates shows a pre-amyloid morphology. (G) TEM image of IAPP amyloid fibrils populated in the saturation phase. All experiments were carried out side-by-side using the same peptide stock solutions. Values for AlamarBlue assays are relative to those of control cells treated with buffer alone. qRT-PCR data of (H) ILl-beta and (I) MCP1 mRNA expression after beta cell stimulation with wild type h-IAPP and rat IAPP after zero hrs (monomers and early intermediates), 7 hrs (mid-lag phase intermediates) and 24 hrs (amyloid fibrils) of incubation at 25 C in neat reaction buffer. Linear correlation plots show a direct relationship between the kinetics of amyloid formation (length of the lag phase) and the duration of toxicity. The amyloid formation kinetics of wild type and mutant h-IAPP and rat IAPP were monitored over a range of concentrations and temperatures by AlamarBlue cell viability assays and thioflavin-T kinetics assays side by side. The length of the lag phase of each reaction was plotted against respective duration of toxicity. The results indicate a linear correlation, suggesting that there is a direct relationship between the rate of amyloid formation (i.e., length of lag phase) and the duration of toxicity. Time-dependent toxicity and kinetics assays were carried out side-by-side using the same peptide stock solutions. Values for AlamarBlue assays are relative to those of control cells treated with buffer only. Toxicity is defined as <80% β-cell viability. All values for AlamarBlue and thioflavin-T kinetics assays represent means±SEM (n=3). Scale bars in TEM images represent 500 nm.

FIG. 3. Mutations that change the rate of IAPP aggregation have a correlated effect on the rate of onset and duration of toxicity. (A) AlamarBlue cell viability assay of 40 uM h-IAPP (blue circles), I26P-IAPP (green triangles), or a 1:1 mixture of the two (orange squares). (B) Thioflavin-T binding kinetics of 40 uM h-IAPP (blue circles), I26P-IAPP (green triangles), or a 1:1 mixture of the two (orange squares). (C) AlamarBlue cell viability assay of 20 uM h-IAPP (blue circles), 20 uM S20G-IAPP (purple circles), 20 uM S20K-IAPP (orange squares) and 20 uM rat IAPP (green triangle). (D) Thioflavin-T binding kinetics of 20 uM h-IAPP (blue circles), 20 uM S20G-IAPP (purple circles), 20 uM S20K-IAPP (orange squares) and 20 uM rat IAPP (green triangle). (E-H) TEM data collected at the end point of the IAPP amyloid formation reaction (E) 20 uM h-IAPP, (F) 20 uM S20G-IAPP, (G) 20 uM S20K-IAPP, (H) 20 uM rat IAPP. (I) Linear correlation plot showing a direct relationship between the length of the lag phase and duration of toxicity. The data indicate that slowing down the rate of IAPP aggregation increases the duration of toxicity. Values for AlamarBlue assays are relative to those of control cells treated with buffer only. All values for AlamarBlue and thioflavin-T kinetics assays represent means±SEM (n=3). Scale bars in TEM images represent 500 nm. h-IAPP reactions were carried out at pH 7.4 25° C.

FIG. 4. h-IAPP toxic species are transient, pre-fibrillar intermediates that lack detectable beta sheet structure. (A) Far UVCD spectra of h-IAPP intermediates populated at time point of toxicity showing the development of some partial helical structure, but no beta sheet structure. (B) 2D-IR data of h-IAPP intermediates populated at time point of toxicity shows no significant beta sheet development and supports the CD data. (C) ANS binding studies show that h-IAPP intermediates are not molten globules.

FIG. 5. h-IAPP toxic intermediates are ligands of RAGE. (A) SPR data showing sRAGE binds to 20 μM h-IAPP intermediates (green) but not h-IAPP monomers (red) or amyloid fibrils (blue). (B-D) TEM images show: (B) the absence of amyloid at zero hrs of h-IAPP incubation, (C) the absence of amyloid at 5 hrs of h-IAPP incubation and (D) the presence of amyloid after 24 hrs of h-IAPP incubation. (E) Trp fluorescence quenching experiments. The quenching of sRAGE Trp fluorescence indicates binding to sRAGE. The results show that addition of sRAGE to h-IAPP at a 1:1 molar ratio (red circles) at various times during the amyloid formation reaction leads to a wave of fluorescence quenching that mirrors the wave of toxicity. No binding is observed when sRAGE is added to rat IAPP (blue diamonds) at a 1:1 molar ratio. Results indicate that h-IAPP toxic intermediates and RAGE-binding intermediates are the same. Control proteins include 20 uM h-IAPP (purple circles), 20 uM rat IAPP (green triangles) and 20 uM sRAGE (black squares).

FIG. 6. sRAGE is an inhibitor of h-IAPP toxicity and amyloid formation. qRT-PCR studies indicate that sRAGE protects beta cells from h-IAPP induced up-regulation in (A) IL-1β and (B) MCP-1 mRNA expression. Control conditions include 20 uM h-IAPP, 20 uM Rat IAPP, 20 uM sRAGE and buffer alone. Peptide reactions assessed in (A) and (B) were incubated for 5 hrs at 25° C. before being added to cells. (C) Thioflavin-T kinetics of 20 uM h-IAPP amyloid formation carried out at 15° C. show that sRAGE is an inhibitor of h-IAPP amyloid formation. The reaction temperature was decreased to increase the duration of toxicity. sRAGE was added to h-IAPP at 1:2 molar ratio at 3.5 hrs (green), 7 hrs (purple) and 10 hrs (orange) of h-IAPP incubation. The results indicate that addition of sRAGE before the midpoint of the h-IAPP kinetic lag phase (i.e., time point of toxicity) inhibits h-IAPP amyloid formation. (F-K) TEM images demonstrate that sRAGE is an inhibitor of h-IAPP amyloid formation. sRAGE was added to h-IAPP at a 1:1 molar ratio at various time points along the h-IAPP amyloid formation reaction and TEM was recorded and compared to controls: (D) M sRAGE by itself, (E) 20 μM h-IAPP by itself after 25 hrs. sRAGE was added to h-IAPP after (F) 1.5 hrs, (G) 6.5 hrs, (H) 9.5 hrs and (I) 25 hrs of h-IAPP incubation at 25 C. (K) Difference CD data showing that sRAGE is an inhibitor of (3-sheet formation by h-IAPP. sRAGE was added to h-IAPP at a 1:1 molar ratio at various time points along the h-IAPP amyloid formation reaction: 1.5 hrs (orange), 6.5 hrs (purple), 9.5 hrs (green) and 24 hrs (black) of incubation at 25° C. Results indicate that addition of sRAGE to h-IAPP before time points of toxic intermediate formation prevents h-IAPP amyloid formation and toxicity. Addition of sRAGE at later time points of toxicity leads to significant reductions in amyloid formation. No effect is observed when sRAGE is added to amyloid fibrils. The relative fold change of controls in qRT-PCR experiments is approximately 1.0. Scale bars in TEM images represent 500 nm.

FIG. 7. Genetic deletion of RAGE or blocking RAGE-IAPP interactions protects beta cells in part from IAPP toxicity. (A) RAGE-blocking experiment. Rat INS-1 beta cells were pre-treated with increasing concentrations (0 to 150 ng/mL) of either anti-RAGE or anti-IgG antibodies, and then stimulated with h-IAPP intermediates produced after 5 hrs of incubation at 25° C. in neat reaction buffer. (B) Schematic diagram showing a RAGE-mediated mechanism of IAPP toxicity and the two potentially therapeutic effects of sRAGE: 1) prevention of IAPP toxicity and 2) inhibition of amyloid formation.

FIG. 8 shows that RAGE knock out protects aortic smooth muscle cells from IAPP toxicity.

FIG. 9. A change in the aggregation kinetics of h-IAPP leads to a change in the time course of toxicity. (A) Thioflavin-T kinetics assay of 15 uM h-IAPP (green squares), 20 uM h-IAPP (red circles) and 40 uM h-IAPP (blue diamonds). (B) AlamarBlue cell viability assays of β-cells stimulated with 15 uM h-IAPP (green squares), 20 uM h-IAPP (red circles) and 40 uM h-IAPP (blue diamonds). The results show that a decrease in IAPP concentration leads to an increase in the length of the lag phase and an increase in the duration of toxicity. Likewise, an increase in IAPP concentration leads to a shortening of the lag phase and a decrease in the duration of toxicity. Rat IAPP (black triangles) does not form amyloid at any time point. Rat IAPP (black triangles) is not toxic at any time point. (C) Linear correlation plot showing a direct relationship between the length of the lag phase and duration of toxicity. (D) Linear correlation plot showing a direct concentration-dependent relationship between the rate of aggregation and the degree of toxicity. Time-dependent toxicity and kinetics assays were carried out side-by-side using the same peptide stock solutions. Values for AlamarBlue assays are relative to those of control cells treated with buffer only. All values for AlamarBlue and thioflavin-T kinetics assays represent means±SEM (n=3). Scale bars in TEM images represent 500 nm. h-IAPP reactions were carried out at pH 7.4 25 C.

FIG. 10. sRAGE is an inhibitor of h-IAPP cytotoxicity. (A) AlamarBlue cell viability assays show that h-IAPP intermediates are toxic to mouse pancreatic islets and addition of sRAGE to h-IAPP at a 1:1 molar ratio before time points of toxic species formation prevents h-IAPP toxicity. (B) Light microscopy of hand purified mouse pancreatic islets with intact mantels after isolation from FVB mice. (C) Immunohistochemistry of pancreas sections taken from mice that were the same age, strain and metabolic condition as those used for islet isolation. Sections of paraffin embedded pancreatic tissue were stained for insulin (red), F4/80 (green) and Dapi (blue). The results indicate that pancreatic tissue was non-inflamed and insulin-positive at time of islet harvest. (D) AlamarBlue cell viability assays show that addition of sRAGE before time points of toxic intermediate formation prevents h-IAPP-induced toxicity to aortic smooth muscle cells. (E) qRT-PCR studies indicate that h-IAPP intermediates up-regulate MCP-1 and IL-1β mRNA expression in smooth muscle cells, but rat IAPP does not. Time-dependent toxicity and kinetics assays were carried out side-by-side using the same peptide stock solutions. Values for AlamarBlue assays are relative to those of control cells treated with buffer only. All values for AlamarBlue and thioflavin-T kinetics assays represent means±SEM (n=3). h-IAPP reactions were carried out at pH 7.4 25 C. The relative fold change of controls in qRT-PCR experiments is approximately 1.0.

FIG. 11 shows that rapid amyloid formation is associated with human islet graft failure. Human islets were grafted in streptozotocin-diabetic NOD/SCID recipients (n=43) as described in Potter et al (2010) Proc Natl Acad Sci 107:4305, the entire contents of which is incorporated herein by reference. Small amounts of amyloid (arrow) were detected by thioflavin S stain (blue) in grafts in normoglycemic recipients at 4 weeks posttransplant (A) but were more marked at 8 weeks posttransplant and in hyperglycemic recipients (B). Amyloid appeared adjacent to insulin-positive cells (green) and areas of apparent islet cell loss, but glucagon-positive cells (red). (Scale bar, 50 μm.) Beta cell area (C) tended to be reduced and amyloid area was increased (D) in recipients of grafts with blood glucose values >15 mM at the time of graft harvest. The number of recipients in the normoglycemic and hyperglycemic recipients were 31 and 12, respectively. *, denotes statistically significant difference from normoglycemic (<15 mM) group (P<0.05).

FIG. 12 depicts a graph showing that sRAGE inhibits the kinetics of human IAPP amyloid formation. sRAGE was added to h-IAPP at 1:2 molar ratio at 3.5 hrs (green), 7 hrs (purple), 10 hrs (orange), 52 hrs (dark blue) and 118 hrs (light blue) into the h-IAPP amyloid formation reaction. The results indicate that addition of sRAGE before the midpoint of the h-IAPP kinetic lag phase (i.e. time point of toxicity) inhibits h-IAPP amyloid formation, while addition of sRAGE after the mid-lag phase does not prevent amyloid formation.

FIG. 13 shows amino acid sequences of human and mouse alpha-synuclein.

FIG. 14 presents the nucleic (cDNA) and amino acid sequence of human RAGE as presented in Neeper et al. (J Biol Chem 267:14998-15004, 1992).

FIG. 15 presents nucleic acid sequences encoding human RAGE. Sequence A corresponds to full length human RAGE as presented in U.S. Pat. No. 7,845,697; sequence B corresponds to full length human RAGE as presented in U.S. Pat. No. 5,864,018; and sequence C corresponds to soluble RAGE (sRAGE) as presented in U.S. Pat. No. 7,845,697.

### DETAILED DESCRIPTION OF THE INVENTION

Before the present discovery and methods of use thereof are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only by the appended claims.

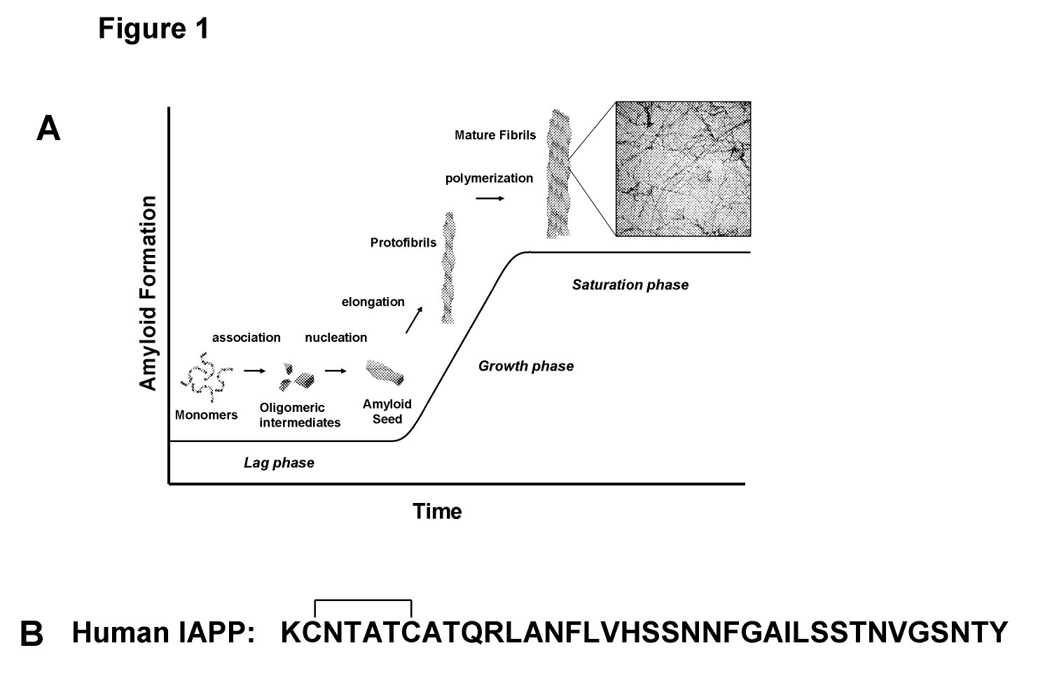

A wide range of human diseases involve the pathological aggregation of polypeptides and proteins [Chiti et al. (2006) Annu Rev Biochem 75, 333-366; Sipe et al. (1994) Crit. Rev Clin Lab Sci 31, 325-354; Selkoe. (2004) Nature Cell Biol 6, 1054-1061; Jahn et al. (2008) Arch Biochem Biophys 469, 100-117]. Protein misfolding diseases caused by the transformation of normally soluble proteins or polypeptides into ordered insoluble fibrils or amyloid plaques are commonly referred to as “amyloidoses”. The kinetics of amyloid formation typically exhibits a sigmoidal polymerization profile consisting of three observable phases: the lag phase, the growth phase and the saturation phase (See FIG. 1A). Little or no amyloid is formed in the lag phase and relatively little is known about the nature of the lag phase oligomers.

The nature of the toxic species produced during amyloid formation is not well understood. An increasing body of evidence suggests that they are pre-amyloid intermediates, although there is evidence which also support a role for amyloid fibrils in toxicity. In addition, both on- and off-pathway intermediates have been proposed to be responsible for toxicity.

The present study focuses on amyloid formation by islet amyloid polypeptide (IAPP or amylin), the causative agent of islet amyloidosis in type 2 diabetes (T2D) (FIG. 1B). In the nonpathological state, IAPP functions as an endocrine hormone involved in the regulation of satiety, carbohydrate metabolism, slowing of gastric emptying, and prevention of glucagon secretion during hyperglycemia. Pancreatic islet amyloid formation in T2D is toxic to beta cells and contributes to the decline of insulin production and secretion [Kahn et al. (1999) Diabetes, 48, 241-246; Hull et al. (2004) J. Clin. Endocrin. Metab. 89, 3629-3643]. Islet amyloid also has important implications for islet transplantation. Rapid amyloid formation in transplanted islets leads to apoptosis and transplant failure, while prevention of islet amyloid has been shown to significantly increase islet transplant survival in vivo [Selkoe. (2004) Nature Cell Biol 6, 1054-1061; Potter et al. (2010) PNAS 107, 4305]. Despite their significance, the mechanisms of IAPP amyloid formation and toxicity are not, however, understood.

We demonstrate herein that transiently populated pre-fibrillar intermediates that form during human IAPP (h-IAPP) amyloid formation are toxic to insulin producing beta cells, pancreatic islets and aortic smooth muscle cells; and up-regulate MCP-1 and IL-1β mRNA. The toxic species are loosely packed, soluble oligomers which lack significant beta sheet structure. We show that IAPP toxic species are ligands of the receptor for advanced glycation endproducts (RAGE), but non-toxic h-IAPP monomers and amyloid fibrils do not bind RAGE. Likewise, non-toxic and non-amyloidogenic rat IAPP and soluble non-toxic analogs of h-IAPP do not bind RAGE. RAGE is expressed at low levels in a wide range of differentiated mammalian cells and becomes up-regulated in amyloid-rich environments and pathological inflammatory states, including neuro-degeneration and diabetes [Hudson et al. (2008) FASEB J 22, 1572-1580; Yan et al. (2009) Journal of Molecular Medicine 87, 235-247; Yan et al. (2008) Nat Clin Pract EndocrinolMetab 4(5), 285-293; Clynes et al. (2007) Current Molecular Medicine 7, 743-751; Herold et al. (2007) J Leukoc Biol 82, 204-212]. Activation of RAGE is associated with sustained cellular oxidative stress; triggering pathological signaling cascades involved in pro-inflammatory biomarker production, apoptosis and other effector mechanisms [Yan et al. (1996) Nature 382, 685-691]. This receptor, which is a member of the immunoglobulin superfamily, has within its extracellular domain one V-type domain, two C-type domains, a transmembrane domain, and a cytoplasmic domain [Xie et al. (2008) J Biol Chem 283, 27255-27269; Park et al. (2010) JBC 285(52), 40762; Koch et al. (2010) Structure 13; 18(10), 1342].

RAGE was originally named for its ability to bind AGEs, but is now known to have several classes of ligands [Bierhaus et al. (2005) Journal of Molecular Medicine 83, 876-886; Schmidt et al. (1992) J Biol Chem 267, 14987-14997; Schmidt et al. (2001). Journal of Clinical Investigation 108, 949-955; Liliensiek et al. (2004) J Clin Invest 113, 1641-1650; Yan et al. (2000) Nat. Med. 6, 643-651]. Amyloid forming peptides and proteins constitute one class of RAGE ligand. RAGE engages pre-amyloid species of amyloid-β (Aβ) 1-40 and Aβ 1-42, serum amyloid A and prion-derived peptide [Hori et al. (1995) J Biol Chem 270, 25752-25761]. Previous studies indicate that neurotoxic Aβ peptides bind to RAGE in the brain of individuals with Alzheimer's disease (AD) and RAGE activation is postulated to play an important role in neurological dysfunction and cell death [Sturchler t al. (2008) J Neurosci. 28(20), 5149-58]. Less is known about the mechanisms of amyloid formation and toxicity by human IAPP.

As shown herein, the soluble extracellular domain of RAGE (sRAGE) is an effective inhibitor of h-IAPP amyloid formation and cytotoxicity; and blocking RAGE with anti-RAGE antibodies protects beta cells, pancreatic islets and smooth muscle cells from h-IAPP induced cytokine production and cell death. Our findings are consistent with a RAGE-mediated mechanism of h-IAPP cytotoxicity, and suggest a role for RAGE in islet amyloidosis in T2D.

Prior to the present discovery, however, the exact mechanism of human IAPP (h-IAPP) toxicity was not known. Indeed, many hypotheses have been proposed relating to potential mechanisms of h-IAPP cytotoxicity, including h-IAPP aggregate-mediated cellular membrane disruption [Janson et al. (1999) Diabetes 48, 491-498; Jayasinghe et al. (2007) BBA 1768, 2002-2009; Brender et al. (2008) J Am Chem Soc 130, 6424-6429; Engel. (2009) Chem Phys Lipids 160, 1-10]. In this view, h-IAPP disrupts membranes by forming membrane channels or inducing bilayer disorder, though it is important to note that these investigations are controversial as they make use of non-physiological lipid bilayers composed of negatively charged phospholipids that have natural affinities to bind to positively charged molecules, such as h-IAPP. Other proposed mechanisms include receptor-binding and activation of apoptosis pathways leading to cell death [Haataja et al. (2008) Endocr Rev 29 303-316]. Other data suggest that h-IAPP binding to FAS (death receptor) transduces pathological signals in cellular systems [Zhang et al. (2008) Diabetes 57, 348-356]. Studies that support a role for signal transduction-mediated mechanisms of cytotoxicity, moreover, propose that activation of cellular stress responses play an important role.

The significance of the present discovery is underscored by the aforementioned significant level of uncertainty in the field surrounding IAPP-mediated toxicity of beta cells. In that islet amyloid is the causative agent of islet amyloidosis in T2D and has important implications for islet transplantation, understanding the mechanism of cytotoxicity elucidates parameters that have significant impact on the choice of preventative and/or therapeutic intervention with respect to the selection of preventative and therapeutic agents, as well as timing of administration and delivery mode thereof.

With respect to transplantation of pancreatic islets, for example, it is known that cultured or transplanted human islets develop amyloid deposits. Accordingly, although transplantation of pancreatic islets is envisioned as a therapy option for restoring glycemic control in both T1D and T2D, the appearance of amyloid deposits has raised major concerns about the long term success of islet transplantation as a therapeutic approach [Selkoe. (2004) Nature Cell Biol 6, 1054-1061; Potter et al. (2010) PNAS 107, 4305]. In light of the present findings, it is therefore envisioned that incubating cultured human islets in inhibitors of IAPP-RAGE interactions prior to transplantation would increase the longevity of transplanted islet cells in recipients thereof. The longevity of the transplanted islet cells in recipients would be conferred by increased resistance to IAPP-mediated cytotoxicity that results from incubation with the inhibitors. Such inhibitors include sRAGE and anti-RAGE antibodies, such as those described herein, that compete with RAGE expressed on the surface of the islet cells for binding to the IAPP toxic pre-fibrillar intermediates. The introduction of expression vectors via gene transfer into islet cells in advance of transplantation to generate modified pancreatic islet cells is also envisioned and encompassed herein. Based on the present findings, useful expression vectors would comprise, for example, nucleic acid sequences that encode sRAGE, such that transplanted islet cells would express sRAGE which would act as a binding sink for IAPP toxic pre-fibrillar intermediates; or, for example, shRNA or siRNA specific for RAGE, such that transplanted islet cells would express reduced levels of RAGE.

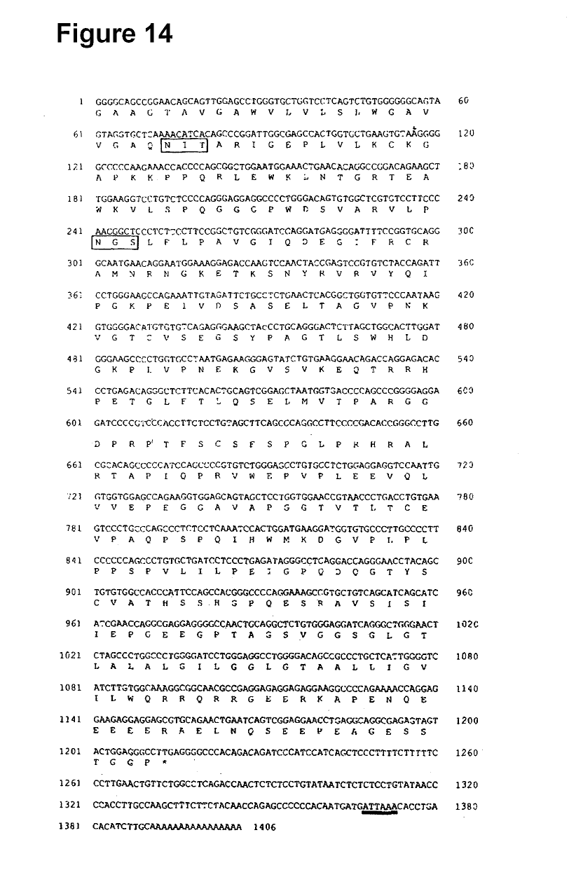

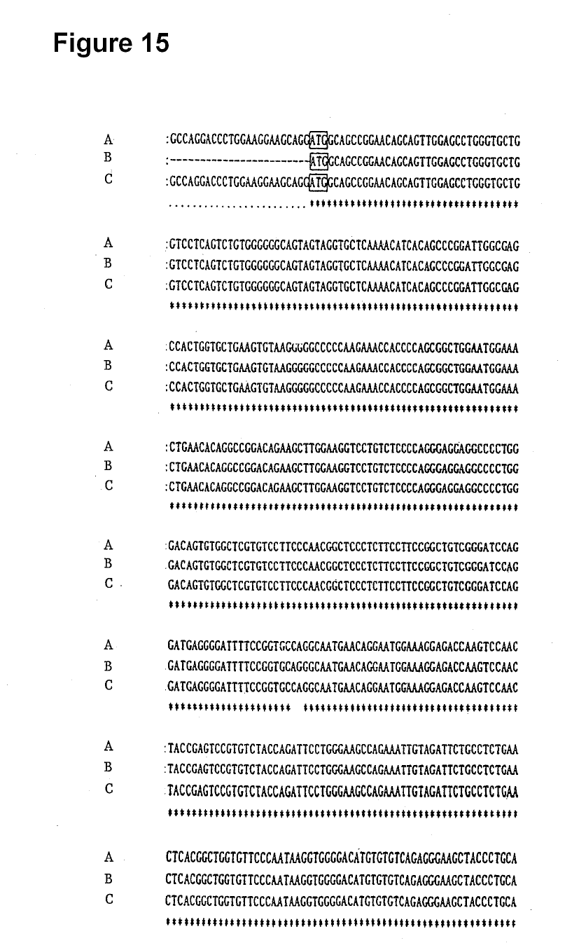

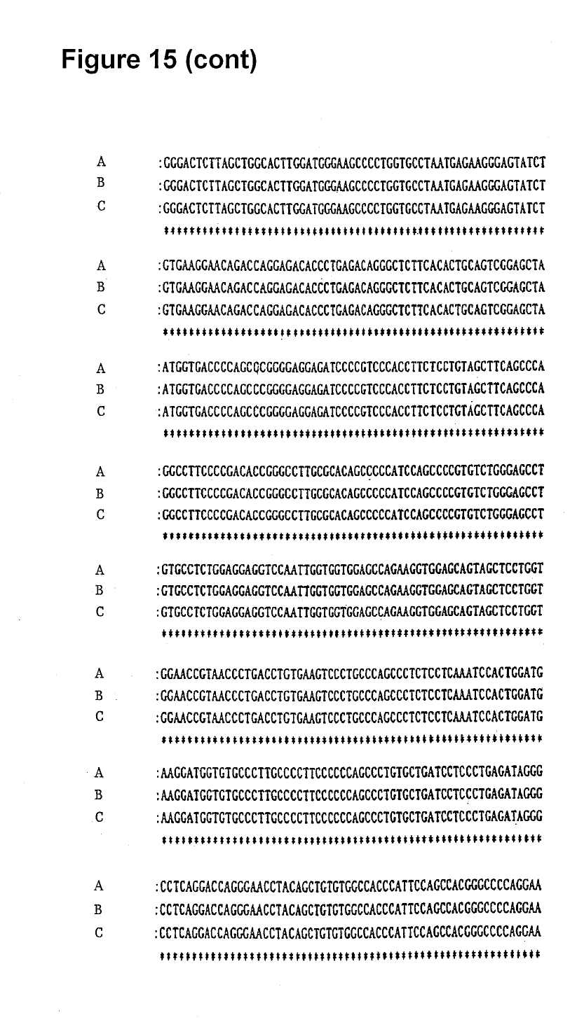

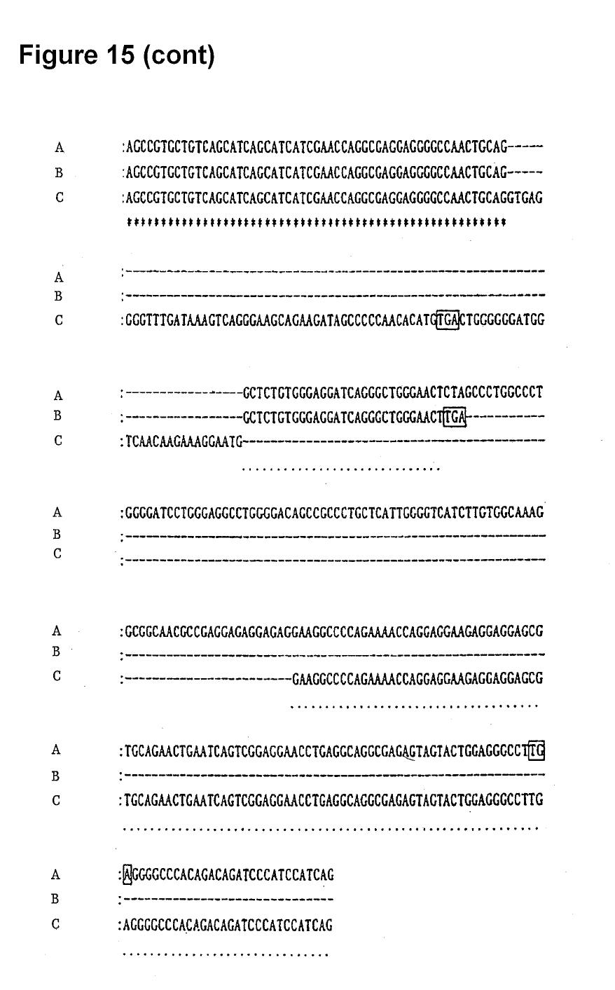

FIG. 14 presents a nucleic acid sequence of human RAGE cDNA, an exemplary nucleic acid sequence encoding human RAGE. Additional information relating to the cloning and expression of human RAGE is known in the art and detailed in, for example, Neeper et al. (J Biol Chem 267:14998-15004, 1992), the entire content of which is incorporated herein by reference. U.S. Pat. Nos. 5,864,018; 6,790,443; 7,081,241; and U.S. Pat. No. 7,485,697 provide additional information relating to the nucleic and amino acid sequences of RAGE, sRAGE, and enRAGE, the entire content of each of which is incorporated herein by reference.

Further to the above, an exemplary RAGE shRNA that can be used to inhibit endogenous RAGE expression is 5′-GCT AGA ATG GAA ACT GAA CA-3′.

The following is the amino acid sequence of full length RAGE (SEQ ID NO: 17):

AQNITARIGEPLVLKCKGAPKKPPQRLEWKLNTGRTEAWKVLSPQGGG
PWDSVARVLPNGSLFLPAVGIQDEGIFRCQAMNRNGKETKSNYRVRVY
QIPGKPEIVDSASELTAGVPNKVGTCVSEGSYPAGTLSWHLDGKPLVP
NEKGVSVKEQTRRHPETGLFTLQSELMVTPARGGDPRPTFSCSFSPGL
PRHRALRTAPIQPRVWEPVPLEEVQLVVEPEGGAVAPGGTVTLTCEVP
AQPSPQIHWMKDGVPLPLPPSPVLILPEIGPQDQGTYSCVATHSSHGP
QESRAVSISIIEPGEEGPTAGSVGGSGLGTLALALGILGGLGTAALLI
GVILWQRRQRRGEERKAPENQEEEEERAELNQSEEPEAGESSTGGP

The following is the amino acid sequence of soluble RAGE (SEQ ID NO: 18):

AQNITARIGEPLVLKCKGAPKKPPQRLEWKLNTGRTEAWKVLSPQGGG
PWDSVARVLPNGSLFLPAVGIQDEGIFRCQAMNRNGKETKSNYRVRVY
QIPGKPEIVDSASELTAGVPNKVGTCVSEGSYPAGTLSWHLDGKPLVP
NEKGVSVKEQTRRHPETGLFTLQSELMVTPARGGDPRPTFSCSFSPGL
PRHRALRTAPIQPRVWEPVPLEEVQLVVEPEGGAVAPGGTVTLTCEVP
AQPSPQIHWMKDGVPLPLPPSPVLILPEIGPQDQGTYSCVATHSSHGP
QESRAVSISIIEPGEEGPTAGSVGGSGLGTLA

Accordingly, methods for treating a patient with T1D or T2D comprising administering modified pancreatic islet cells to the patient to restore pancreatic islet cells in the patient are encompassed herein. Further to the above, use of the modified pancreatic islet cells for treating a patient with T1D or T2D and use of same in the preparation of a medicament for treating a patient with T1D or T2D so as to restore pancreatic islet cells in such a patient is also envisioned.

In accordance with the findings presented herein, assays and methods for screening to identify modulators of amyloid formation are also envisioned and described. Such modulators may function to modulate (e.g., inhibit or promote) the self-assembly of peptides, polypeptides and proteins into ordered aggregates possessing the protein quaternary cross-β structure characteristic of amyloids. In a particular aspect, the assays and methods for screening relate to the identification of modulators of islet amyloid formation. As described herein, the present inventors have discovered that IAPP is a model polypeptide for in depth stepwise analysis of the process of amyloid formation and identification of toxic intermediates generated in the pathway of amyloid formation. The present inventors have, furthermore, developed assays and methods utilizing IAPP wherein such methods may be used to screen for and to identify modulators of islet amyloid formation. In a particular aspect, such modulators are inhibitors of toxic intermediates and/or aggregation of islet amyloid and thus may be used as therapeutic agents for the treatment of diabetes. Modulators identified using the assays and methods utilizing IAPP as described herein may also have utility as agents for therapeutic intervention in amyloidosis diseases in general, including Alzheimer's Disease (AD) and Parkinson's Disease (PD).

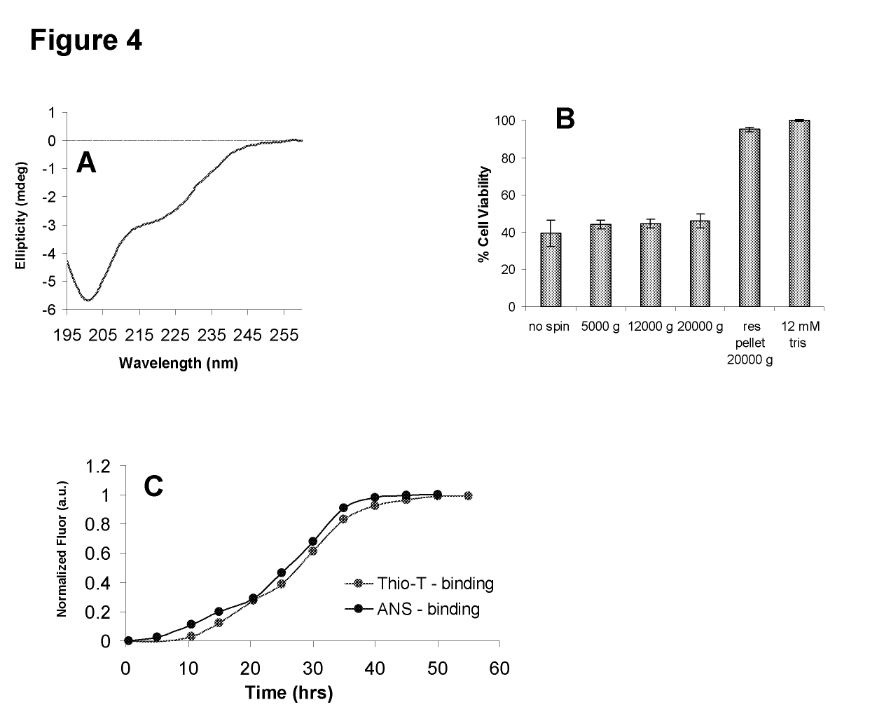

The discoveries presented herein pertaining to IAPP and toxic intermediates generated therefrom during the course of fibrillization are well applied to other amyloid forming polypeptides, such as Aβ and α-synuclein. Accordingly, definitive identification of toxic intermediates of IAPP having characteristic physical properties, which include, but are not limited to, soluble, pre-fibrillar, partially structured, but not a molten globule, and lack of detectable beta sheet character (FIG. 4) offers insight into the biology of other amyloid forming polypeptides. Given the similar polypeptide sequences and aggregation kinetics of human IAPP and other amyloid forming polypeptides (such as, for example, Aβ), toxic intermediates may also be formed during the course of fibrillization of other amyloid forming polypeptides and thus, may serve as novel targets for the development of modulatory agents that inhibit or promote amyloidogenic polypeptide aggregation.

Further to the above, modulators identified in a screening assay using a particular amyloid forming polypeptide as the indicator polypeptide may also act as modulators that alter aggregation of other amyloid forming polypeptides. Indeed, modulators identified using methods described herein may, for example, reduce or inhibit cellular toxicity of toxic intermediates of a plurality of amyloid forming polypeptides and/or may inhibit aggregation thereof. Alternatively, modulators identified using methods described herein may, for example, reduce or inhibit cellular toxicity of toxic intermediates of a plurality of amyloid forming polypeptides by promoting aggregation of same and thus, reducing the time frame in which toxic intermediates are available to interact with cells and elicit a biological effect.

### Amyloid Formation: Detection and Characterization Thereof.

Amyloid fibrils form by the aggregation of normally soluble peptides, polypeptides and proteins. It is noteworthy, however, that considerable variation is observed in the primary sequences of peptides, polypeptides and proteins. Amyloid fibrils are characterized by highly stable crossed-beta sheet organization. Techniques for detecting amyloid include: transmission electron microscopy (TEM), scanning transmission electron microscopy (STEM), and atomic force microscopy (AFM). Such techniques can be used to detect fibrils that are 5-10 nm wide and unbranched.

Methods for characterizing amyloids include, without limitation, Far UV circular dichroism spectroscopy (UVCD); Fourier transform infrared spectroscopy (FTIR); and Fluorescence-based methods, which include intrinsic fluorophores or added dyes, such as thioflavin-T or thioflavin-S, which increase in fluorescence when bound to amyloid fibrils. These and other methods are known to those skilled in the art and well with such practitioners' technical abilities.

The kinetics of amyloid formation is complex and consists of three observable phases: lag phase, growth phase, and saturation phase, which are addressed in greater detail herein below. See FIG. 1A. Recent studies suggest that amyloid precursors are the toxic species, but the identity of the toxic species during amyloid formation remains controversial. Despite intensive study, the exact mechanism of amyloid formation is unknown. Significantly, the mechanism(s) of toxicity by amyloidogenic peptides, polypeptides and proteins is unknown.

An increasing number of studies on a variety of amyloid forming peptides, polypeptides and proteins suggest that there are underlying commonalties in the mechanism of amyloid formation among different amyloid forming peptides, polypeptides and proteins, however the exact mechanism of amyloid formation has not been fully determined. There is a rich experimental and theoretical literature on protein assembly and aggregation, and various kinetic models have been used to rationalize the time course of amyloid formation. See Ferrone. Method. Enzymol. 2006; 412: 285-299; Wetzel Acc. Chem. Res. 2006; 39 (9): 671-679; Harper et al. Annual Rev. Biochem. 1997; 66: 385-407; and Oosawa et al. 1975. Academic Press, New York, N.Y., the entire contents of which are incorporated herein by reference. Extensive experimental evidence indicates that amyloid formation generally proceeds by a variation of the so-called nucleation-dependent polymerization pathway. See Ferrone. Method. Enzymol. 2006; 412: 285-299; Wetzel Acc. Chem. Res. 2006; 39 (9): 671-679. The kinetics of amyloid formation typically exhibits a sigmoidal polymerization (or fibrillization) profile consisting of three observable phases: the lag phase, the growth phase (or elongation phase) and the saturation phase (FIG. 1A). In the lag phase, oligomeric nuclei are formed in a slow process that involves unfavorable intermolecular interactions of peptide, polypeptide or protein monomers, wherein little or no amyloid is formed. Very little is known about the nature of the lag phase oligomers. These species may be formed on the pathway to amyloid formation, and lead directly to the final amyloid state (referred to as ‘on-pathway oligomers’); or they may be species that are generated during the process of self-aggregation, but are not directly on the pathway to amyloid formation (referred to as ‘off-pathway’ oligomers). The present inventors have found that the toxic oligomers of human IAPP formed during the kinetic lag phase, could be, but are not limited to, oligomers with two or more IAPP monomers per oligomer. The toxic oligomers are soluble and cannot be pelleted by centrifugation at 25,000 G for 25 minutes. The inventors have shown that these toxic oligomers are not molten globules and lack detectable beta sheet character (FIG. 4). Once a critical assembly of oligomers form an active seed, a second, more rapid growth phase (or elongation phase) proceeds exponentially during which mature fibrils polymerize. In the saturation phase, fibrils are at equilibrium with the soluble protein. The rate of amyloid formation (i.e. the length of the lag) and polymerization (length of growth phase) can vary from seconds to hours and even days depending on the experimental conditions. The lag phase can often even be abolished by seeding a solution of unaggregated peptide with a small amount of pre-formed fibrils. See Harper et al. Annual Rev. Biochem. 1997; 66: 385-407. Other factors that affect the rate of amyloid formation include protein concentration, temperature, pH, pressure, ionic strength, agitation/stirring and the presence or absence of inhibitors or catalysts (i.e. solvents, proteins and/or small molecules) that alter the rate of the nucleation and/or polymerization reactions.

Self aggregation by a peptide, polypeptide or protein can, but does not always lead to amyloid formation, therefore conditions that permit protein aggregation may include but may not be limited to conditions that promote self-assembly into classic amyloid morphology. Conditions that promote amyloid formation are those conditions that accelerate seed formation and/or accelerate polymerization of amyloid fibrils. Conditions that accelerate amyloid formation include, but are not limited to: increases in temperature, and changes in pH, pressure, ionic strength of solutions, the addition of pre-formed seeds and/or co-solvents, lipids, and other substances that can catalyze the reaction (such as negatively charged molecules like heparin sulfate, anionic lipids, small molecules, etc.).

Representative conditions include IAPP concentration ranges of 0.5 micromolar to 60 micromolar; a pH range of 4.0 to 8.0; a temperature range between 10 degrees and 37 degrees C.

The solutions can contain buffers, and salts may be added. Typical concentrations of added salt range from 0.0 molar to 200 millimolar. For example, an assay might contain IAPP at 20 micromolar, be conducted at pH 7.4 in 20 millimolar Tris HCl buffer at 37 degrees C. with 150 millimolar added NaCl.

More generally, conditions that permit self-assembly involve the pH range of 1.9 to 11.0; protein concentrations ranging from nanomolar to milimolar. Ionic strength ranging from 0 to 1 molar. The solution may be buffered or unbuffered. The solution can contain organic co-solvents in the range of 0.0 to 10.0% by volume. Such solvent conditions include hexafluoroisopropanol (HFIP), trifluoro ethanol (TFE) and dimethylsulfoxide (DMSO). The solution may be quiescent, stirred or otherwise agitated.

### Amyloid Formation and its Role in Disease

Amyloids can be classified using a variety of means. With respect to classification based on structure of the precursor protein, precursors that form amyloids can be folded precursors, such as, e.g., lysozyme, TTR, and β2-microglobin or natively unfolded precursors, such as, e.g., Aβ, α-synuclein, and IAPP. With regard to classification based on disease, diseases associated with amyloids include, without limitation, neurodegenerative diseases, such as, e.g., Alzheimer's Disease (AD) and Parkinson's Disease (PD); systemic amyloidosis, such as, e.g., systemic transthyretin related (TTR) amyloidosis and amyloid A (AA) amyloidosis; and local amyloidosis, such as, e.g., medullary thyroid carcinoma, Type 2 Diabetes, and atrial amyloid. Functional amyloids include Pme117 amyloid, Curli assembly and yeast prion. Numerous proteins that form amyloid in vitro are, however, not associated with disease.

In accordance with the present methods, a candidate agent can be identified and used for the treatment of a subject afflicted with an amyloid associated disease such as: Alzheimer's, Prion diseases Parkinson's, Huntington's, Type-II Diabetes, Familial British dementia, Hereditary cerebral amyloid angiopathy, Familial amyloid polyneuropathy III Senile systemic amyloidosis, Gelsolin Amyloid Disease, Primary systemic amyloidosis, Secondary systemic amyloidosis, Familial non-neuropathic amyloidosis, Dialysis-related amyloidosis, Amyotrophic lateral sclerosis (ALS), Pick's Disease, Hereditary renal amyloidosis, Pituitary-gland amyloidosis, Injection-localized amyloidosi, Atrial amyloidosis, or AL cardiac amyloidosis. Table 1 sets forth an exemplary list of relevant amyloid forming polypeptides and proteins associated with human disease. Candidate agents identified using the screening methods described herein may also be useful for preventing disease onset.

TABLE 1
Prevalent pathological and functional amyloid and amyloid-
like structures, and their major protein components.

| Disease or amyloidosis | Aggregating protein | Amyloidosis type |
|---|---|---|
| Amyloidotic polyneuropathy; | Transthyretin | Systemic |
| familial amyloid cardiopathy; | | |
| senile systemic amyloidosis | | |
| Finnish hereditary amyloidosis | Fragments of gelsolin mutants | Systemic |
| Huntington's disease | Human huntingtin with expanded | Local |
| | polyglutamine repeats | |
| Tuberculosis and Rheumatoid | Serum amyloid A | Systemic |
| arthritis | | |
| Pulmonary alveolar proteinosis | Surfactant protein C (SP-C) | Local |
| Cerebral autosomal dominant | Notch 3 | Systemic |
| arteriopathy with subcortical infarcts | | |
| and leukoencephalopathy | | |
| (CADASIL) | | |
| Cystic fibrosis, AA (secondary) | Amyloid A protein | Systemic |
| amyloidosis | | |
| Serpinopathies | Serpins | Systemic |
| Aortic medial amyloidosis | Medin (lactadherin) | Local |
| Atrial amyloidosis | Atrial natriuretic factor | Systemic |
| Intracytoplasmic neurofibrillary | Tau protein | Local |
| tangles; Tauopathies | | |
| Alzheimer's disease; inclusion- | Amyloid β peptide 40 and 42 | Local |
| body myositis; Down's syndrome; | | |
| retinal ganglion cell degeneration | | |
| in glaucoma; cerebral β-amyloid | | |
| angiopathy | | |
| Hereditary cerebral haemorrhage | Mutants of amyloid β peptide | Local |
| with amyloidosis | | |
| Familial British dementia | ABri | Local |
| Familial Danish dementia | ADan | Local |
| Type II diabetes, pancreatic islet | Amylin, also known as IAPP | Local |
| amyloidosis | | |
| Parkinson's disease and other | α-Synuclein | Local |
| synucleinopathies | | |
| Familial amyotrophic lateral sclerosis | Superoxide dismutase (SODI); | Local |
| | TDP-43 | |
| Creutzfeldt-Jakob disease; bovine | Prion protein | Local and |
| spongiform encephalopathy (mad | | Systemic |
| cow disease); Gerstmann-Sträussler's | | |
| syndrome | | |
| Injection-localized amyloidosis | Insulin | Local |
| Fibrinogen amyloidosis | Variants of fibrinogen α-chain | Local |
| Lysozyme amyloidosis | Mutants of lysozyme | Systemic |
| Restrictive amyloid heart; | Apolipoprotein AI | Local |
| ApoAI amyloidosis | | |
| ApoAII amyloidosis | Apolipoprotein AI | Local |
| ApoAIV amyloidosis | N-terminal fragment of | Local |
| | apolipopprotein AIV | |
| Pulmonary alveolar proteinosis | Lung surfactant protein C | Local |
| Glucagon amyloid-like fibrils | Glucagon | Nonpathologic |
| Cutaneous lichen amyloidosis | Keratins | Systemic |
| Medullary carcinoma of the thyroid | Calcitonin | Local |
| Cataract | y-Crystallins | Local |
| Hemodialysis-related amyloidosis | Beta 2-microglobulin (Beta2m) | Systemic |
| Cutaneous amyloidosis; localized | Lambda immunoglobulin light | Systemic |
| amyloidosis of the skin | chains of variable subgroup I | |
| Corneal amyloidosis associated | Lactoferrin | Systemic |
| with trichiasis | | |
| Icelandic hereditary cerebral amyloid | Mutant of cystatin C | Local |
| angiopathy | | |
| Pituitary prolactinoma | Prolactin | Local |
| Hereditary lattice corneal | Mainly C-terminal fragments | Systemic |
| dystrophy | of kerato-epithelin | |
| AL (light chain) amyloidosis | Monoclonal immunoglobin | Systemic |
| (primary systemic amyloidosis) | light chains | |
| AH (heavy chain) amyloidosis | Immunoglobulin heavy chains | Systemic |
| Fibrinogen amyloidosis | Fibrinogen | Local |
| Critical illness myopathy (CIM) | Hyperproteolytic state of | Local |
| | myosin ubiquitination | |
| Silks of insects and spiders | ADF-3, ADF-4, and other silk | Functional |
| | proteins | |
| Pmel17 amyloid (protection of | Pmel17 | Functional |
| melanocytes against melatonin | | |
| toxicity during pigment-melanin | | |
| biosynthesis) | | |
| Factor XII amyloid (activator of | Factor XII protein | Functional |
| hemostatic system) | | |
| Curli amyloid (cell-cell adhesion | Curli E. coli Protein (curlin) | Functional |
| molecules) | | |
| Functional prions | Yeast and fungual prions | Functional |
| | Sup35, URE2p, Rnq1P, HET-s | |

### IAPP

Notably, IAPP is one of the most amyloidogenic sequences known. In pathological states, islet amyloid accelerates late stage diabetes and causes serious complications for islet transplantation, thereby limiting the utility of such transplantation for the treatment of diabetes. Indeed, rapid amyloid formation in transplanted islets leads to apoptosis and transplant failure. Prevention of islet amyloid has been shown to significantly increase islet transplant survival in vivo. See Potter and Abedini et al. (2010) Proc Natl Acad Sci 107:4305, the entire contents of which is incorporated herein in its entirety. In a nonpathologic state, however, IAPP normally participates in a variety of functions, including: satiety, carbohydrate metabolism, slowing of gastric emptying, and prevention of glucagon secretion during hyperglycemia.

Accordingly, in a particular aspect, the present methods are directed to screening for a therapeutic agent capable of reducing or eliminating the formation of toxic oligomers of IAPP, wherein a therapeutic agent so identified is useful in the prophylaxis or treatment of a subject afflicted with T2D or T1D. In a particular embodiment, the subject afflicted with T1D has received a pancreatic islet transplant and the administration of the therapeutic agent reduces or prevents formation of IAPP toxic oligomers that impair transplant viability. IAPP toxic oligomers may also serve as diagnostic markers.

With respect to experimental procedures involving islet transplantation, islets are typically taken from the pancreas of a deceased organ donor. More particularly, the islet cells are removed from the pancreas using specialized enzymes. Transplantation occurs soon after islet removal as a consequence of the fragile nature of the isolated islet cells. In short, the islets are purified, processed, and transferred into another person (i.e., a recipient in need thereof, typically a subject with T1D).

Once implanted, the beta cells in these islets begin to synthesize and release insulin.

Typically a patient receives at least 10,000 islet “equivalents” per kilogram of body weight, extracted from one or two donor pancreases. Patients often require multiple transplants to achieve insulin independence.

Transplants are often performed by a radiologist, who uses x-rays and ultrasound to guide placement of a catheter—a small plastic tube—through the upper abdomen and into the portal vein of the liver. The islets are then infused slowly through the catheter into the liver. The patient receives a local anesthetic and a sedative. In some cases, a surgeon may perform the transplant through a small incision, using general anesthesia. In other cases, islet transplantation takes place during general surgery in which both kidney and islet transplantation procedures take place during one operation.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure and so forth.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

The phrase “flanking nucleic acid sequences” refers to those contiguous nucleic acid sequences that are 5′ and 3′ to a particular nucleic acid or nucleic acid recognition site.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, 90%, 95%, 97%, 98%, or 99% of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence. The terms “percent similarity”, “percent identity” and “percent homology” when referring to a particular sequence are used as set forth in the University of Wisconsin GCG software program and are known in the art.

The present invention also includes active portions, fragments, derivatives and functional mimetics of amyloid forming polypeptides or proteins of the invention. An “active portion” of an amyloid forming polypeptide refers to a peptide that is less than the full length polypeptide, but which retains measurable biological activity. In a particular aspect thereof, the measurable biological activity is the ability to aggregate under conditions that permit self-assembly or promote aggregation of the full length amyloid forming polypeptide.

A “fragment” or “portion” of an amyloid forming polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. A “derivative” of an amyloid forming polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of the original amyloid forming polypeptide.

Different “variants” of amyloid forming polypeptides exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the amyloid forming polypeptide, (c) variants in which one or more amino acids include a substituent group, and (d) variants in which an amyloid forming polypeptide is fused with another peptide or polypeptide such as a fusion partner, a protein tag or other chemical moiety, that may confer useful properties to an amyloid forming polypeptide, such as, for example, an epitope for an antibody, a polyhistidine sequence, a biotin moiety and the like.

To the extent such analogues, fragments, derivatives, mutants, and modifications, including alternative nucleic acid processing forms and alternative post-translational modification forms result in derivatives of an amyloid forming polypeptide that retain any of the biological properties of the amyloid forming polypeptide, they are included within the scope of this invention.

The term “functional” as used herein implies that the nucleic or amino acid sequence is functional for the recited assay or purpose.

The term “functional fragment” as used herein implies that the nucleic or amino acid sequence is a portion or subdomain of a full length polypeptide and is functional for the recited assay or purpose.

An exemplary functional fragment of sRAGE, for example, comprises or consists of the variable (V) domain, the V-C1 fused domains, the C2 domain, and the fully intact V-C1-C2 domains.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which are placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity of the isolated polypeptide, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

The compositions containing the molecules or compounds of the invention can be administered for pharmaceutical or therapeutic purposes. In pharmaceutical or therapeutic applications, compositions are administered to a patient suffering from an amyloidosis disease in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines, chemokines, and the like. Immune responses may be measured both in in vitro and in various cellular or animal systems.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

As used herein, an “amyloidogenic polypeptide” is a polypeptide capable of self-assembly to form amyloids under conditions that permit self-assembly.

As used herein, the term “lag phase” refers to the length of time preceding amyloid formation as detected by thioflavin-T fluorescence.

As used herein, the term “equilibrium” refers to the steady state balance between the conversion of soluble IAPP to IAPP amyloid fibrils and the conversion of IAPP amyloid fibrils to soluble IAPP.

As used herein, the phrase “conditions that permit self-assembly of an amyloidogenic protein” refers to any condition that allows for IAPP to stably interact with itself.

As used herein, an “islet transplant having resistance to islet amyloid polypeptide (IAPP) mediated cytotoxicity” refers to an islet transplant that has been treated to minimize IAPP mediated cytotoxicity. As described herein, such a transplant may, for example, have been engineered to express sRAGE (i.e., transfected with a construct that encodes sRAGE) or incubated ex vivo in the presence of sRAGE or blocking antibodies that inhibit engagement of cell surface expressed RAGE with IAPP. Such transplants may, moreover, be transplanted in the presence of sRAGE or blocking antibodies that inhibit engagement of cell surface expressed RAGE with IAPP.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### Preparation of Amyloid Forming Polypeptide-Encoding Nucleic Acid Molecules and Amyloid Forming Polypeptides

### A. Nucleic Acid Molecules

Nucleic acid molecules encoding amyloid forming polypeptides of the invention may be prepared by two general methods: (1) Synthesis from appropriate nucleotide triphosphates; or (2) Isolation from biological sources. Both methods utilize protocols well known in the art. The availability of nucleotide sequence information, such as a full length amyloid forming polypeptide gene, such as, for example, the IAPP gene having the nucleic acid sequence of SEQ ID NO: 1, enables preparation of an isolated nucleic acid molecule of the invention by oligonucleotide synthesis. Synthetic oligonucleotides may be prepared by the phosphoramidite method employed in the Applied Biosystems 380A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Synthetic DNA molecule constructed by such means may then be cloned and amplified in an appropriate vector. Nucleic acid sequences encoding an amyloid forming polypeptide may be isolated from appropriate biological sources using methods known in the art. In a preferred embodiment, a full length amyloid forming polypeptide gene is isolated from an expression library of bacterial origin.

Nucleic acid sequences encoding the following polypeptide and peptide sequences are also encompassed herein. A nucleic acid sequence for human pro-IAPP (SEQ ID NO: 1) is as follows:

```
      1 gggtatataa gagctggatt actagttagc aaatgagggg gtaaatattc cagtggatac
    61 aagcttggac tcttttcttg aagctttctt tctatcagaa gcatttgctg atattgctga
  121 cattgaaaca ttaaaaggta aagaatttcc tatttctggg aaagttttat ttatttagag
  181 aaatgcacac ttggtgttaa attcatggtt tatttcaaag aaaggctaaa gggagaatgt
  241 attacaatat aaatgttcag attgcttaga gaaggaaatt gggaaagtaa aaatctcgaa
  301 attacttgaa aagtggacaa tattaaggga ctgtatcaat aaaaattttg atccttgtaa
  361 attacgtttt aaaaagatgt ttcttttaaa aactaagctc taatttaaaa ttacatcaat
  421 tagaactgta agaaatctct tgatttcagt gctggattat tctttgcaga aaatttgaga
  481 agcaatgggc atcctgaagc tgcaagtatt tctcattgtg ctctctgttg cattgaacca
  541 tctgaaagct acacccattg aaaggttggt aactttaaaa tcctgtttct ttgtaacttt
  601 tgtaaagtgt gagaaaatta gaattaaata ctgtcaaata actacagcct tagatttctg
  661 actatatcat acttaagaac agtaccttca gctattccat tgttccttga attctgtgtt
  721 ctttaaagaa taacaccagt ggcaaataaa tatctttgat ggaacttctg acagacagga
  781 atggataatt ccagttttgt caagaaatat actcttggaa cttagagggg caaagccaga
  841 acatgaagcg ggaaaaaaat caaaaggtag taatttcttc tatattaacc tgatactgaa
  901 acaaaccaga gaaacttaac taaagcatat atttttatac caagtggatt ctttttgtat
  961 atattactta gatttttgtt ttcctcagat gtctctggaa atgttaaaaa cttttacatc
1021 ttgtggaaat ggaaatgtat agaaataatc aggagcaaat taatgttttt aagaaatgaa
1081 atctaaaaga agtagttaaa aagccatttg ctgttggggg atttattcta tattgctagc
1141 tagctattct gtgagtgaaa cagatttata aaaagttatt cttcttatta cttctaagct
1201 gctataaact taatactttt taaaattact ttcagtaggt agcatgtatg tcaggatttc
1261 ctgggaagtc ttattacgaa aggtttcatg tcatttaaat ggtaattaag gacatctaac
1321 aactatgtca cgtaaaactc ttagagtagt taaaattttc aaactgagat tttaaaactg
1381 taatttattt aaagggttat taagttcaaa tatgtgcata agtcataaat aacatagtga
1441 ggatttgttt gtgcctaaat tagttttgct ccatatagtc ttatgggact gaacttacac
1501 actctttaac accaaggaga attaagttta cctttgtaaa gagtgtgcat gtcatattat
1561 aattcttctc attagaatga tcgtcatctt gtctttgttt tccttcgagg tagtttttct
1621 tggaagccca tagcaatatg caaagatttc tacagcacct acgtataata aatagcaaga
1681 atcattatca gaggcttttt gtcatttcaa ggcttattta gtttacaggg tgttcttctc
1741 agaactgact gtaattttct atttgctttt tcataaaaat aacttttaaa atgacatgaa
1801 gtttctgata agcagaatat ctgaatgatg acaggaaaat cagtagtatt tcctagtata
1861 tctgtttata tcttgatact ttctttcaat agatatagaa atttactaag cacttttacc
1921 ctctcttttt tttattttat tttgagacag ggtctctctc tctctctctc tctgtctctg
1981 tcacccaggc tgctggagca cagtggtaca atcatggctc actgtagcct tgacattcta
2041 ggctcaggtg atcctcccac ctcagcctcc caagtagctg ggaccacagg cacctgccac
2101 catacccagc taattgtttt atctttattt tatagggaaa gggtctccct atgatgccca
2161 ggctggtgtc aaactcctgg gctcaagcct tcctcctgcc tcagtcttcc aaaattctgg
2221 gattataaga gtgagccact gaacccagac cattatgttt ttatagatgt ttgtttatta
2281 tgagagaaac ttcacttaga aatagagcaa tatgtaatat aatattactt gttataaaat
2341 tattttgatg ttagtctcac aatctttaac tttgaattat tagaaatctt gtaaaacatt
2401 cttcaaattg ctttttaata tgttgcctga aatgagtatg tttgaacatt tgttaaaggg
2461 agtatgattt gtcatgctga gatgttaaat catgtactat tctacatatc tcacagaaag
2521 ctaggaaaat ctatggggaa aatgtgtcaa attttaaact ctttttaaaa aataaaacta
2581 acattattca atgtcatttt cctcacaaaa tttaatcatc tcatttgaga ttttttcaat
2641 ttgtaaatgt atgaaatagg ataaaaggat cacatacttt cccaccaact tttttacact
2701 cccttgtaaa tatctgcctg gcaggtaatc aaaggatagt taaaaatata attacataga
2761 tgccaagatg caatcactag gatctccctg caggagctca catacttcca cagatgaatg
2821 ttaaggctga gagcagggac tcactttaaa gtcattttga aaactctgga gagacaattt
2881 aaaagagagg caatttaaga gttatacttt ggcttattgt catctctgtt taaactctct
2941 taaagtcaag aatttccatg tgtgtatgtg cctgtaagtg gtctacagct ttaatgtttg
3001 ttactagctc gtatgttacc tgtccaggta gtcaatgaga aaaaaatgcc tgaaaccagg
3061 gaggtaatgc cttttattaa ccatttcaga caactttttc catcctaaag attgctttag
3121 atagaatctt atatatactg aatagtatat ttagatgaaa agtctttttt agaaaagcaa
3181 tttcacaaat atgataaaaa cataaatgct tttactattt cttctaagtg gaatgatggc
3241 ccatctagct aactcaaata aggtaacatt ttatttagaa caattttaaa ttatattatt
3301 gaccttccaa ccaattatca aaataccact cagcatttag catataaagt atttcacact
3361 gtgcttcagt catatgctaa acatatcttg gaacagatat tacctttgaa tcttctcaat
3421 ttgacccata attttccttt attacttttt ttgagatgtt tggaccaaat tccaattttt
3481 actgtttttc aagaaaagta agtattttag aattcaatgc aaatgtatga aattactagt
3541 tcaatcctta aagcataaat cactcttttg aaatgtacat tggtcatatt tatggtacct
3601 tcaaaaataa ataattgaac agatagtgtg aatgagattg atataggtta aataattaga
3661 tcccaaagtg gttttctttg cccagataat ttgttcaaac atttgtcagc atacacttac
3721 attcaacaag tatccagttc acctaatgct gtaagaagtt ttctgtactt aggaagaaat
3781 atgggagtaa aatttaaaaa aaaaacagtt tcacatgaga ttattaaata tttactctta
3841 ggctatctct acttagagat agagataatg aaatactccc cacacaaggt aaacacaatg
3901 agataaatcc attgcatttg agtcccagat tatgcatatc cactggctcc tggacattga
3961 gtttttagcc ctataactat ttcattttcc atttacccta agtttcacca atattttgat
4021 ttctatggag ctgaaaacta aaacatttct ctaactttcc taataatcag caaagaggaa
4081 gcaatgttat tattctgcat ccatttccga tatcgtttta aaagcacatt gaaacaaaag
4141 gctgtcaaaa aaatagagtt ggtatacaaa taaatgtctt aaataaaaac ataagttaaa
4201 attaaatgaa ttatttaatg tgtggttatg atttctgagt ttataagtat tattatgcac
4261 ttcttccagg tggctagaaa aatgtgatga atattaatac cattgacata aaaagtcttt
4321 tggttttaac atttaaccta gtcttatcat taaaattctt gaaagcataa gatccaagca
4381 ggaaaatgta tttatgctaa aagtaataaa actctcacac tgcaatagag tacctgaaca
4441 ggtgatagat ttgattcttt tggagacttt atgatattct ctttttttga catacttttt
4501 atgacattat tttttacttt attatatttc attttattgt tttaagaaca aagcatgata
4561 tctacccttt taacaaattt ttaagcatgc aatacattat tctggattat gtgcaaaatg
4621 ttgggcagca gatctctaga gcttagtcat cttgcttgac tgaagctgta cacccaatgg
4681 ttagtaactc cctatttccc cctctccctt gcccctgata accaccattc cactctttaa
4741 ctcatgaatt tgactatttt aaatacttca tatacatgga accaagtggt atttatcttt
4801 ctatgactag cttctttcac tcaacctaat gtcctcaagg ttcatccgtg tgttgcatat
4861 tgcagaattc ccttatgaca tttcttgcat aacactcctg attcaattat ctcaaggaac
4921 ttaaagacta agtaatgctg ctttattctt attggaaaga tgtagaaata attattttta
4981 aatttcttca tatttcagat tacatataaa ttttaccttc taaattcttt ttatatatta
5041 aaaataaatt cttcaagatt tttaaaaatg taagacaaag acactgttat tttgattata
5101 tgtaatatat tctgaatttc caaaggaaga cttttaactg agaaatgcaa cattgactgt
5161 aatgaaagat gttgtatgat tttcaattgt tatttcaagg tgtcaaaaaa aaatctcagc
5221 catctaggtg tttgcaaacc aaaacactga gttacttatg tgaaaattgt ttctttggtt
5281 ttcatcaata caagatattt gatgtcacat ggctggatcc agctaaaatt ctaaggctct
5341 aacttttcac atttgttcca tgttaccagt catcaggtgg aaaagcggaa atgcaacact
5401 gccacatgtg caacgcagcg cctggcaaat tttttagttc attccagcaa caactttggt
5461 gccattctct catctaccaa cgtgggatcc aatacatatg gcaagaggaa tgcagtagag
5521 gttttaaaga gagagccact gaattacttg cccctttaga ggacaatgta actctatagt
5581 tattgtttta tgttctagtg atttcctgta taatttaaca gtgccctttt catctccagt
5641 gtgaatatat ggtctgtgtg tctgatgttt gttgctagga catatacctt ctcaaaagat
5701 tgttttatat gtagtactaa ctaaggtccc ataataaaaa gatagtatct tttaaaatga
5761 aatgtttttg ctatagattt gtattttaaa acataagaac gtcattttgg gacctatatc
5821 tcagtggcac aggtttaaga acgaaggaga aaaaggtagt ttgaaccttg gtaaattgta
5881 aacagctaat aatgaagtta ttcttgacat gagaaaatca gtaattggac caggcgcggt
5941 ggctcttgcc tgtaatccca gcactttggg aggccgaggc aggcagatca caaggtcagg
6001 agttcgagac cagcctgacc aacatggtga aaccctgtct ctactaaaaa tacaaaaatt
6061 agccgggggt ggtgacatgt gcctgtaatc ccagctactc aggaggctaa ggcaggagaa
6121 tcgcttaaac ccaggaggcg gaggttgcag tgagccgaga ttgcaccact gcactccagc
6181 ctgggtggca gagtgagact cgtctcaaaa aaaagaaaga aaattagtaa ttgtaagtac
6241 ccctgataag caaattagta attgtcaata cccctgttaa gcaattcctt tttgcagtat
6301 atttctgaaa tgacagaatg ctgttttaaa aacaaagaaa taaaatcctg ctcctgactc
6361 ggtcaaaata ttttttaaag tctattgttt gttgtgcttg ctggtactaa gaggctattt
6421 aaaagtataa aactgctttg tatccatgag ggtttcattg tgtgttagca gcagtgagct
6481 tctattaaat gtatatgtca tttattttgt ttaagtggct ttcagcaaac ctcagtcata
6541 ttcttatgca gggtattgcg aaacaacttg tgttctatta atcgtgtctt caattaaaag
6601 accacagact tctggaaact ctttgctgta taagaattat ttcttttgtt taacaaatta
6661 gacatttctg gcagaggtta tgtatatgat acactttttt tgatagcagc tgcaatgttg
6721 gacagaagat gaaatgcttt gctttgagtc agattcttat gaatatctgc ttttccctga
6781 ctttgagtta ggtagctttg gaagtagcat taattcagat aaactgccat catgctgcgt
6841 tatgccattt ctaaagacac tcaacttgta cttttaaaaa aatagaaaaa ataagcattt
6901 caatctaagt ggaaatttga ctcattgact tacatttcta agttaaaatt tccctttatg
6961 aagtgtgcct taggttacca aattgtagag gctttcgttg gtggtggtaa gtggtagcgg
7021 tagtgagtgt atagaggcag ggaaatatat ttataataaa ttctatgtca tgaattacat
7081 attgaaataa ataggtgaat atacaaattt ata
```

Shown below are the proform of human IAPP, (human pro-IAPP) and partially processed forms of human pro-IAPP which form amyloid. The proteins can contain a disufide bond between the two Cys residues. The above sequences are shown using the standard 1 letter code for the amino acids. (A) Human pro-IAPP. The C-terminal residue in pro human-IAPP has a free carboxy group. (B) Partially processed Human pro-IAPP, in which the C-terminal flanking sequences have been correctly processed. (C) Partially processed Human pro-IAPP, in which the C-terminal flanking sequence has been correctly processed and the C-terminus amidated.

A)
(SEQ ID NO: 14)
TPIESHQVEKRKCNTATCATQRLANFLVHSSNNFGAILSSTNVGSNTYG
KRNAVEVLKREPLNYLPL
B)
(SEQ ID NO: 15)
TPIESHQVEKRKCNTATCATQRLANFLVHSSNNFGAILSSTNVGSNTYGKR
C)
(SEQ ID NO: 16)
TPIESHQVEKRKCNTATCATQRLANFLVHSSNNFGAILSSTNVGSNTY

Additional noteworthy IAPP sequences are listed below:

Human IAPP (SEQ ID NO: 2):
KCNTATCATQRLANFLVHSSNNFGAILSSTNVGSNTY
The S20G mutation of human IAPP (SEQ ID NO: 3):
KCNTATCATQRLANFLVHSGNNFGAILSSTNVGSNTY
Feline and Canine (SEQ ID NO: 3)
KCNTATCATQRLANFLIRSSNNLGAILSPTNVGSNTY
Pig (porcine; SEQ ID NO: 5):
KCNMATCATQHLANFLDRSRNNLGTIFSPTKVGSNTY

Sequences of mature IAPP are shown below, using the standard 1 letter code for the amino acids. All variants have an amidated c-terminus and a disulfide bridge between residue-2 and residue-7.

1-26:
(SEQ ID NO: 6)
KCNTATCATQRLANFLVHSSNNFGAI
27-37:
(SEQ ID NO: 7)
LSSTNVGSNTY
8-20:
(SEQ ID NO: 8)
ATQRLANFLVHSS
10-19:
(SEQ ID NO: 9)
TQRLANFLVHS
17-37:
(SEQ ID NO: 10)
VHSSNNFGAILSSTNVGSNTY
30-37:
(SEQ ID NO: 11)
TNVGSNTY
20-29:
(SEQ ID NO: 12)
SNNFGAILSS
8-37:
(SEQ ID NO: 13)
TQRLANFLVHSSNNFGAILSSTNVGSNTY

Fragments of IAPP may also form amyloid and be cytotoxic. Such fragments include residues 1-26; residues 27-37; residues 8-20; residues 10-19; residues 17-37; residues 30-37; and residues 20-29. Fragments may have free carboxy termini or the C-termini may be amidated. Fragments may contain a disulfide bond between residues 2 and 7 or the disulfide may be reduced. Fragments may have a free amino-terminus or the amino-terminus may be amidated. Sequences are shown using the standard 1 letter code for the amino acids.

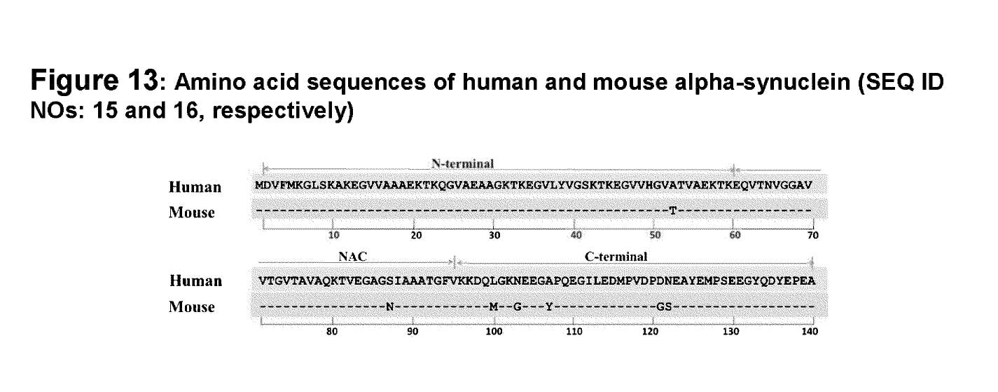

FIG. 13 depicts the amino acid sequences of human and mouse alpha-synuclein. Human alpha-synuclein is an exemplary amyloidogenic polypeptide.

In accordance with the present invention, nucleic acids having the appropriate level of sequence homology with the protein coding region of, for example, SEQ ID NO: 1 may be identified by using hybridization and washing conditions of appropriate stringency. For example, hybridizations may be performed using a hybridization solution comprising: 5×SSC, 5×Denhardt's reagent, 0.5-1.0% SDS, 100 micrograms/ml denatured, fragmented salmon sperm DNA, 0.05% sodium pyrophosphate and up to 50% formamide. Hybridization is generally performed at 37-42° C. for at least six hours. Following hybridization, filters are washed as follows: (1) 5 minutes at room temperature in 2×SSC and 0.5-1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C.16.6 Log [Na+]+0.41(%G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

As can be seen from the above, the stringency of the hybridization and wash depend primarily on the salt concentration and temperature of the solutions. In general, to maximize the rate of annealing of the two nucleic acid molecules, the hybridization is usually carried out at 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 micrograms/ml denatured salmon sperm DNA at 42° C. and wash in 2×SSC and 0.5% SDS at 55° C. for 15 minutes. A high stringency hybridization is defined as hybridization in 6×SSC, 5×Denhardt's solution, 0.5% SDS and 100 micrograms/ml denatured salmon sperm DNA at 42° C. and wash in 1×SSC and 0.5% SDS at 65° C. for 15 minutes. A very high stringency hybridization is defined as hybridization in 6×SSC, 5×Denhardt's solution, 0.5% SDS and 100 micrograms/ml denatured salmon sperm DNA at 42° C. and wash in 0.1×SSC and 0.5% SDS at 65° C. for 15 minutes.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell.

Amyloid forming polypeptide-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of SEQ ID NO: 1. Such oligonucleotides are useful as probes for detecting or isolating amyloid forming polypeptide genes.

It will be appreciated by persons skilled in the art that variants of sequences encoding amyloid forming polypeptides exist, and must be taken into account when designing and/or utilizing oligonucleotides of the invention. Accordingly, it is within the scope of the present invention to encompass such variants, with respect to the amyloid forming polypeptide sequences disclosed herein or the oligonucleotides targeted to specific locations on the respective genes or RNA transcripts. With respect to the inclusion of such variants, the term “natural allelic variants” is used herein to refer to various specific nucleotide sequences and variants thereof that would occur in a given DNA population. Genetic polymorphisms giving rise to conservative or neutral amino acid substitutions in the encoded protein are examples of such variants. Additionally, the term “substantially complementary” refers to oligonucleotide sequences that may not be perfectly matched to a target sequence, but the mismatches do not materially affect the ability of the oligonucleotide to hybridize with its target sequence under the conditions described.

Thus, the coding sequence may be that shown in SEQ ID NO: 1, or it may be a mutant, variant, derivative or allele of this sequence. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1 but which encodes a polypeptide with the same amino acid sequence (e.g., +H₃N-KCNTATCATQRLANFLVHSSNNFGAILSSTNVGSNTY-CONH₂; SEQ ID NO: 2).

On the other hand, the encoded polypeptide may comprise an amino acid sequence which differs by one or more amino acid residues from the amino acid sequence shown in SEQ ID NO: 2. A nucleic acid encoding a polypeptide which is an amino acid sequence mutant, variant, derivative or allele of the sequence shown in SEQ ID NO: 2 is further provided by the present invention. Nucleic acid encoding such a polypeptide may show greater than 60% identity with the coding sequence shown in SEQ ID NO: 1, greater than about 70% identity, greater than about 80% identity, greater than about 90% identity or greater than about 95% identity.

The present invention provides a method of obtaining a nucleic acid of interest, the method including hybridization of a probe having part or all of the sequence shown in SEQ ID NO: 1 or a complementary sequence, to target nucleic acid. Successful hybridization leads to isolation of nucleic acid which has hybridized to the probe, which may involve one or more steps of polymerase chain reaction (PCR) amplification.

Such oligonucleotide probes or primers, as well as the full-length sequence (and mutants, alleles, variants, and derivatives) are useful in screening a test sample containing nucleic acid for the presence of mutants or variants of an amyloid forming polypeptide, the probes hybridizing with a target sequence from a sample obtained from a cell, tissue, or organism being tested. The conditions of the hybridization can be controlled to minimize non-specific binding. Preferably stringent to moderately stringent hybridization conditions are used. The skilled person is readily able to design such probes, label them and devise suitable conditions for hybridization reactions, assisted by textbooks such as Sambrook et al (1989) and Ausubel et al (1992).

In some preferred embodiments, oligonucleotides according to the present invention that are fragments of the sequences shown in SEQ ID NO: 1, are at least about 10 nucleotides in length, more preferably at least 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of a sequence encoding an amyloid forming polypeptide variant.

### B. Proteins

A full-length amyloid forming polypeptide of the present invention may be prepared in a variety of ways, according to known methods. The protein may be purified from appropriate sources. This is not, however, a preferred method due to the low amount of protein likely to be present in a given cell type at any time. The availability of nucleic acid molecules encoding amyloid forming polypeptides enables production of the protein using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of an amyloid forming polypeptide may be produced by expression in a suitable prokaryotic or eukaryotic system. For example, part or all of a DNA molecule, such as SEQ ID NO: 1, may be inserted into a plasmid vector adapted for expression in a bacterial cell, such as E. coli. Such vectors comprise regulatory elements necessary for expression of the DNA in a host cell (e.g. E. coli) positioned in such a manner as to permit expression of the DNA in the host cell. Such regulatory elements required for expression include promoter sequences, transcription initiation sequences and, optionally, enhancer sequences.

The amyloid forming polypeptide produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

Some amyloidogenic peptides and polypeptides, like IAPP, Abeta, and others, can not at present be expressed recombinantly and thus are prepared via chemical syntheses using so-called inteins or FMOC or BOC chemistries. Methods of chemical systhesis of amyloidogenic polypeptides are known in the art as described in, for example, Abedini and Raleigh. Org. Lett. 2005 Feb. 17; 7(4):693-6, which was the first to demonstrate chemical synthesis of IAPP by FMOC chemistry, and Williamson and Miranker 2007) Protein Sci. 16(1):110-7), which includes a description of synthesis of IAPP by inteins. The entire contents of these references are incorporated herein by reference in their entireties.

Amino acid sequences of amyloid forming polypeptides and peptides thereof are set forth herein.

The amyloid forming polypeptides of the invention, prepared by the aforementioned methods, may be analyzed according to standard procedures. For example, such proteins may be subjected to amino acid sequence analysis, according to known methods.

Polypeptides which are amino acid sequence variants, derivatives or mutants are also provided by the present invention. A polypeptide which is a variant, derivative, or mutant may have an amino acid sequence that differs from that given in SEQ ID NO: 2 by one or more of addition, substitution, deletion and insertion of one or more amino acids. Preferred such polypeptides have amyloid forming polypeptide function, that is to say have one or more of the following properties: the ability to form toxic intermediates during the course of the fibrillazation process; the ability to aggregate to form mature fibrils; and immunological cross-reactivity with an antibody reactive with the polypeptide for which the sequence is given in SEQ ID NO: 2; and sharing an epitope with the polypeptide for which the sequence is given in SEQ ID NO: 2 (as determined for example by immunological cross-reactivity between the two polypeptides).

A polypeptide which is an amino acid sequence variant, derivative or mutant of the amino acid sequence shown in SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids. For amino acid “homology”, this may be understood to be identity or similarity (according to the established principles of amino acid similarity, e.g., as determined using the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used including without limitation, BLAST (Altschul et al. (1990 J. Mol. Biol. 215:405-410); FASTA (Pearson and Lipman (1998) PNAS USA 85:2444-2448) or the Smith Waterman algorithm (Smith and Waterman (1981) J. Mol. Biol. 147:195-197) generally employing default parameters. Use of either of the terms “homology” and “homologous” herein does not imply any necessary evolutionary relationship between the compared sequences. The terms are used similarly to the phrase “homologous recombination”, i.e., the terms merely require that the two nucleotide sequences are sufficiently similar to recombine under appropriate conditions.

A polypeptide according to the present invention may be used in screening for molecules which affect or modulate its activity or function. Such molecules may be useful for research purposes.

### Uses of Amyloid Forming Polypeptides and Nucleic Acid Sequences Encoding Same

Amyloid forming polypeptides are useful in the methods and assays described herein which are directed to screening to identify modulators of toxic amyloid precursors (i.e., toxic intermediates) generated during the course of the fibrillization process and/or aggregation of amyloid forming polypeptides to mature fibrils. Such modulators may inhibit toxicity of amyloid precursors and/or may also alter the ability of amyloid forming polypeptides to self-aggregate.

### A. Amyloid Forming Polypeptide-Encoding Nucleic Acids

Amyloid forming polypeptide-encoding nucleic acids may be used for a variety of purposes in accordance with the present invention. Amyloid forming polypeptide-encoding DNA, RNA, or fragments thereof may be used as probes to detect the presence of and/or expression of genes encoding homologous proteins. Methods in which amyloid forming polypeptide-encoding nucleic acids may be utilized as probes for such assays include, but are not limited to: (1) in situ hybridization; (2) Southern hybridization (3) northern hybridization; and (4) assorted amplification reactions such as PCR. With respect to the present methods, nucleic acids encoding amyloid forming polypeptides are typically used to express amyloid forming polypeptides for use in the screening assays and methods described herein.

### B. Amyloid Forming Polypeptides

Purified amyloid forming polypeptides, or a variant, derivative, or fragment thereof, produced via expression of amyloid forming polypeptide-encoding nucleic acids of the present invention may be used to advantage in assays and methods directed to identifying modulators of toxic amyloid precursors (i.e., toxic intermediates) generated during the course of the fibrillization process and/or aggregation of amyloid forming polypeptides to mature fibrils, as discussed above.

From the foregoing discussion, it can be seen that amyloid forming polypeptide-encoding nucleic acids and amyloid forming polypeptide expressing vectors can be used to produce large quantities of amyloid forming polypeptide for use in the assays and methods described herein.

The present inventors have made the surprising discovery that IAPP toxic intermediates generated during the course of IAPP fibrillization are the effector of IAPP-mediated islet cell toxicity. Accordingly, the present findings reveal a novel target (i.e., toxic intermediates generated during the course of fibrillization) that are useful in the identification of novel modulators that may be used to advantage in the treatment of subjects with T2D and T1D, particularly those subjects who are recipients of islet transplants. In light of the above, methods are presented wherein targeting the activity of these toxic intermediates is envisioned. In accordance with the results presented herein, modulators of cellular toxicity of toxic intermediates identified herein may also modulate aggregation of amyloid forming polypeptides.

The novel findings of the present inventors, therefore, present new applications for which amyloid forming polypeptide nucleic and amino acid sequences and compositions thereof may be used to advantage. Such utilities include, but are not limited to, various screening assays and methods as described herein. Also described is a kit comprising amyloid forming polypeptide nucleic and/or amino acid sequences, amyloid forming polypeptide self-aggregation compatible buffers, and instruction materials.

### Assays and Methods

In one embodiment, the screening assay is a modification of the tryptophan fluorescence quenching assay described in the Methods Section. In brief: Toxic h-IAPP oligomers are produced in vitro as previously described herein. Toxic h-IAPP oligomers are added to a plate, such as a 384 well plate, followed by equimolar addition of small molecules and sRAGE. The fluorescence of the mixtures is measured in a fluorescent plate reader (280 nm excitation and 350 nm emission). Background fluorescence from buffer and IAPP peptides is anticipated to be negligible. The fluorescence quantum yield reported for each well will be an average of 20 reads over 20 seconds (2.5 nm bandwidth and 1 second integration time) repeated in triplicate. The quenching of tryptophan fluorescence will indicate binding of a ligand to sRAGE (the ligand could be either h-IAPP or the small molecule). No change in fluorescence will indicate inhibition of h-IAPP binding to sRAGE by the small molecule. Control experiments that measure the fluorescence of individual small molecules by themselves and in the presence of sRAGE will identify those molecules that bind sRAGE and elicit false negative hits. Final solution conditions will contain 16 mM tris HCl (pH 7.4). The peptide concentrations for the kinetic assays will be 20.1 μM h-IAPP or rat IAPP (negative control) and 20 μM sRAGE).

In a particular embodiment, the library of small compounds or agents can be purchased from a commercial vendor. Such libraries are known to those skilled in the art and are used routinely. An exemplary library of small molecules can be accessed at the worldwide web site provided by chembridge via screening libraries and more particularly, via diversity libraries (e.g., chembridge.com/screening_libraries/diversity_libraries/index.php?PHPSESSID=950cc192632a72a4290423c77aa40261#DIVERSet).

The present inventors have determined that the fluorescence read out in their in vitro assay may be used for high throughput screening purposes. Cell based assays are used as second line screening assays after “hits” have been identified in primary screens. Such cell based assays are described herein. The Alamar blue cytotoxicity assay is an exemplary cell based assay that can be used as a confirmatory screening assay.

### Additional Assays and Methods

The interaction of oligomeric precursors and candidate agents can also be detected using Tyrosine fluorescence or the florescence of non-genetically coded amino acids, these include p-cyanoPhenyl Alanine and para-ethynylphenyl-alanine. The interaction can also be detected by monitoring changes in the fluorescence intensity or fluorescence anisotropy of dye molecules which have been covalently attached to sRAGE or IAPP via an engineered Cys residue or to the amino group of a lysine side chain or to the n-terminal amino group. The dyes may also be attached through an unnatural amino acid such as azido phenyalanine or azido homo alanine or via an amino acid which contains an Alkyne group. Such approaches related to CLICK chemistry. Suitable dyes included any and all ALEXA dyes, any and all Rhodamine dyes and any other useful dyes such as Dansyl, etc.

The interaction could also be detected by, but not limited to, spectroscopic techniques which lead to a change in signal between the bound and Free State. Such techniques include fluorescence emission spectroscopy, measurement of fluorescence anisotropy, Fluorescence resonance energy transfer, Absorbance spectroscopy, FRET, CD, and IR. The interaction may also be detected by surface plasmon resonance (Biacore). The interaction may also be detected by isothermal titrating calorimetric methods or by thermal shift assays.

The stability of sRAGE can also be measured in the presence and absence of the “drug” identified by the screen. If the drug binds sRAGE, then sRAGE stability will increase. Stability is measured by deducing the temperature at which the protein unfolds. This is achieved by adding a dye which binds to unfolded aggregated proteins. In other words, one would detect the temperature at which sRAGE unfolds by seeing an increase in the fluorescence of the dye. One would then follow by repeating the experiment again in the presence of the “drug”. An increase in the melting temperature would indicate whether the drug bound to sRAGE. For the sake of clarity, this assay is not an activity assay, but it could be used to find compounds that bind to sRAGE.

A cell used to produce sRAGE can be a bacterial cell, a mammalian cell, an insect cell, or a yeast cell. A bacterial cell used for the production can be an Escherichia coli cell, a Bacillus cell, a Salmonella cell, a Lactobacillus cell, a Lactococcus cell, a Streptomyces cell, a Streptococcal cell, or a Corynebacterium cell. A yeast cell which could be used in the method(s) of production can, for example, be a Pichia cell, a Saccharomyces cell. Mammalian cells which could be used to produce sRage include, but are not limited to, monkey cells, human cells, mouse cells, a HeLa cell, CHO, Jurkat, HepG2, H1299, HEK293 cells or NIH 3T3 cell or hamster cells.

Methods for making sRAGE are known in the art and are described, for example, in Park et al, (1998). Nat Med 4(9):1025-31.

Soluble RAGE Domains Amino Acid Sequences:
V-Domain (residues 23-116; SEQ ID NO: 19):
AQNITARIGEPLVLKCKGAPKKPPQRLEWKLNTGRTEAWKVLSPQGGG
PWDSVARVLPNGSLFLPAVGIQDEGIFRCQAMNRNGKETKSNYRVR
C1-Domain (residues 124-221; SEQ ID NO: 20):
PEIVDSASELTAGVPNKVGTCVSEGSYPAGTLSWHLDGKPLVPNEKGV
SVKEQTRRHPETGLFTLQSELMVTPARGGDPRPTFSCSFSPGLPRHRA
LR
C2-Domain (residues 227-317; SEQ ID NO: 21):
PRVWEPVPLEEVQLVVEPEGGAVAPGGTVTLTCEVPAQPSPQIHWMKD
GVPLPLPPSPVLILPEIGPQDQGTYSCVATHSSHGPQESRAVS

### Hydrophobic Patch on the V-Domain:

a large highly conserved slightly recessed hydrophobic patch extends, but is not limited to residues Ile-26, Ala-28, Pro-33, Leu-34, Val-35, Leu-36, Leu-49, Trp-61, Val-63, Leu-64, Trp-72, Val-75, Val-78, Leu-79, Pro-80, Phe-85, Leu-86, Pro-87, and Val-89 as well as the hydrophobic parts of the Lys-37 and Tyr-113 side chains.

### Basic Patch:

The residues involved in constructing the positively charged surface include Arg-29, Lys-37, Lys-39, Lys-43, Lys-44, Arg-48, Lys-52, Arg-98, Arg-104, Lys-107, Lys-110, Arg-114 and Arg-116 from domain 1 as well as Arg-216 from domain 2.

The above information was determined based on the following: worldwide web uniprot site subdirectory uniprot/Q15109#section_comments; Park et al. (2010) JBC 285(52), 40762; and Koch et al. (2010) Structure 13; 18(10), 1342. The contents of Park et al. and Koch et al. are incorporated herein by reference in their entireties.

Based on results set forth herein, the present inventors have shown that the V-domain is critical for IAPP binding and thus, is an exemplary functional fragment of IAPP. Proper protein folding may, however, be required for IAPP-sRAGE binding and thus, a functional fragment may further comprise the V-domain be intact with the C1 domain and/or may require the fully intact V-C1-C2 domain. It may also be possible that IAPP may bind to the C2 domain.

Protocol: sRAGE Production, Purification and Characterization

### I. Production

### Revive New SF9 Cells:

1. Thaw vials from −80° in hand until ½ ice, ½ medium
2. Pipette into 20 mL 10% FBS medium in 50 mL tube
3. Spin down, 800 rpm, 7 minutes
4. Pipette off supernatant
5. Resuspend in 12 mL FBS medium in tube
6. Pipette into T-75 flask, leave in hood for 20 minutes until cells settle to bottom (attach)
7. Put into large incubator, leveled shelf

### Alternative Steps to the Above:

5. Resuspend in 40 to 50 mL FBS medium in a small spinner flask
6. Put spinner flask in the 27° C. incubator, make sure it is spinning
7. When the cells reach a high density (see below), double the medium volume and transfer cells to a bigger spinner flask

### Culture New Cells in T-75 Flask (12 mL Medium):

Add medium to spinner flasks: 500 mL bottle of Grace Insect Medium plus 60 mL FBS (10-12% FBS) plus 0.6 mL Gentromycine (antibiotic). Change ½ of medium volume every 2-3 days (every 3-4 days maximum). NOTE: 50-60% coverage=one more day. 80-90% coverage=ready to split/use).

### Determine Confluency of Cells in Spinner Flasks by Counting:

1. 900 uL cell culture, 100 uL Trypan Blue (10 uL per homocytometer), aspirate soup and resuspend cells before plating.
2. Evaluating cells: ready for transfection at 2.5-3.5×10⁶ density (NOTE: 160 cell count per full center grid of homocytometer is also a good number). Alternatively, mix a 1:1 mixture of 1 ml cell solution and Trypan blue in a 35×10 mm cell culture dish. Check under microscope.

### Viral Transfection:

1. Virus in 4° C. cold room, labeled “SR” with date in tube
2. 25 mL virus per 500 mL cell culture
3. Incubate overnight before change medium.
Transfer Transfected SF9 Cells to Serum-Free Medium in Spinner Flasks for sRage Expression Culture:
1. Pipette 500 mL cell culture from one spinner flasks into ten 50 mL tubes
2. Spin down 800 rpm for 7 minutes
3. Pipette out supernatant
4. Resuspend cells in 10 mL serum-free medium in the tube
5. Pipette cells back to spinners and fill to 500 mL with serum-free medium

### Cell Harvest:

1. Pour 500 mL cell culture into (10) 50 mL tubes
2. Spin down 1500 rpm for 7 min.
3. Pour supernatant into glass bottles, label as sRAGE, date, store in 4° C. cold room.

### Virus Harvest:

1,500 rpm 7 min. Do amplification before use for production. (3 to 4 days after transfection, without medium change).
Note: Cell replication time (to double the cell amount) varies. Check/count cells under a light microscope to ensure good quality and density before splitting/transfecting.
II. sRAGE Purification and Characterization
Dialyze Baculovirus Medium Containing sRAGE (Stored in Cold Room):
1. Prepare 2 large beakers with 400 mL 10×PBS plus 3.6 L dH₂O=4 L 1×PBS
2. Cut (4) 2.5 foot-lengths of Spectra/Por Dialysis Membranes (See Supplies below for product order info) and soak in PBS/dH₂O
3. Tie off one end with two overhand knots, then add buffer and check for leakage. Using funnel, fill with approximately 500 mL of solution.
4. Tie off with two overhand knots and place two tubes per Beaker, into 4° C.
5. Refresh buffer two more time over 2 days.

### Filter Serum-Free Medium Prior to FPLC Purification:

Change 0.45 micron filter approx. every 500 mL of serum-free medium.

### FPLC Procedure:

1. Place filtered serum-free medium on ice
2. Mount purification column red-top up (GE Healthcare HiTrap SP HP 5 mL cation-exchange)
3. Prepare FPLC elution buffers A & B:
a. Buffer A=100 mL 1×PBS+3 g NaCl+up to 1000 mL DDI H₂O (FILTERED)
b. Buffer B=100 mL 1×PBS+35 g NaCl+up to 1000 mL DDI H₂O (FILTERED)
4. Wash FPLC lines with warm ddH₂0 if system is filled with EtOH (alcohol reservoir should be full)
5. Prime pump with syringe, check for clarity of liquid
6. TIMING: Set-up: 30 min.; Clean-up: 45 min.; Sample loading: 5 mL/min flow rate results in 2 hours/500 mL serum-free medium; Elution gradient: 0 to 90% Buffer B in 90 min.

### Method Editor:

7. UNICORN→System Control→Run→program file: AndiSophi
8. Define fractionation tubes and method:
a. Variables:
i. Wash Inlet A: off
ii. Wash_Inlet B: off
iii. Flow rate: 5 ml/min
iv. Column Pressure: 0.6 to 1.1 max
v. Sample Pressure: 0.6 to1.1 max
vi. Average time UV: 5.1
vii. Start Conc B: 0.0
viii. Sample volume: 500 mL to 1000 mL per run (per column)
ix. Pressure Alarm: no greater than 1.1
x. Sample collection in tubes: 2 mL
xi. Gradient Length: 90 (this means 90 column volumes×5 mL column volume=450 mL (elution gradient is 0-90% buffer B in 450 mL, where flow rate is 5 mL/min).
b. Monitor Run Pressure (0.6 or lower) If alarm is triggered:
i. Change alarm to higher limit (no greater than 1.1)
ii. Change filter on M-925 unit (black box on windowsill)
iii. Wash with alcohol, detergent, etc.
iv. Call Service Dept.
c. Finish:
i. Remove column and connect lines with black spacer connection
ii. Wash both direct-loading sample line and buffer lines with 200-400 mL ddH₂0, 20 mL/min rate.
ii. Save program and shut down FPLC
9. Maintaining the FPLC with a weekly wash procedure
a. Sample Pump=“DirectLoad”
i. Manual→Pump→DirectLoad
ii. 500 mL ddH20, 10-20 mL/min rate
iii. 500 mL detergent (10% dilution w/ddH20) 10 mL/min
iv. 500 mL ddH20, 500/20
v. 200 mL 20% EtOH solution 10 mL/min
vi. Leave alcohol in system for next time, be sure to do ddH20 wash to remove before next run.
b. Buffer Pump=“PumpWash”
i. Manual→Pump→PumpWash
ii. ˜200 mL ddH20 (automatic settings, though)
iii. ˜200 mL 20% EtOH solution (auto)
iv. Leave EtOH in system until next time, be sure to wash with ddH20 after.

### FPLC Elution Profile:

The FPLC purification profile should show separation of two well-resolved sRAGE peaks. Peak A corresponds to full length sRAGE (36,254 Da), while Peak B corresponds to a lower molecular weight, C-truncated sRAGE variant (34,710 Da). The Peak A elution maxima is usually seen around 40% Buffer B, while the Peak B elution maxima is usually around 50% Buffer B. For mass and amino acid analysis see Mass Spectroscopy section below.

After FPLC Purification, Dialyze sRAGE Peak Eluents (×3) to Remove NaCl and Exchange into 1×PBS (or H2O+0.001% Acetic Acid Before Lyophilization):
1. Transfer collected FPLC fractions to dialysis cassettes (5000 mw cut off) using 10-50 mL syringe.
2. Place cassettes into a large beaker containing a stir bar and either:
a) 2000 mL H₂O+0.001% acetic acid (pH 4.5-5.0) or
b) 1×PBS: 100 mL 10×PBS+900 mL dH₂0
3. Cover beaker with seran wrap and place on stir plate in 4° C.
4. Replace beaker solution with fresh (a) or (b) every 3 hrs and finish with one last overnight exchange at 4° C.
NOTE: Alternatively, use centricon to desalt and concentrate protein.
sRAGE Lyophilization:
1. Remove desalted sRAGE solution from dialysis cassettes (or centricon) using syringe and transfer to 50 mL tubes.
2. Freeze solutions in 50 mL tubes via liquid N₂
3. Remove cap of tube with frozen solution and cover with a folded sheet of kimwipe using rubber band to hold kimwipe in place.
4. Place sample on lyophilizer vacuum trap until complete sublimation and protein is a dry, white powder.
5. Store dry sRAGE at −80 C (make sure protein is in a cold, dry environment, such as a vacuum sealed desiccator filled with desiccant).

### Mass Spectroscopy:

The identity of FPLC peaks A and B can be confirmed by molecular weight analysis using MALDI-TOF MS. Analysis of FPLC fractions obtained from Peak A and Peak B confirm the presence of 2 heterogeneous species with molecular weights of 36254.90 and 34710.29 Da, respectively. The molecular weight distribution of the two sRAGE peptides are similar and suggest heterogeneity in GlcN and GlcNAc glycosylation. For MALDI-TOF MS, myoglobin was used as an internal standard. Salt was removed from samples prior to ionization by running samples through a C4 zip tip. Samples were treated with sinapinic acid and standard BSA methods. C-terminal truncation of the lower molecular weight sRAGE (Peak B) was verified by MS/MS. For MS/MS, Coomassie-stained gels were reduced with DTT, alkylated with iodoacetamide, and digested with trypsin.

Any of the sRAGE protein variant can also include a chemical modification selected from the group consisting of amidation, lipidation, glycosylation, pegylation, and combinations thereof. The modification may be generated in vivo in cells or in vitro by chemically modifying the protein.

### Therapeutic Uses of Modulators Identified Herein

The invention provides for treatment of amyloidosis diseases (e.g., diabetes) by administration of a therapeutic agent or compound identified through the above described methods. Such compounds include but are not limited to proteins, polypeptides, peptides, protein or peptide derivatives or analogs, peptoids, antibodies, nucleic acids, and small molecules.

The invention provides methods for treating subjects/patients afflicted with an amyloidosis disease comprising administering to a subject an effective amount of an agent or compound identified by methods of the invention. In a preferred aspect, the agent or compound is substantially purified (e.g., substantially free from substances that limit its effect or produce undesired side-effects). The subject is preferably an animal, including but not limited to animals such as cows, pigs, horses, chickens, cats, dogs, primates, etc., and is preferably a mammal, and most preferably human. In a specific embodiment, a non-human mammal is the subject.

Formulations and methods of administration that can be employed when the agent or compound comprises a nucleic acid as described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into a localized site that is the predominant pathological site of the amyloidosis disease, such as, for example, the pancreas.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., a target tissue or tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### Therapeutic Uses of Nucleic Acid Sequences Encoding Modulators Identified Herein

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, a nucleic acid encoding a modulatory agent identified using the methods described herein is incorporated into an expression vector that expresses the modulatory agent in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In a particular embodiment, “ex vivo gene therapy” can be used to genetically engineer islets intended for use as transplants to express soluble RAGE (sRAGE). In a more particular embodiment, the genetically engineered islets express human sRAGE or sub-fragments thereof comprising the V-domain of sRAGE.

The following is a brief protocol for isolating islets for transplant. The pancreas is removed from the donor and digested to extract the islets. Fully intact islets, which contain functional beta cells and alpha cells, etc., are then cultured. Detailed protocols for isolating islets are known in the art. See, for example, Potter et al. Proc Natl Acad Sci USA. 2010 Mar. 2; 107(9):4305-10; Plesner et al. J Transplant. 2011; 2011:979527. Epub 2011 Dec. 22, the entire contents of each of which is incorporated herein by reference.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a particular embodiment, pancreatic islet cells are delivered surgically, e.g., via catheter or laparoscopic surgery. In another embodiment, epithelial cells are injected, e.g., subcutaneously. In yet another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to pancreatic islet cells, neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a nucleic acid sequence encoding a modulatory agent or compound of the invention may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

### Pharmaceutical Compositions

The present invention also provides pharmaceutical compositions. Such compositions comprise a therapeutically effective amount of an agent or compound, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of an amyloidosis disease can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

Protein based drugs are often formulated with “inert” additives such as polymers. Accordingly, pharmaceutical compositions comprising, e.g., sRAGE and at least one pharmaceutically acceptable carrier may further comprise at least one polymer selected from the group consisting of alginates, chitosan, collagen, fibrins, methoxy poly(ethylene glycol), polyanhydrides, poly(caprolactone), poly(ethylene oxide), poly(lactic acid), poly-lactide-co-glycolide (PLGA), poly(ortho esters), polyethylene vinyl-co-acetate (EVAc), polyethylene glycol (PEG), polyester-PEG triblock copolymers, polyphosphazenes, poly[(sebacic-co-(ricinoleic acid)], ricinoleic acid, silicone, and multiple component combinations of the above.

Pharmaceutical proteins may be artificially post-translationally modified with inert, covalently linked polymers such as PEG to slow clearance and increase “bioavailability”.

Also encompassed herein are modified forms of sRAGE, such as various post-translationally modified forms thereof (e.g., glycosylated forms). Modified variants of sRAGE are also envisioned herein. Accordingly, any of the sRAGE protein variants can also include a chemical modification selected from the group consisting of amidation, lipidation, glycosylation, pegylation, and combinations thereof. The modification may be generated in vivo in cells or in vitro by chemically modifying the protein.

A nucleic acid sequence for Abeta is presented below:

```
        1 ggggcggggc tggcggcgcc ggcgcagccc gggggcggcg ggaggaggag gtggcggcgg
      61 tggcgctggg agctcctgtc accgctgggg ccgggccggg cgggagtgca ggggacgtga
    121 gggcgcaagg gccgggacat ggggcccgcc agccccgctg ctcgcggtct aagtcgccgc
    181 ccgggccagc cgccgctgcc gctgctgctg ccactattgc tgctgcttct gcgcgcgcag
    241 cccgccatcg ggagcctggc cggtgggagc cccggcgcgg ccgaggtgag gccgggccgg
    301 gtcctggggg atgggggaag gggcgggacc gggtctctgg acgccggcgc ggacatgtcc
    361 agggcagaaa gcgcggtctt tccagccagg tggtcagccc ccaggcgccc ccaatcacat
    421 ttatgaaccc agggttccag gccccagctc ccccatcatg cgacgtccca gccccctccc
    481 atctcgagca taggaactgg tctattcaga gcccctggtc ccagaagtcc agccccctct
    541 ccagacccag gtgactcggc cccaaccccc tcccgcctgg acataggacc caccaagcag
    601 cgaggcattt agatccaata atccagaccc cttgtattct ctggacccat atggaggccc
    661 ttgcagcctc ccaggaccca ggagtccagt ccttcagtca ccacccaccc caaccagatg
    721 tagctctcca gtcctcaagg acctggtgtc caggactgta ggcccctgaa gccaggcctt
    781 gtcagctttg catcctgcaa cgggagcctg agcaagggat ggagggagga ggggccagaa
    841 ctcctgggtt ctggcctcct cctccgcgat tcaggtttaa ccccttcggg ctccagagcg
    901 gctgcgctgg ggtgggggcg gagtctgtct ccgcggcaac aaggcagaaa gaatcccggg
    961 ggacccaggt cgccatagca acgggagcgc tggggcgccc ccgccctacg ggagctgttt
  1021 cccagggaac ggtgcctcca tggaggcggt gtgcggtgct tgggggaggg ggctggtgct
  1081 gggggtctcg gtcctaggga gcaaagaacc aggggaccct catgccaacg ccccccgagc
  1141 cctcactgtc ctttccactt ccatccaggc cccggggtcg gcccaggtgg ctggactatg
  1201 cgggcgccta acccttcacc gggacctgcg caccggccgc tgggaaccag acccacagcg
  1261 ctctcgacgc tgtctccggg acccgcagcg cgtgctggag tactgcagac aggtgggcgg
  1321 ggccgaacgg gagaggcggg gccgcccata gaaagctaga cttgaaaaag gcgtggtcca
  1381 gggtgctgcg cgatctaagg cgtggaggct ggggggcgtg gccaataaag aggcgcaact
  1441 atgctagggg caggggacct gttttgagat actaagtcag gaaaagggga gagccgcgag
  1501 atagccagag aggaagtgga atttaggaat ctggtggtct ttgtaaagag tagaggtgta
  1561 ggggggagtg gcgaaaggat aggcggggct aagacagaaa gagaccttaa ggaccagcaa
  1621 gatggggaaa ggggtggagc ccaatgagag cgcggagagc tgggggggcg tggccatgaa
  1681 aagacaaatt tataacggga agggagagtt ttggagaggc ggaatagagg aaaaggcggg
  1741 gcctaaagga gggtgagacc tttggggaga cgaatctgac tgcggggagg ggtgaccaga
  1801 gaggtgggct tagagggacc ttcagaaaga aacagcacag gaaaagagat agggcttaaa
  1861 gatgacggga cttttaaggg aaaactgcta gtgggcgtgg ccaatgagca caaggagctt
  1921 ggatatctaa ggctggtgct agggagaagc agggcctagg gaagcgatgt cctcatgaat
  1981 actagagcct tgaaaacgga cctggccggg cgcggtggct cacgcctgta atcgcagcac
  2041 ttggggaggc cgaggcaggc ggatcacctg aggtcagaag ttcgagacca gcctggccaa
  2101 cacggcgaaa ctccgtctct actaaaaata caaaaattag cctggcatgg tggtgcgtgc
  2161 ctgtaatccc agctactcag gaggctgaga caggagaatc gcttgaacct gggaggagga
  2221 ggttgcagtg agccgagatt gtaccattcc actccagcct gggcgacaag agcaaatctc
  2281 cgtctcaaag aaagaaagaa agagggagaa agaaagagaa aagggacctg actactggag
  2341 aggggtggct ggcaggggcg gggcagtggg ctgattgccc ccatctgatc cccccagatg
  2401 tacccggagc tgcagattgc acgtgtggag caggctacgc aggccatccc catggagcgc
  2461 tggtgcgggg gttcccggag cggcagctgc gcccaccccc accaccaggt tgtgcccttc
  2521 cgctgcctgc gtgagtccca ggcggggaga ggggaactga ggtgggagtt tctgaggggc
  2581 aaggttctga gcccctctct caggcctaca ttaaggggct gggtgcttgt gtcctaagtg
  2641 gggcagagaa gcctctgagg ataaaatatc tggattctga ggagggtggg gttggtggct
  2701 ataggaggat ctcaccctgg tgtcccgtgc ttccccagct ggtgaatttg tgagtgaggc
  2761 cctgctggtg cctgaaggct gccggttctt gcaccaggag cgcatggacc aatgtgagag
  2821 ttcaacccgg aggcatcagg aggcacagga ggtcaggacg ttggcccacc cgtccccagc
  2881 ccccacaacc caggaactgg gacctctaac accctccgcc accagaaccg aggagtctgg
  2941 gccaccagca tcctcttcgc acttgggatc taagaatttc atcccccaac cccttcctct
  3001 agaagcagga atccaggctc ccagcctcat caacccccaa ccctggcagc ccagttcccc
  3061 atctaccccc tcccatccca caatcctggc atctgggccc actcttccta caggcctgca
  3121 gctcccaggg cctcatcctg cacggctcgg gcatgctctt accctgtggc tcggatcggt
  3181 tccgtggtgt ggagtatgtg tgctgtcccc ctccagggac ccccgaccca tctgggacag
  3241 cagttgggtg agtgggaggg aaccctccat gcccatctca aggttcctga ggcaggggat
  3301 ggaagcctgg gagcccaggc ctgggttctt actgcctggg tcctctcctg ctccctcagt
  3361 gacccctcca cccggtcctg gcccccgggg agcagagtag agggggctga ggacgaggaa
  3421 gaggaggaat ccttcccaca gccagtagat gattacttcg tggagcctcc gcaggctgaa
  3481 gaggaagagg aaacggtccc acccccaagc tcccatacac ttgcagtggt cggcaaaggt
  3541 gaggcagtct ctgaacccct ggggcctctc caccatagag ggagaaagat ctgggggagt
  3601 cttgctgggg ggtgtctttg ggaggggcct ataggggaaa ggcccaactg aggagaaaag
  3661 acgagagtat ctttggataa aatagaagta gaagggctaa cctgccaagg gagggggtgg
  3721 tttgggggta cttgggagta gaggggccat tgggtaggtc ttgaggatca tttcaggaaa
  3781 gcttggaaga tggtgtaatg gattcctaag ctttgcaaga acaggcccag tccagaacta
  3841 catctcccat aatgccaggc agcagcggtg gctaaactgg gtgcatgatg gtctccagtg
  3901 cactctagga aatgtggttc tctaggtaga aaaggcgacc tggaggtggg ctgcagactg
  3961 acctcctgat ccctggtctt gcagtcactc ccaccccgag gcccacagac ggtgtggata
  4021 tttactttgg catgcctggg gaaatcagtg agcacgaggg gttcctgagg gccaagatgg
  4081 acctggagga gcgtaggatg cgccagatta atgaggtgat aatactgggg gccccaggac
  4141 cccctacagt acagagctcc ctaaatacca ggaaattcct ccaggacaca ttgatactac
  4201 ctccaaaggc tccctaagcc cctttgacct tgagctctca acaccacccc ctaagatggc
  4261 cagagatcca tggcccttct agaatcccac tgagacgcta ccaggttctc tggaaactct
  4321 ggtctatggt actctttcac tttattggtt tttttttttt tcttttgttg ttgttgttgt
  4381 gacggagttt cgctcttaac acccaggctg gagtgcaatg gtgcgatctc ggcccactgc
  4441 aacctctgcc tcccgggctc cagcgattcc ccttcctcag tctcctgagt agctgggatt
  4501 acaggcaccc accaccacgc ccggctaatt tttgtatttt tagtagagac agggtttcac
  4561 catgttggcc aggatggtct tgaactcccg gcgggaggag atccacccgc ctcggcctcc
  4621 caaagtgctg ggattacagg catgagccac cacgcctggc ctctctttca ctttaaactc
  4681 cttctggatc ttccctcttg ggaacccagg agccagcgag acttaaggga tctggggcct
  4741 ttaaatcttt tttttttttt ttttttgaga cagagtttcg ctctgttgcc caggctagag
  4801 tgcagtgacg tgatctccca ctcactgcaa gctccacctc ctgggttcac gccattctcc
  4861 tgcctcagcc tcccgagtag ctgggactac tggtacccac cacagcgccc agataatttt
  4921 ttctgttttc agtagagaca gggtttcacc atgttagcca ggatggcctc aatctcctga
  4981 ccttgtgatc cacccacctc ggcctcccaa agtgctggga ttacaggcat gagccactgc
  5041 gcccagccat ggggcttcta aaatcttaaa gaggggttgg gggacttgcc aggtggatca
  5101 gggtggattc tgggatcctg aagctcccct ccctatgcag gtgatgcgtg aatgggccat
  5161 ggcagacaac cagtccaaga acctgcctaa agccgacaga caggccctga atgaggtagg
  5221 acagccccag tgggtcctac tcatgcctgt ccaccacctg gagcacactc agtttcacct
  5281 ggctctggct gtgccctgcc catccagttc caccccttcc cacctatctc agcctttcct
  5341 ggccccatgc ctacatgcag ctctgcccct cttagccgtc atctgacctg acactgctct
  5401 cctccccaga ttggccatat tcggccccat ctacagactt gacttgcctc tcagggctgg
  5461 ctctggagtc ctgtcccaag ccagggcctc tgcagatgca gccagggcct tcttggtctc
  5521 tctttgatgc atttatgtct ctatcaggcc ccgccccctg attctggctc tgctgggcca
  5581 atctcacctt tattaacctg acctacccca tggagacccc actcatgtta gcccccattc
  5641 cagctctttg tcccacccct atcgtgtcat ttatacacag cctgtctcca gtttgaccct
  5701 gcccaggcca ggagccctgc aaggctttgt ccctttcacc ttaacattgg tcagttctgc
  5761 tcccagattg ctcccactca atcttacagt ttacatcctc acattggctc ccagtgggcc
  5821 tagtcccacc tccactctgc ctggccctgt agcccacccc ttccagtcca taacctttgg
  5881 ttctgcccag gcctggaccc ctggaacgcc ccccaacccc atgtagccct gcctttccag
  5941 gctctctttg accaggcttt gacccatctt ctcctctcct gaccctgtgc ccacccgctc
  6001 cccagcactt ccagtccatt ctgcagactc tggaggagca ggtgtctggt gagcgacagc
  6061 gcctggtgga aacccacgcc acccgcgtca tcgcccttat caacgaccag cgccgggctg
  6121 ccttggaggg cttcctggca gccctgcagg cagatccgcc tcaggtgcgg ggaccgtggg
  6181 ggcagagagc agagggtgag aagggtcagg gcgggcttgg gcatcctgtg tcccttccac
  6241 aggcggagcg tgtcctgttg gccctgcggc gctacctgcg tgcggagcag aaggaacaga
  6301 ggcacacgct gcgccactac cagcatgtgg ccgccgtgga tcccgagaag gcacagcaga
  6361 tgcgcttcca ggtgctcaca tccttccagc tcccaaatgc gccgctattc ctcagacgcc
  6421 cgcgcctcag gctcttctct tgtcccttag accctctttc tgtctcttgg accccttcct
  6481 atcccctgaa caccgcttct ctgccccttc ccagtctctc agctcagctt cctgaccctg
  6541 aaacatggac cctcacatgc tgtgtctttg acccctgctt cttggccctt ggattcctac
  6601 tccccccgcc gtcgatccta tgttctgtcc cttggatttt cactgccttt cccagaatcg
  6661 tctttttttt tttttttttt ttgagacagg ttcttgctct gtcgcccagg caggagagca
  6721 gtgtgcgatc ttggctcatt gcaacttcca cctcctgggt tcaagcaatt ctcctgcctc
  6781 agcctctcga gtagctggga ttacaggagc ctgccaccac actgggctaa tttttttttt
  6841 tttttttgac agagtctcgc tctgtttccc aggctggagt gcagtgacat gatctgggct
  6901 cactgcaacc tccgcctact gggttcaagc tattctcctg cctcagcctc ctgagtagct
  6961 gggactacag gcgggtgtca ccacatctgg ctgatttttg tatttttagt agagacaggg
  7021 tttcaccata ctggtcaggc tggtcttgaa ctcgacctca ggtgatccac ccttggcctc
  7081 ctaaagtact cggattacag gtgtgagcca ccacgcccgg ccccagctaa tttttgtatt
  7141 tttggtagac acgggtttca gcatgttggc caggctggtc ttgaactcct gacctcaggt
  7201 gatctgcctg ccttggcctc ccaaagtgct gggattacag gcgtgagcca ccatgcccag
  7261 ccagaaaccc caataacttt tgcaccaatc taatattttt agcagagaca gggttttgcc
  7321 atgttgccca ggctggtctc gaactcctga cctcaggtga tctgcccacc tcggcctccc
  7381 aaagtgctgg gattacaggc gtgagccacc atgcccggcc agaaacccca ataacttgca
  7441 ccaatctaat atttttagca gagacagggt tttgccatgt tgcccaggct agtctcaaac
  7501 tcctgacctc aggtgatctg cctacctcgg cctcccaaag tgctgggatt acaggcatga
  7561 gccaccgcgc ccggtcgaga atctccttct tgttccttga accctcttcc tgtccctcaa
  7621 cctcctttct ccataacttc acttgttttc cctggaaccc ctgttctgtg cgctcaaatt
  7681 tgaattcccc tttcctggat gttttcttcc tgtctatgaa actccattct gtgctcttga
  7741 actccaaatc ttgccttgaa ccatgtcatt tctatatgac cctccaatcc tcaatctctg
  7801 tctctggaat cccctcaaac cccactttct gttccttgga ctttattctt caatttcctt
  7861 ctcctatggc ccagttccta acccttgtac cacacatcct gtccattgca tgtgccgctt
  7921 ttcctcagtc gctattgaat tcctccttca tactgcttca gtttcctcat ctccagcctg
  7981 cattgcgcag ttcatccttc atgtccactc acccacaggt gcatacccac cttcaagtga
  8041 ttgaggagag ggtgaatcag agcctgggcc tgcttgacca gaacccccac ctggctcagg
  8101 agctgcggcc ccaaatccgt gagtgtctat taccctggct cccattacag atctctgagg
  8161 gcagatcttg actcctaaat gttgggcccc cccaatttca tttattcctc tataacaaac
  8221 agcccagacc ttagcagtga aaatcaacaa tgatttttct ttgttcatga ttctgccatc
  8281 cggtctgcgc tcagcagagt ggttctttca gtggtcttgc cagtggtcaa gcatgcagct
  8341 gtatttagct agcagatcat ctaggggctg ggagtctagc acaaatggac ctttctctct
  8401 ctccaaggaa gcgcaaggcc tctcttctcc gtggagcttc tccatgtggt ctcatcagca
  8461 gggtagctag attccctaca tggtggttta tgctctctaa gacatcacag tggaagttgc
  8521 taggtcttaa ggcttgggcc cacattctat ttgttaaagc aagttacaaa ttcagtccag
  8581 attcaaggga aggaacctat atgcataccg gaaagtgtga cctattgcag cccccacatc
  8641 tattgtgtct ttctcctgga tatctcacac ataaccctga ttctcctagt atttaagaaa
  8701 gctatcatct tgaggcgcgg tggctcacgc ctataatccc agcactttag gaggccgagg
  8761 cgggtggatc acttgaggtc aggagttcga gaccagcctg gccaacatgg tgaaaccccg
  8821 tctttactaa aaatacaaaa atcagccggg catgatgtcg cttgcctgta atcccagcta
  8881 cttaggaggc tgaggcaaga gaattgcttg aacccgggag gtggaggttg cagtgagctg
  8941 agatcgcatc attgcactcc agctgggcaa caagagtgag actctgtctc aaaaaaaaaa
  9001 aaacaaaaaa aaaacataat cttgaaactt cagcctccat ccttcctgcc agcagtgcct
  9061 ccatccagct tcccactttc tcagatcaca cttctggcta ccccacactt ggggctgact
  9121 ctgctgtctg catgatctcc cacttgctct actggtaggg tgccctccac tcacccctat
  9181 gctcactacc tcagccacct ttctgcatgt ccccctcaga ggaactcctc cactctgaac
  9241 acctgggtcc cagtgaattg gaagcccctg cccctggggg cagcagcgag gacaagggtg
  9301 ggctgcagcc tccagattcc aaggatggtg agtgagccca catatagatg accccagaca
  9361 ttagggaaca ggccccagcc taatttgtaa tcccctagag tctgagggtg tcttcaccac
  9421 cacagtgact gggagaggat gaggaggaac gtctaaggtt gcaggggcct ctgtaggatc
  9481 cccaatcctc cttcttagtc cctggaagga tgtttctcca cctttctttg ctgataccct
  9541 cctctcttca ctgttccact cccttgcttc ctctggctgc cagcagacac ccccatgacc
  9601 cttccaaaag gtgagtgtct cacagttaac cccagcctcc aaatcccact gaatccctga
  9661 acccagaagg aaacagggtc catccattgg gaacctcaga ccccctgggg tagagtttga
  9721 tgtactttcc agccccctcc tctggaccct aaagaatgag atagggccag gcgctggtga
  9781 ctcacacccg taatcctagc actttcagag gctgaggcag gaggatccct tgaggccacg
  9841 agttctagac cagcctgggc aacataatga gaccctgtac ctacaaataa tttaaaaatt
  9901 acctgggtgt ggtggggcat gtctgtagtc ccagctgctc aggaggctga cgtagaagga
  9961 tcactggagc ccaggaagtt gaggctgcag tgagctgaga tcatgccact gcactccagc
10021 ctgggtgaca gagtgagact ctgtctaaag aaaaaaaaaa agaatgagat cagacttggg
10081 ggtagggtcc acagaacaag atgctgcatc ccctgagaaa gagaagatga acccgctgga
10141 acagtatgag cgaaaggtaa gttagtcaga actgtgggct ccctaagggg aacaagatcg
10201 gggcctatat ggctgggtac gagggaggag atgctggggg cttggattcc ttgtcctgag
10261 ggaagaggga gctgaggacg tggaattgag atcctagaaa atgagagggc tgggggacgc
10321 tctcttgggc ccttgggtag gaagaagcca gtgccaggct tctgggttcc tgacacctcc
10381 tgctccccca ggtgaatgcg tctgttccaa ggggtttccc tttccactca tcggagattc
10441 agagggatga gctggtaaga ggaggaacag ccgggtacct aggggaagag accagaggtc
10501 agcggccagg ctgtgattcc caaagccaca caggaccctc aaagaagccc tctgccccat
10561 ctcctctccc tgcaggcacc agctgggaca ggggtgtccc gtgaggctgt gtcgggtctg
10621 ctgatcatgg gagcgggcgg aggctccctc atcgtcctct ccatgctgct cctgcgcagg
10681 aagaagccct acggggctat cagccatggc gtggtggagg tgagaaccat ggcgtggtgg
10741 aggtgtggga agagttcctg agcccgggtg tgggcggcct gagagacttg cgggcagtcc
10801 cgcccccgca ccacactgtc ctttccctcc cctgctcgtt gcaggtggac cccatgctga
10861 ccctggagga gcagcagctc cgcgaactgc agcggcacgg ctatgagaac cccacttacc
10921 gcttcctgga ggaacgaccc tgacccggcc cccttcaccc cttcagccga gcccagacct
10981 cccctcttcc tggagcccca gaaccccaac tcccagccta gggcagcagg gagtcttgaa
11041 gtgatcattt cacacccttt tgtgagacgg ctggaaattc ttatttcccc tttccaattc
11101 caaaattcca tccctaagaa ttcccagata gtcccagcag cctccccacg tggcacctcc
11161 tcaccttaat ttatttttta agtttattta tggctcttta aggtgaccgc caccttggtc
11221 ctagtgtcta ttccctggaa ttcaccctct catgtttccc tactaacatc ccaataaagt
11281 cctcttccct accaggcca
```

### Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

The following protocols are provided to facilitate the practice of the present invention.

The following examples are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example I

### Methods

### Synthesis and Preparation of Wild Type and Mutant Human and Rat IAPP Peptides.

IAPP and IAPP analogs were either provided by Prof. Daniel Raleigh at the State University of New York at Stony Brook as previously described (Abedini et al. Org. Lett. 2005 Feb. 17; 7(4):693-6; Abedini et al. Anal Biochem. 2006 Apr. 15; 351(2):181-6, the entire content of each of which is incorporated herein in its entirety), or purchased from the KECK Foundation at Yale University. For details pertaining to synthesis, oxidation, and purification of IAPP peptides, see Abedini et al. Org Lett (2005) 7: 693 and Abedini et al. Anal Biochem (2006) 351:181, the entire contents of which are incorporated herein by reference.

### Solid-Phase Peptide Synthesis by FMOC Chemistry.

Peptides were synthesized on a 0.25 mmol scale using an Applied Biosystems 433A Peptide Synthesizer, using 9-fluornylmethoxycarbonyl (Fmoc) chemistry. Solvents used were A.C.S. grade. Fmoc protected pseudoproline (oxazolidine) dipeptide derivatives were purchased from Novabiochem. All other reagents were purchased from Advanced Chemtech, PE Biosystems, Sigma, and Fisher Scientific. Use of a 5-(4′-Fmoc-aminomethyl-3′,5-dimethoxyphenol) valeric acid (PAL-PEG) resin afforded an amidated C-terminus. Standard Fmoc reaction cycles were used. The first residue attached to the resin, pseudoproline dipeptide derivatives, all β-branched residues, and all residues directly following a β-branched residue were double coupled. Peptides were cleaved from the resin using 90% TFA, 3.33% anisole, 3.33% thioanisole and 3.33% ethanedithiol.

### Peptide Purification.

To increase solubility, the crude peptides were partially dissolved in 20% acetic acid (v/v), frozen in liquid nitrogen and lyophilized. This procedure was repeated several times prior to purification. The dry peptides were then redissolved in 35% acetic acid (v/v) and purified via reversed-phase HPLC, using a Vydac C18 preparative column (10 mm×250 mm). A two-buffer system was used, utilizing HCl as the ion pairing agent. Buffer (A) consisted of H₂O and 0.045% HCl (v/v). Buffer (B) consisted of 80% acetonitrile, 20% H₂O and 0.045% HCl (v/v). Purity was checked by HPLC using a Vydac C18 reversed-phase analytical column (4.6 mm×250 mm). Two solvent systems were used. The first was the same HCl buffer system used for initial peptide purification. The second buffer system utilized TFA as the ion pairing agent; where buffer (A) consisted of H₂O and 0.1% TFA (v/v) and buffer (B) consisted of 90% acetonitrile, 9.9% H₂O and 0.1% TFA (v/v).

### Peptide/Protein Identification.

All peptides and proteins were analyzed by MALDI-TOF Mass Spectrometry using a Bruker MALDI-TOF MS, or by Electrospray Mass Spectrometry using a Micromass Platform LCZ single quadrupole instrument, to confirm their identity. Mass spectra were acquired by averaging scans over the m/z ranges of 500-4000 or 1000-5000.

Oxidation to Form the Cys-2 to Cys-7 disulfide.

Disulfide formation was achieved by air oxidation at pH 8.5. Crude peptide was dissolved at 5.7 mg/mL in 6M GuHCl. This solution was diluted with 6 mL 50 mM Tris (pH 8.5) and air oxidation was allowed to proceed for 24 hours. The reaction was monitored by reversed-phase HPLC. The final GuHCl concentration was 0.86M and the final peptide concentration was 0.81 mg/mL. Similar results were obtained when Tris was omitted and the pH was adjusted to 8.5 after diluting the GuHCl peptide solution into H₂O. Samples of fully reduced crude hAmylin₁₋₃₇ were obtained by adding 9.25 mg DTT to 20 mg crude hAmylin₁₋₃₇ in 30 mL 10% acetic acid (v/v).

Recombinant Murine sRAGE.

Human sRAGE was prepared in a baculovirus expression system in the Schmidt laboratory at NYU School of Medicine using Sf9 cells (Clontech, Palo Alto, Calif.; Invitrogen, Carlsbad, Calif.). Serum-free medium containing sRAGE was subjected to FPLC Mono S for purification (Pharmacia). Purified human sRAGE was dialyzed against DDI H₂O in 0.001% acetic acid (pH 5.0) and lyophilized to a dry powder. The identity of the purified human sRAGE was confirmed by MALDI-TOF mass spectroscopy and by western blotting.

### Thioflavin-T Kinetics Assays.

Thioflavin-T fluorescence was used to monitor the time course of h-IAPP amyloid formation in the presence and absence of sRAGE. To identify which form(s) of h-IAPP interact with RAGE, sRAGE was added to h-IAPP at various time points during the h-IAPP amyloid formation reaction. Aliquots of the reaction mixture were analyzed by adding 100 uL aliquots of the amyloid formation reaction to 96-well plates containing 8 uL of 60 uM thioflavin-T solution at various incubation times after initiation of the amyloid formation reaction. Fluorescence was measured using a Beckman Coulter DTX880 fluorescent plate reader (excitation: 445 nm and emission: 485 nm). Final solution conditions contained 16 mM tris HCl and 65 μM thioflavin-T (pH 7.4). The peptide concentrations for the kinetic assays were 20 μM h-IAPP or rat IAPP, and 20 μM sRAGE. All values represent means±SEM (n=3).

### Far-UV Circular Dichroism Spectroscopy (CD).

CD measurements of IAPP, sRAGE and IAPP/sRAGE reactions were taken at various incubation times. Far-UV CD experiments monitor the development of secondary structure of h-IAPP species and give insight into the conformational requirement for IAPP/RAGE-binding. All CD experiments were performed on an Applied Photophysics circular dichroism spectrophotometer by directly transferring 300 μL of peptide solution from kinetic assays into a 0.1 cm quartz cuvette a few minutes prior to data collection. The CD spectra for sRAGE and tris HCl buffer was subtracted from all RAGE/IAPP mixtures. The final far-UV CD data was collected over the range of 190 to 260 nm. Final solution conditions contained 16 mM tris HCl (pH 7.4). The peptide concentrations for the kinetic assays were 20 μM h-IAPP or rat IAPP, and 20 μM sRAGE.

### Transmission Electron Microscopy (TEM).

Transient kinetic species of h-IAPP were characterized by TEM. TEM images confirm the presence or absence of amyloid which were indicated by thioflavin-T fluorescence. Aliquots (4 μl) were removed from the reaction mixtures monitored by thioflavin-T assays, placed on a carbon-coated 200-mesh copper grid and negatively stained with saturated uranyl acetate. The samples were imaged with a Philips CM12 transmission electron microscope at the New York University electron microscopy core facility. Final solution conditions contained 16 mM tris HCl (pH 7.4). The peptide concentrations for the kinetic assays were 20 μM h-IAPP and 20 μM sRAGE.

### Surface Plasmon Resonance Spectroscopy (SPR)/BIACore.

The ability of different kinetic species to bind RAGE was tested using SPR. sRAGE was immobilized on a C-4 sensor chip. A 20 μM h-IAPP solution was prepared in 16 mM tris HCl buffer (pH 7.4) and aliquots of the reaction were analyzed for binding at various incubation times after peptide dissolution. All SPR binding experiments were carried out on a GE Healthcare SPR instrument in the laboratory of Dr. Donald Landry at Columbia University Medical Center.

### Tryptophan Fluorescence Quenching Assays.

The quenching of tryptophan fluorescence indicates binding. Fluorescence measurements were made at right angle in a 10 cm dual path length quartz cuvette, using a Photon Technology International fluorescent spectrometer (280 nm excitation and 350 nm emission). Background fluorescence from buffer and IAPP peptides were negligible. The fluorescence quantum yield reported for each time point is an average of 20 reads over 20 seconds (2.5 nm bandwidth and 1 second integration time). Final solution conditions contained 16 mM tris HCl (pH 7.4). The peptide concentrations for the kinetic assays were 20 μM h-IAPP or rat IAPP, and M sRAGE.

### ANS Binding Assays.

8-Anilinonaphthalenesulfonic Acid (ANS) is a small hydrophobic dye which is been widely used in protein folding studies. It typically binds to partially structured states which are rich in secondary structure, compact, but which have not yet established the final tertiary structure, thus this dye can be used to detect a molten globule-like character of proteins. Fluorescence spectra of ANS-peptide complexes were measured using a Spex Fluorolog fluorimeter at 25 C (370 nm excitation and 460 nm emission). ANS-peptide complexes were monitored by adding aliquots of human IAPP to a 1 cm cuvette containing 10 μM ANS at various times along the amyloid formation reaction. Final solution conditions contained 16 mM tris HCl and 10 μM ANS (pH 7.4). The peptide concentrations for the kinetic assays were 20 μM h-IAPP or rat IAPP, and 20 μM sRAGE. All values represent means±SEM (n=3).

### Cultured Rat INS-1β-Cells.

Transformed rat insulinoma-1 (INS-1) β-cells (which express RAGE) are a pancreatic beta cell line commonly used for studies of h-IAPP induced toxicity. INS-1 cells were grown in RPMI 1640 supplemented with 10% fetal bovine serum (FBS), 11 mM glucose, 10 mM Hepes, 2 mM L-glutamine, 1 mM sodium pyruvate, 50 μM β-mercaptoethanol, 100 U/ml penicillin, and 100 U/ml streptomycin. Cells were maintained at 37° C./5% CO₂.

### Culture of Aortic Smooth Muscle Cells.

Mouse vascular SMCs were cultured from the aortas of 10-week-old male mice using a modification of the procedure of Tarvo and Barret. See Tarvo et al. Blood Vessels. 1980; 17:110-116. SMCs were cultured following an explant protocol in accordance with institutional guidelines. Cultures were composed of 95% SM-α-actin positivity based on immunostaining.

### AlamarBlue Cell Viability Assays.

Cyto-toxicity was measured by AlamarBlue assay. INS-1 β-cells were seeded at a density of 30,000 cells per well in 96-well plates and cultured for 24 hours prior to stimulation with IAPP. Human and rat IAPP were dissolved and incubated in 16 mM tris HCl (pH 7.4, 25° C.) prior to cell stimulation. AlamarBlue was diluted ten-fold in culture media and cells were incubated for 5 hours at 37° C. Fluorescence (excitation 530; emission 590 nm) was measured on a Beckman Coulter DTX880 fluorescent plate reader. Values calculated were relative to those of control cells treated with buffer only. All values represent means±SEM (n=3).

RNA Isolation and Quantitative Real Time PCR (qRT-PCR).

Assays for qRT-PCR were carried out in 6-well plates. Total cellular RNA was isolated from h-IAPP stimulated β-cells using Qiagen RNA isolation kit, and the quality of RNA samples was determined by measurement of 260:280 ratio. Only samples with a 260:280 ratio of 2.0 or higher were used for reverse transcription. RNA samples were also treated by RNAse-free DNAse to avoid genomic DNA contamination. One microgram of RNA from each sample was reverse-transcribed to cDNA using MCP-1 and IL-1β primers. Non-template negative controls were also performed to monitor non-specific reactions. RNA isolated from rat IAPP and non-h-IAPP treated beta cells were used as negative controls. Equal amounts of RNA were used in each qRT-PCR reaction, and 18S was used as the internal control for amplification at the same time. qRT-PCR reactions were carried out using an Applied Biosystems 7500 RealTime PCR machine. The relative mRNA contents were normalized using 18S and quantification was carried out using qRT-PCR Quantifier Software.

### RAGE-Block Assays.

Beta cells were plated at a density of 30,000 cells per well in 96-well plate the night before the experiment. Cells were pre-treated with either anti-RAGE blocking IgG or control IgG for 2.5 hrs prior to 5 hrs stimulation with 14 uM h-IAPP toxic species or 1:1 mixture of sRAGE/hIAPP (28 micromolar total protein). hIAPP toxic species were produced by incubating a solution of 20 uM IAPP at room temp for 10 hrs. Prevention of formation of hIAPP toxic species was accomplished by reconstituting 20 micromolar dry hIAPP with a solution of 20 micromolar sRAGE.

### Light Microscopy.

Changes in cell morphology were examined by light microscopy to provide a second method of evaluating cell viability. Transformed rat INS-1 beta cells were photographed immediately prior to assessment of toxicity by Alamar blue cell viability assays. Images were taken using an Olympus BX-61 light microscope.

### Pancreatic Islet Isolation.

The pancreas was removed from anesthetized FVB mice and placed into Hanks' balanced salt solution. The pancreas was cut into small pieces, digested with 2.5 mg/mL collagenase (Sigma-Aldrich), and filtered through a 500 μm nylon mesh. The filtrate containing NPI was cultured for 7-10 days at 37° C., 20% CO2 in Ham's F-10 medium supplemented with 10 mM glucose, 50 μM isolbutalmethylxanthine (IBMX; ICN Biomedicals), 0.5% BSA (fraction V, RIA grade, Sigma-Aldrich), 2 mML-glutamine, 3 mMCaCl₂, 10 mMnicotinamide, 100 U/mL penicillin, and 100 g/mL streptomycin (all from Invitrogen). Purified pancreatic islets with intact mantels were hand purified under light microscope and cultured at a density of 25 islets per well in 6-well plates for toxicity assays.

### Immunohistochemistry.

For double insulin and CD45 co-staining, pancreatic sections were blocked in PBS containing 2.0% normal goat serum (Vector Laboratories) and incubated with guinea pig anti-insulin antibody (Dako) at a 1:100 dilution in PBS/1% BSA for one hour, followed by incubation with Texas Red-conjugated goat anti-guinea pig antibody (Jackson ImmunoResearch) for 1 h. All steps were performed at room temperature.

p-Cyanophenylalanine IAPP Kinetics Assays.

All assays were performed on a Photon Technology International fluorescence spectrophotometer. Thioflavin T fluorescence was excited at 450 nm and monitored at 485 nm. p-Cyanophenylalanine fluorescence was excited at 240 nm and the emission was monitored at 296 nm. The emission and excitation slits were set to 5 nm and a 1.0-cm cuvette was used for all experiments. The fluorescence of thioflavin-T and p-Cyanophenylalanine was measured simultaneously for the same sample in dual-dye mode during kinetic runs, which allows kinetic traces to be collected in an interleaved fashion.

### Results

Time Resolved Studies of Amyloid Formation and Cell Toxicity Indicate that IAPP Toxic Species are Soluble, Transient Intermediates:

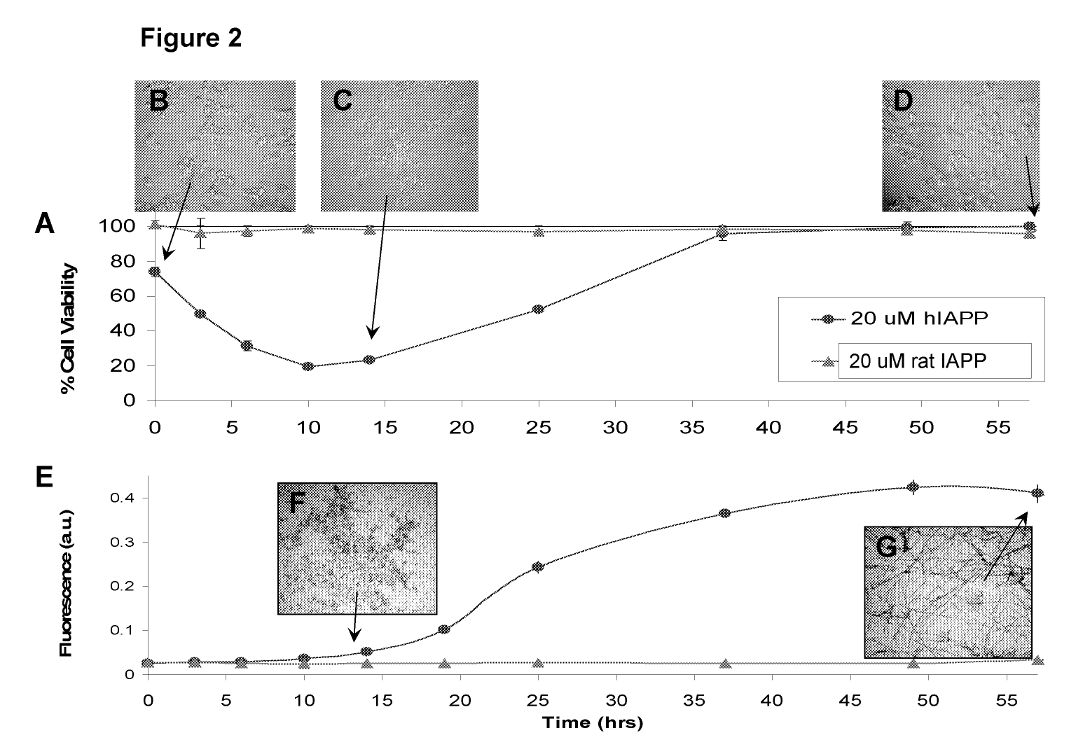

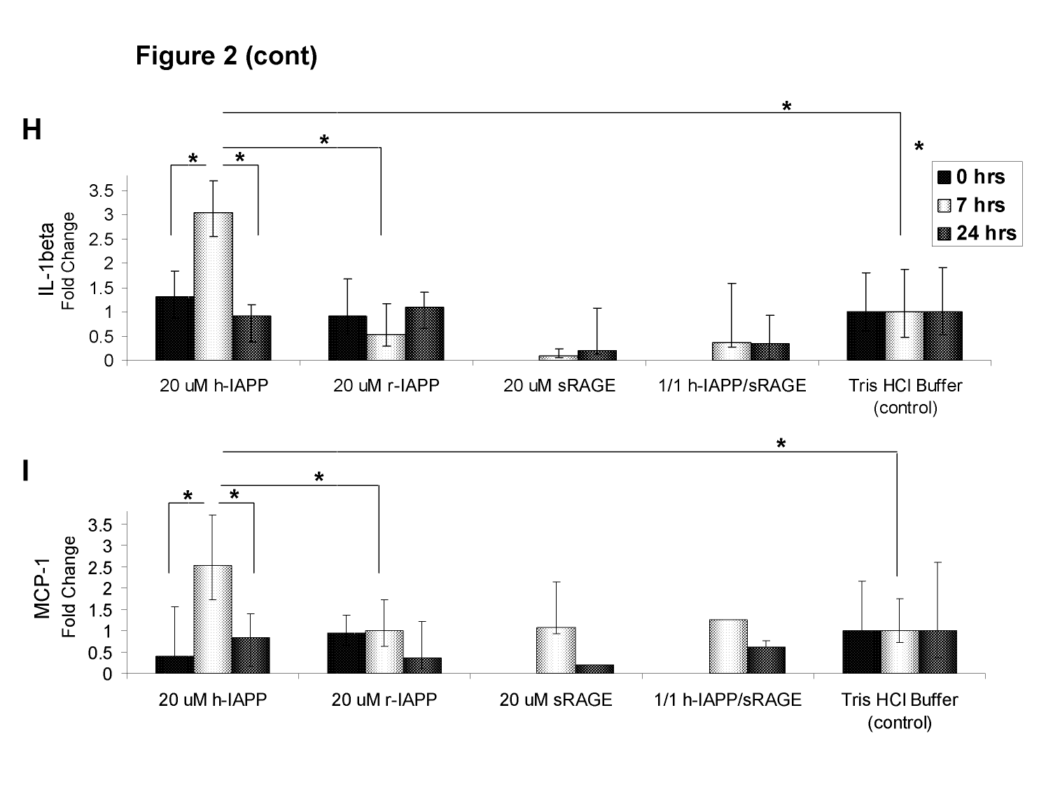

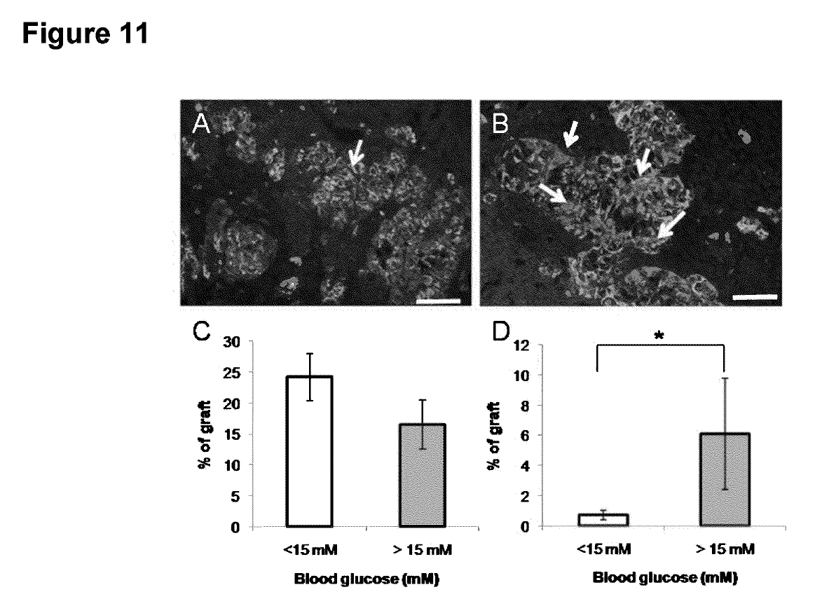

Using a combination of time-dependent biophysical and biological assays, we demonstrate that the cytotoxic species produced during amyloid formation by h-IAPP are transiently populated pre-amyloid intermediates. Human and rat IAPP were incubated in buffer at room temperature for various times and then applied to transformed rat insulinoma-1 (INS-1) β-cells. Cell toxicity was monitored by AlamarBlue cell viability assays and light microscopy (FIGS. 2A-D). These experiments are fundamentally different from the more common protocol in which peptide is applied to cells at time zero and toxicity monitored. The time course of amyloid formation was monitored by thioflavin-T fluorescence and by recording transmission electron microscopy (TEM) images of aliquots removed at different time points (FIGS. 2E-G). Thioflavin-T has been shown to reliably report on amyloid formation by IAPP and does not alter the kinetics of IAPP self assembly. The striking result was the observation of a “wave” of toxicity (FIG. 2-A). No toxicity was observed when h-IAPP amyloid fibrils were added to cultured cells (FIGS. 2-A, D, E and G). In contrast, maximum toxicity was observed at intermediate time points (FIGS. 2-A and C). The peak in toxicity occurs before the observation of amyloid fibrils as judged by thioflavin-T binding assays and TEM (FIGS. 2E and F). Similar results were obtained when h-IAPP lag phase intermediates were added to cultured mouse pancreatic islets and aortic smooth muscle cells (FIGS. 11A and B). The decrease in cell viability is accompanied by an increase in MCP-1 and IL-1β mRNA expression, indicating that IAPP toxic species trigger pro-inflammatory cellular responses (FIGS. 2H and I; FIG. 11C). Rat IAPP does not induce up-regulation of these pro-inflammatory cytokines. No toxicity was observed at any time point when non-amyloidogenic rat IAPP was added to cultured (3-cells.

Changes in The Length of The Lag Phase Induced by Changes in Protein Concentration or Temperature Correlate with Changes in the Duration of Toxicity:

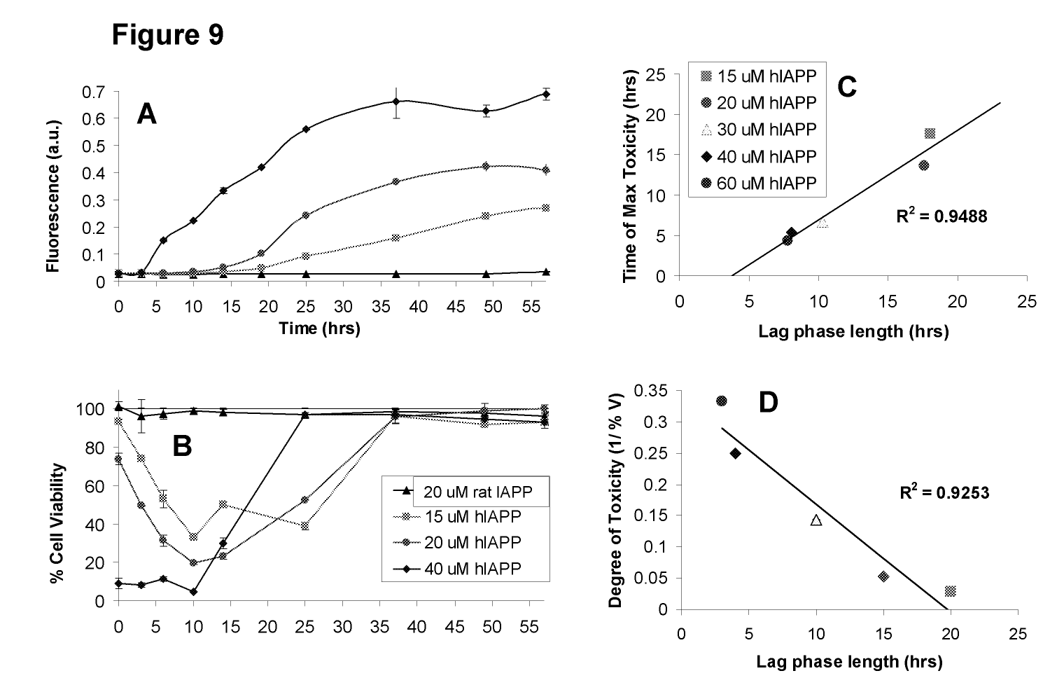

We hypothesized that if toxic intermediates were on pathway to amyloid formation, then there should be a direct correlation between the length of the lag phase and the duration of toxicity. To test this we conducted side-by-side thioflavin-T and AlamarBlue cell viability assays of h-IAPP at different concentrations and temperatures. IAPP aggregation kinetics is concentration-dependent. Decreasing the concentration leads to an increase in the length of the lag phase (FIG. 9A). Likewise, an increase in h-IAPP concentration leads to a shortening of the lag phase and an increase in the rate of aggregation. A decrease in IAPP concentration leads to an increase in the lifetime of the toxic species, where by toxicity has slower on-set and longer duration (FIG. 9B). In contrast, an increase in peptide concentration leads to a shorter lag phase and a shorter duration of toxicity.

The length of the lag phase can also be controlled by altering the temperature at which h-IAPP is incubated. Lower temperatures decrease the rate of amyloid formation and lead to a longer lag phase. h-IAPP was incubated at 15° C.; aliquots were removed at various times and added to cells. The experiment demonstrates that an increase in the lag phase leads to a longer lifetime of the toxic intermediates. A strong linear correlation over a wide range of concentrations is observed if one plots the length of the lag phases versus the duration of toxicity (FIGS. 9C and D), indicating a direct relationship between the kinetics of aggregation (i.e. length of lag phase) and the duration of toxicity.

Altering the Duration of the Lag Phase by the Use of Amyloid Inhibitors or by Mutation Leads To a Correlated Change in the Duration of Toxicity:

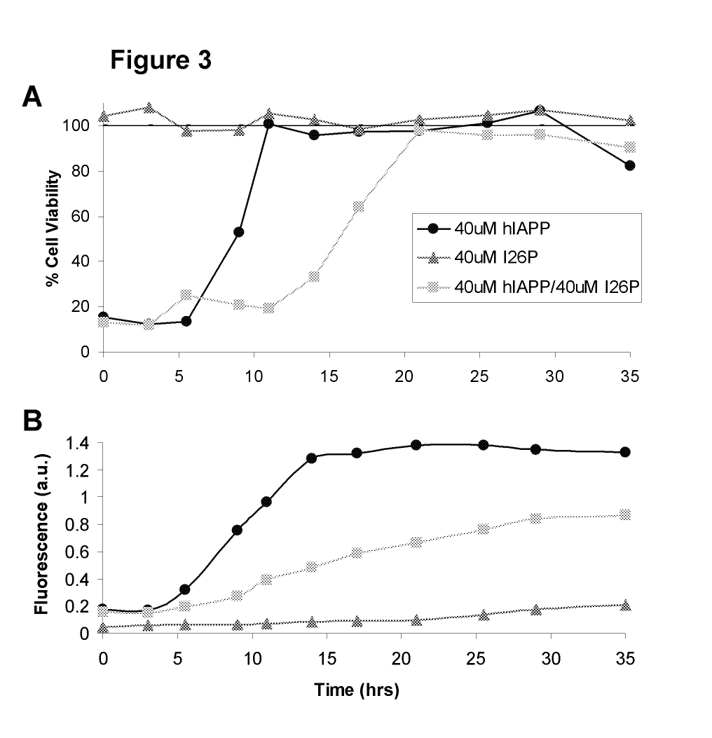

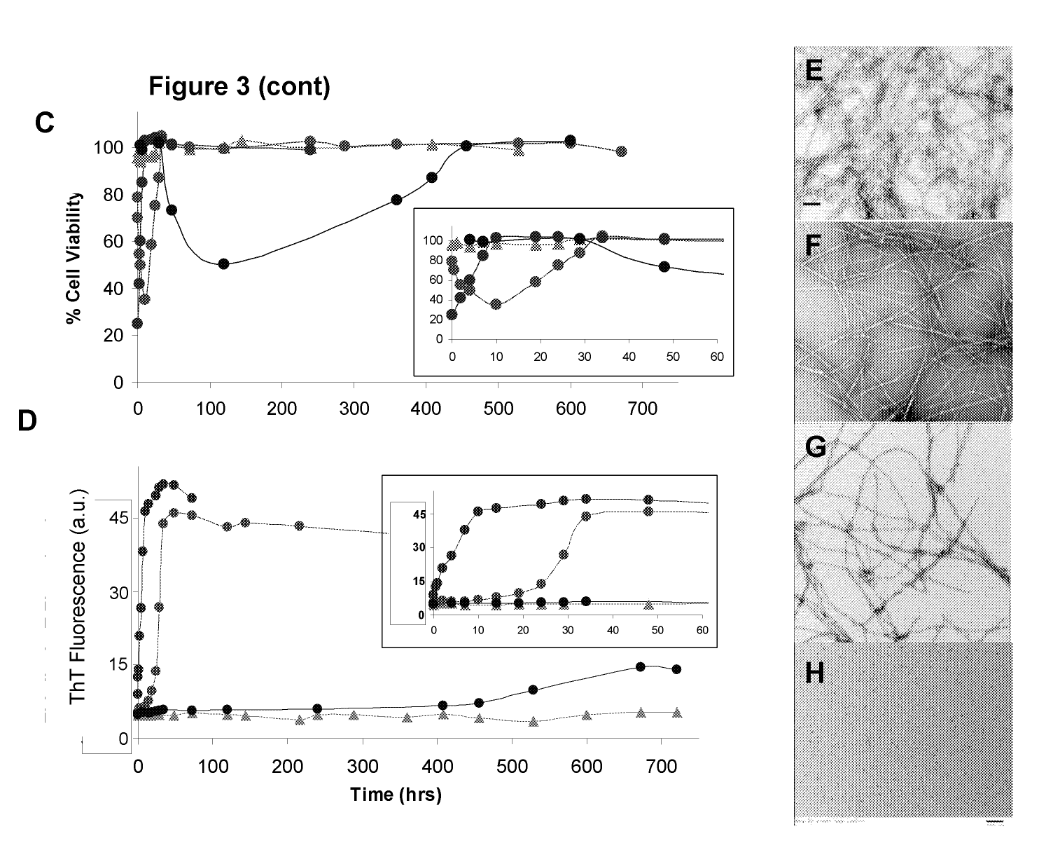

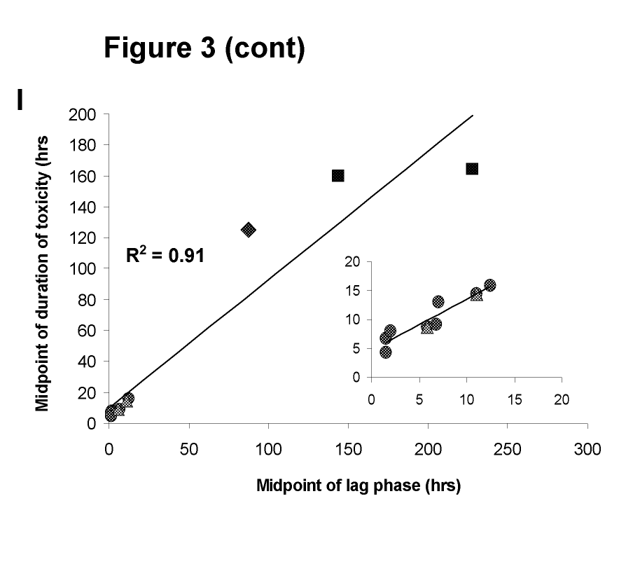

Amyloid inhibitors can be used to probe the nature of the toxic species. Consider an inhibitor that significantly reduces amyloid fibril formation and lengthens the lag phase, but does not prevent the accumulation of wild type amyloid intermediates. If fibrils are toxic, then the inhibitor should reduce toxicity. On the other hand, if intermediates are toxic, then the inhibitor should not reduce toxicity. The I26P point mutation converts wild type h-IAPP into a potent inhibitor of h-IAPP amyloid formation (Fanling et al. J. Am. Chem. Soc. 2010 132(41):14340-2). I26P-IAPP, which does not form amyloid, is not toxic by itself (FIGS. 3A and B). Slowing down the rate of h-IAPP amyloid formation by the addition of the inhibitor increases the duration of toxicity. This result supports our hypothesis that h-IAPP intermediates are toxic, and shows that a good inhibitor of amyloid formation can sometimes be deleterious for cell viability. This indicates that caution must be taken if in vitro biophysical assays are used to develop leads for anti-amyloid agents since drugs that lead to the build up of toxic pre-fibrillar intermediates could be harmful.

A set of Ser-20 mutations provide an additional test of the nature of the toxic species. Ser-20 is located at a critical position in the h-IAPP sequence and modulates its aggregation kinetics. Substitution of Ser-20 with a glycine abolishes the lag phase and increases the rate of amyloid formation (Cao et al. J Mol. Biol. 2011 Dec. 21. [Epub ahead of print]). In contrast, substitution with a lysine significantly decreases the rate of amyloid formation. We carried out side-by-side toxicity and kinetics experiments on the two Ser-20 mutants, wild type h-IAPP (positive control) and rat IAPP (negative control) (FIGS. 3C-H). The faster rate of aggregation of S20G-IAPP leads to a faster onset and shorter duration of toxicity compared to wild type. On the other hand, the slower aggregation rate of S20K-IAPP shifts the onset of toxicity to later time points and increases its duration. The time point of maximum toxicity for wild type h-IAPP, S20G-IAPP and S20K-IAPP shifts with the midpoint of their respective lag phases (T_{ML}). We next examined an h-IAPP triple mutant, 3×L-IAPP, in which leucines replaced three aromatic residues [See FIG. 1B, phenylalanine (F) at amino acid positions 15 and 23; and tyrosine (Y) at amino acid position 37]. This mutant forms amyloid 6-fold slower than wild type. The triple mutant leads to a longer duration of toxicity than observed with wild type, and provides additional evidence that the lag phase species are the toxic entities.

Plotting the length of the lag phases for all of the variants of human and IAPP versus their respective duration of toxicity reveals a strong linear correlation, demonstrating a direct relationship between the kinetics of aggregation and the duration of toxicity (FIG. 31, and Table 2).

IAPP Toxic Species are Loosely Packed Soluble Oligomers which Lack Significant Beta-Sheet Structure:

The ability to monitor toxicity in a time-resolved fashion allows us to characterize the toxic species under well defined conditions. Characterization of h-IAPP by far ultra violet circular dichroism (CD) at time points of toxicity show the development of some partial helical structure, but no beta sheet structure (FIG. 4A). Infrared spectroscopy (IR) is complementary to CD and is particularly suited for the detection of beta sheet structures. Two-dimensional IR (2D-IR) has recently been applied to amyloid systems and has been shown to be a sensitive probe of beta sheet structure (Middleton et al. Nat. Chem. 2012 Mar. 11; 4(5):355-60).

We recorded 2D-IR spectra of the intermediate species. The results are consistent with a lack of beta sheet structure and support the CD data (FIG. 4B). Ultracentrifugation studies show that the toxic species are soluble (FIG. 4C). Samples of h-IAPP intermediates and h-IAPP amyloid fibrils were pelleted at 20,000 G for 20 minutes and the amount of soluble peptide remaining in the supernatant was measured. Fully 6.5% of the protein was pelleted in the amyloid sample and 93.5% remained in solution. Characterization of the species in the supernatant and resuspended pellet by thioflavin-T and TEM, before addition of the solutions to cultured cells, confirm the absence of amyloid in the soluble phase and the presence of amyloid in the resuspended pellet. CD spectra taken of the soluble phase species shows no change in secondary structure after ultracentifugation (FIG. 4C). The cell viability results reveal that soluble h-IAPP in solution is toxic to beta cells, while the resuspended pellet is not. ANS binding studies show that the h-IAPP intermediate is not a molten globule (FIG. 4D). ANS is a small hydrophobic dye which has been widely used in protein folding studies. It typically binds to partially structured states which are rich in secondary structure, compact, but which have not yet established the final tertiary structure. No ANS binding was observed to the monomeric form of h-IAPP or to any of the intermediates. The dye did bind to the final amyloid fibrils; an effect that is mediated in part by electrostatic interactions. A set of p-cyano-phenylalanine (p-cyanoPhe) analogs of h-IAPP were examined to probe further the nature of the toxic intermediate species. h-IAPP contains two phenylalanines at positions 15 and 23 and a single tyrosine located at the C-terminus. See FIG. 1B. A set of three analogs were prepared in which one aromatic residue was replaced by p-cyanoPhe. The substitutions do not perturb amyloid formation relative to wild type IAPP (Marek et al. Chembiochem. 2008 Jun. 16; 9(9):1372-4). p-CyanoPhe fluorescence is high when the cyano group is hydrogen bonded and low when it is not. It can also be quenched via FRET to tyrosine. p-CyanoPhe fluorescence is high for unaggregated IAPP and is quenched in the amyloid fibers. The intermediate species have high p-cyanoPhe fluorescence intensity and the value is the same as that of freshly dissolved material. These experiments show that F15, F23 and Y37 are solvent exposed in the intermediate and rule out conformations in which the C-terminal Tyr is close to either F15 or F23.

Transiently Populated, Pre-Fibrillar h-IAPP Intermediates are Ligands of RAGE:

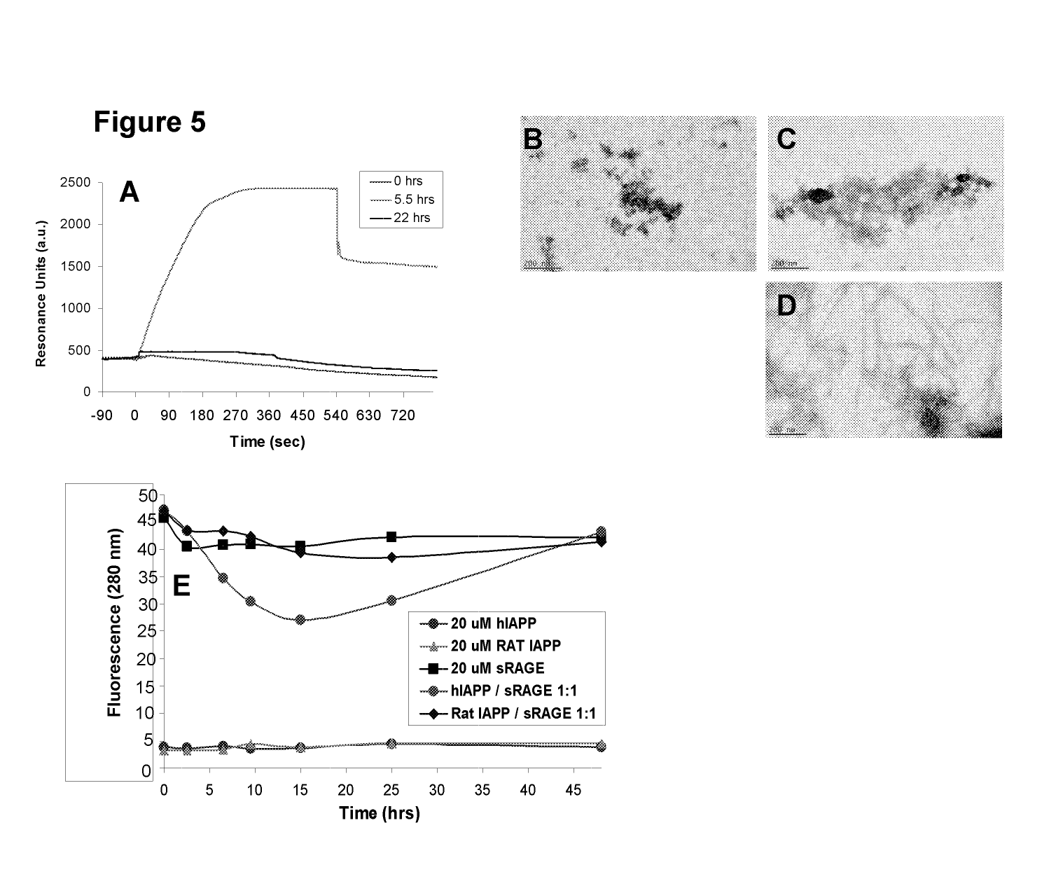

Using surface plasmon resonance (SPR), we demonstrate that RAGE binds transiently populated intermediates, but does not bind to h-IAPP monomers or amyloid fibrils (FIG. 5A). SPR studies were accompanied by TEM experiments to confirm the presence or absence of amyloid fibrils (FIGS. 5B-D). The SPR results are supported by tryptophan fluorescence quenching studies of RAGE-IAPP binding (FIG. 5E). sRAGE has a large hydrophobic patch containing three solvent exposed tryptophans near the C′D loop of the V-type immunogloblulin-like domain (FIG. 5-F) [Park et al. (2010) JBC 285(52), 40762; Koch et al. (2010) Structure 13; 18(10), 1342]. Binding of ligands to this region should alter the environment of the tryptophans and lead to a change in their fluorescent quantum yield. Thus, the quenching of tryptophan fluorescence can be used to monitor binding. We added sRAGE to h-IAPP at a 1:1 molar ratio at different time points during the course of the amyloid formation reaction and observed a wave of fluorescence quenching, whose time course mirrors that of the wave of toxicity under the same experimental conditions, arguing that h-IAPP toxic intermediates and RAGE-binding intermediates are the same. Similar results were obtained upon addition of sRAGE to h-IAPP at a 1:2 molar ratio. The data suggests that the V-type domain of RAGE is involved in RAGE-IAPP interactions. No binding of sRAGE to the non-toxic rat IAPP was observed at any time point (FIG. 5E).

To test whether the conformational requirement for h-IAPP toxicity is the same as for h-IAPP-RAGE binding, we tested the ability of the nontoxic, aggregation-prone I26P-IAPP variant of h-IAPP to bind to sRAGE. In this experiment sRAGE was added to I26P-IAPP at different time points over the course of the aggregation reaction and binding was monitored by tryptophan fluorescence quenching. No binding was detected. Difference CD spectra collected at the end point of the reactions provide further evidence that sRAGE does not interact with either I26P-IAPP or rat IAPP. CD spectra of the control peptides by themselves show the expected random coil conformation for I26P-IAPP and rat IAPP. No conformational change was observed when sRAGE was added to either peptide at any time point. This result indicates that sRAGE does not induce amyloid formation by rat or I26P-IAPP.

sRAGE is an Inhibitor of Human IAPP Toxicity:

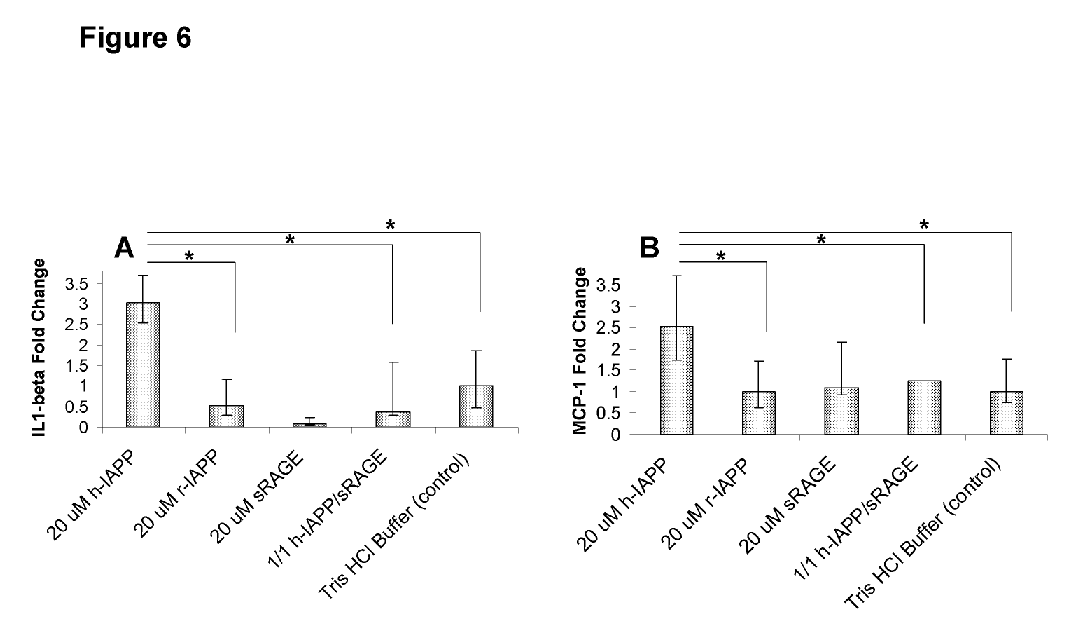

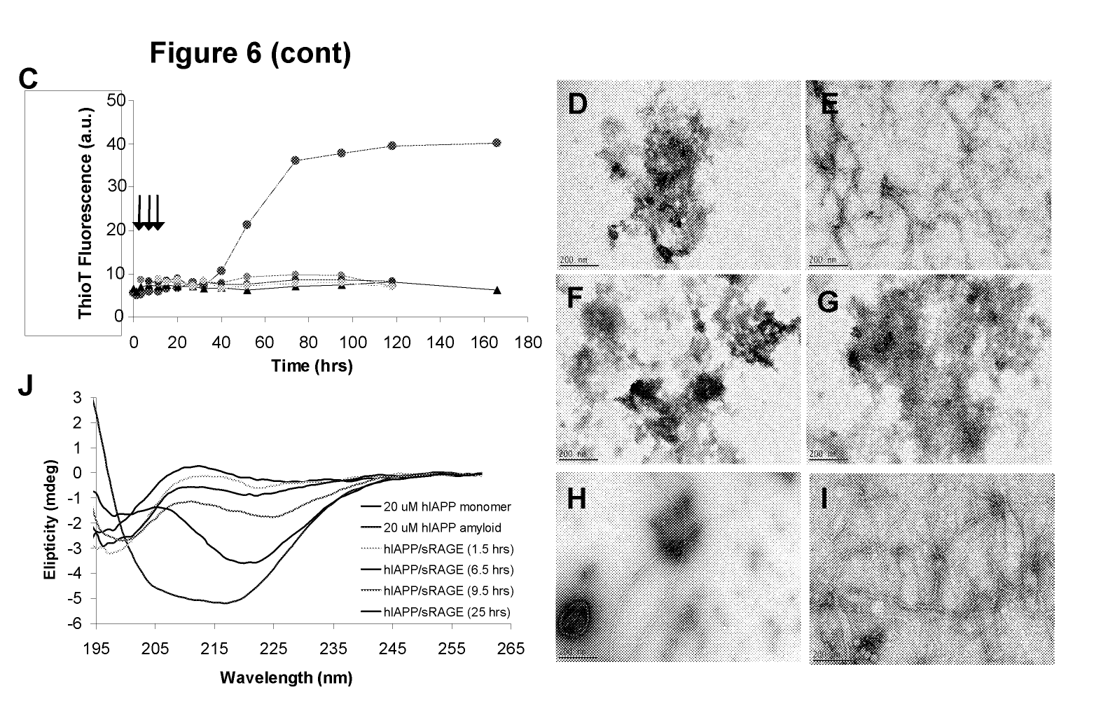

Free sRAGE can act as a dominant negative inhibitor of ligand binding to membrane-associated RAGE. We hypothesized that if h-IAPP binding to RAGE is important for toxicity, then sRAGE should be an inhibitor of h-IAPP toxicity. h-IAPP and rat IAPP were reconstituted with either reaction buffer (control) or a solution of equimolar sRAGE. After 5 hrs of incubation, the absence of amyloid fibrils was confirmed in all conditions by thioflavin-T binding assays, before adding to cultured beta cells. Addition of sRAGE to h-IAPP at a 1:1 molar ratio before time points of toxic species formation protects transformed rat INS-1 β-cells from toxicity. qRT-PCR studies carried out under identical conditions as the toxicity assays show that sRAGE prevents upregulation of MCP-1 and IL-1β mRNA expression induced by h-IAPP intermediates (FIGS. 6A and B). sRAGE is not toxic by itself and does not induce up-regulation of these pro-inflammatory biomarkers.

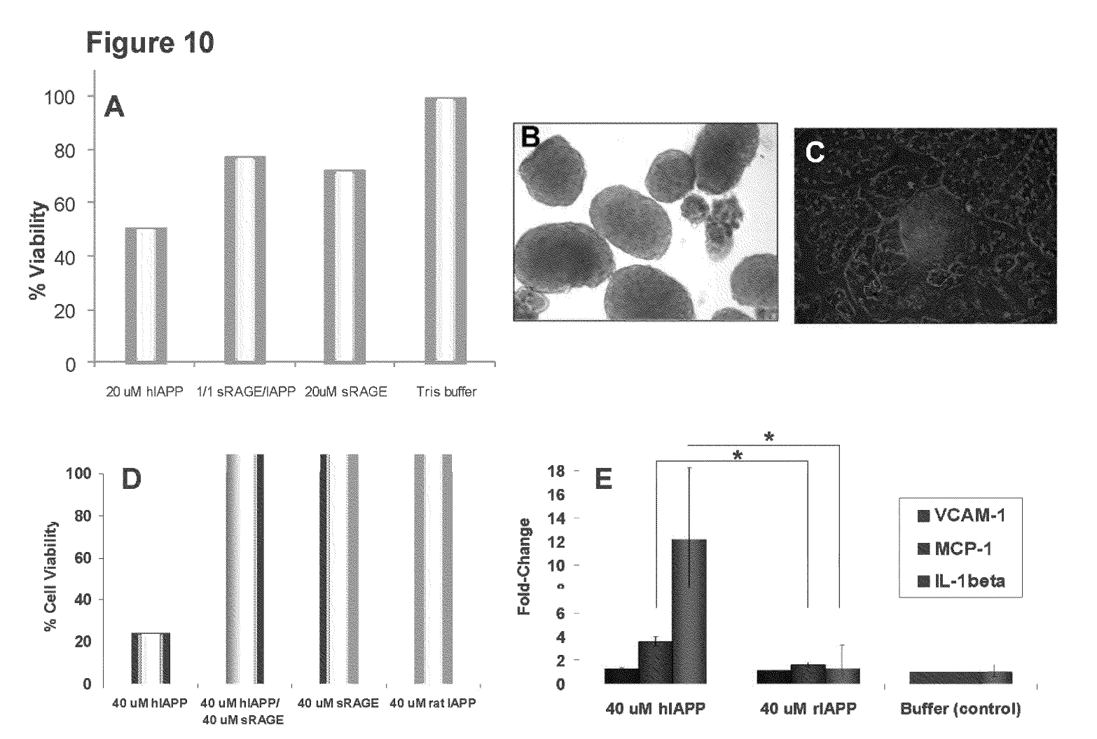

Similar results were obtained with mouse pancreatic islets (FIG. 10A-C). Mouse pancreatic islets were isolated from wild type FVB mice and hand selected for toxicity assays under light microscope. Immunohistochemistry of pancreas sections taken from mice that were the same age, strain and metabolic condition as those used for islet isolation indicated that the islets were healthy and insulin-positive after harvest. Addition of h-IAPP intermediates to cultured islets resulted in 50% loss in viability relative to buffer treatment alone. Addition of sRAGE to h-IAPP at a 1:1 molar ratio inhibited toxicity and restored viability to 80% relative to buffer control, and to 100% relative to sRAGE by itself (FIG. 10A).

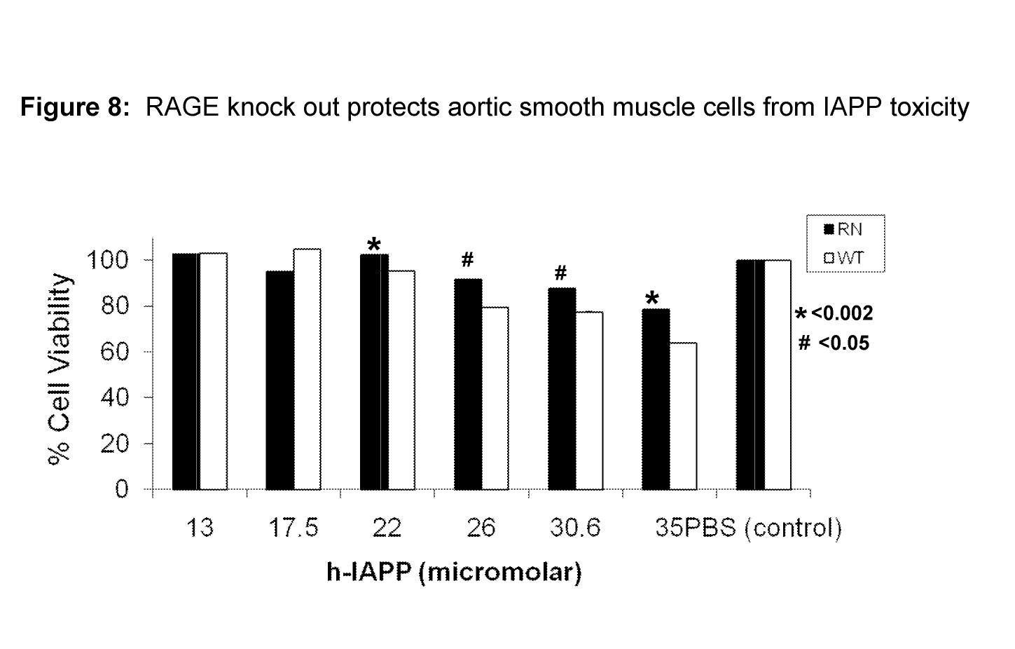

h-IAPP intermediates are toxic to other cells that express RAGE demonstrating that h-IAPP cytotoxicity is not cell-specific to beta cells. AlamarBlue cell viability assays and quantitative real time PCR of mouse aortic smooth muscle cells show that h-IAPP intermediates are toxic to smooth muscle cells (FIG. 8). The decrease in cell viability induced by h-IAPP is accompanied by an increase in MCP-1 and IL-1β mRNA expression, similar to that observed for beta cells. Addition of sRAGE to h-IAPP before time point of toxic species formation blocks smooth muscle cell toxicity.

sRAGE is an Inhibitor of h-IAPP amyloid Formation:

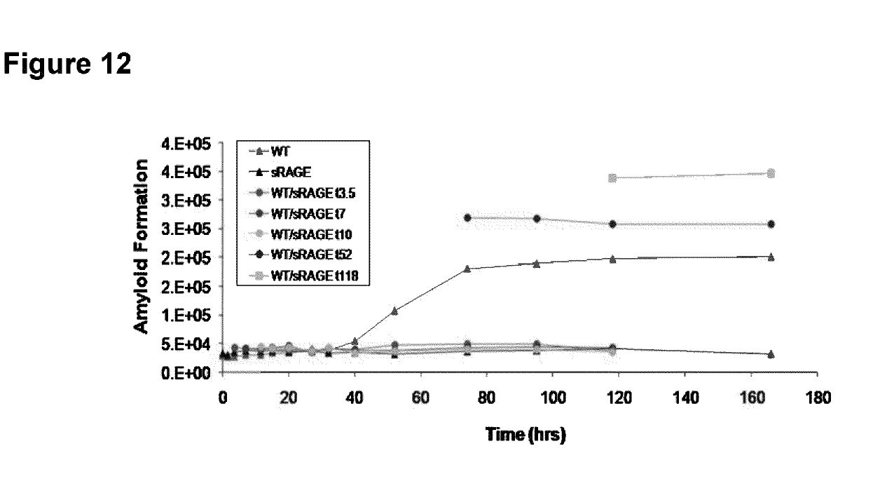

We hypothesized that sRAGE is an inhibitor of h-IAPP amyloid formation, as it binds to toxic intermediates. sRAGE was added to h-IAPP (1:1 molar ratio) at 0, 1.5, 6.5, 9.5, 15 and 25 hours after h-IAPP amyloid formation was initiated; thioflavin-T assays and TEM were used to characterize the species at each time point (FIG. 6). The results show that addition of sRAGE before the midpoint of the h-IAPP amyloid formation reaction inhibits amyloid formation (FIG. 6). TEM images show that addition of sRAGE to h-IAPP before or at very early time points of toxic species formation leads to complete inhibition of h-IAPP amyloid formation, even after two weeks of additional incubation. This result demonstrates an extended effect on amyloid formation and suggests a stable interaction between sRAGE and h-IAPP. The TEM and thioflavin-T results were confirmed by using difference CD spectroscopy to probe the development of secondary structure as a function of time (FIG. 6). Addition of sRAGE before time points of h-IAPP toxic species formation leads to inhibition of β-sheet formation by h-IAPP (FIG. 6). The effect of sRAGE on h-IAPP secondary structure development is much less dramatic if it is added to h-IAPP at later time points and no effect is observed when sRAGE is added to amyloid fibrils. Similar results were obtained upon addition of sRAGE to h-IAPP at a 1:2 molar ratio. See FIG. 12.

These biophysical experiments indicate at which time point human IAPP becomes competent to bind sRAGE and further confirm that transiently populated intermediates of h-IAPP are ligands of RAGE. Addition of sRAGE to h-IAPP at later time points of toxicity leads to significant reductions in h-IAPP beta sheet structure and amyloid formation (FIG. 6). The effect of sRAGE on h-IAPP amyloid formation decreases in a time-dependent manner, as sRAGE is added to h-IAPP at later time points. In contrast, no effect on h-IAPP amyloid formation is seen when sRAGE is added after time points of toxic species formation (FIG. 6). The results show that h-IAPP intermediates are ligands of RAGE and demonstrate that sRAGE is an effective inhibitor of h-IAPP toxicity and amyloid formation.

Genetic Deletion of RAGE or Blocking Rage-IAPP Interactions Protects Cells from Toxicity, Supporting a RAGE-Mediated Mechanism of IAPP-Induced Cellular Toxicity:

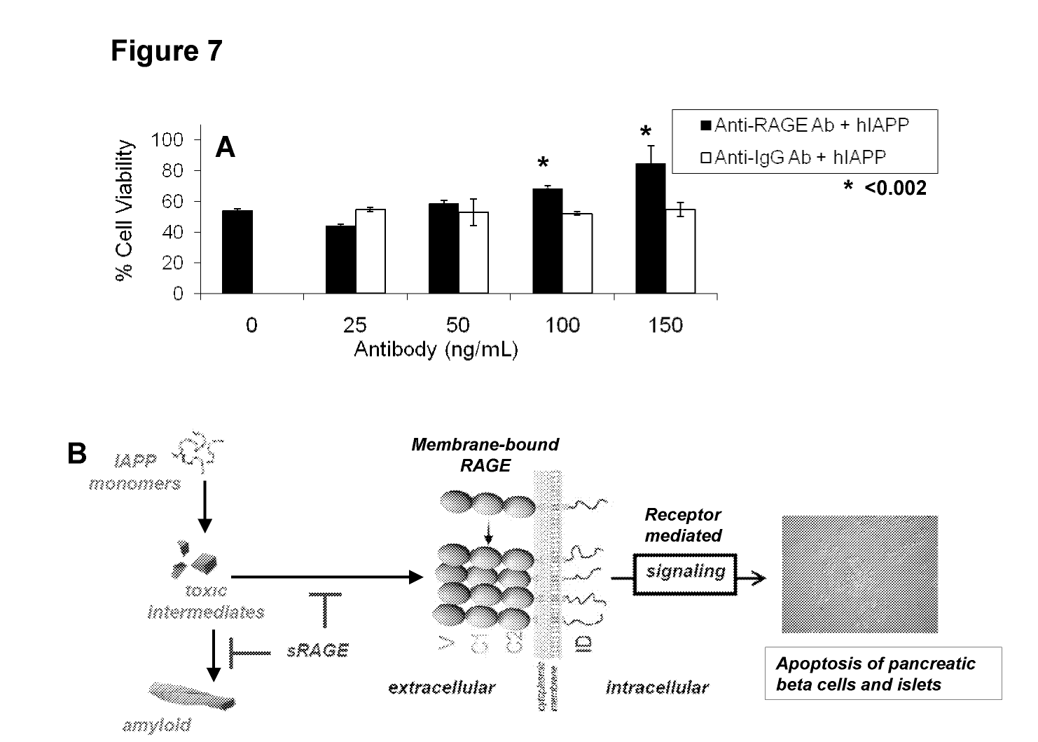

If RAGE plays an important role in IAPP-induced beta cell toxicity, then blocking RAGE with anti-RAGE IgG should protect beta cells at least in part, from h-IAPP toxicity. This is what we observe. We pre-treated beta cells with increasing doses of either an anti-RAGE IgG or a control IgG, followed by stimulation with toxic h-IAPP intermediates. The results show that the anti-RAGE IgG protects beta cells, in part, from h-IAPP toxicity in a dose-dependent manner. No significant change in beta cell toxicity was observed with pre-treatment of increasing concentrations of control IgG (FIG. 7A). If the interaction between sRAGE and h-IAPP is weak, then the anti-RAGE IgG would be expected to compete with sRAGE-bound h-IAPP and displace it. The protective effects of sRAGE are not affected by the presence of anti-RAGE IgG, suggesting a significant and specific interaction between RAGE and h-IAPP toxic intermediates.

We also tested the effect of toxic h-IAPP intermediates on RAGE-null (RN) and wild type (WT) mouse aortic smooth muscle cells (SMCs). SMCs were stimulated with increasing amounts of h-IAPP toxic species. The results show that genetic deletion of RAGE protects SMCs from h-IAPP toxicity compared to WT SMCs (FIG. 8). These findings support our hypothesis that h-IAPP toxic intermediates are ligands of RAGE, and are consistent with a role for RAGE in h-IAPP-induced toxicity in T2D.

FIG. 11 shows the progression of amyloid formation mediated toxicity with respect to the histology of insulin-producing pancreatic β-cells. As depicted therein, rapid amyloid formation is associated with human islet graft failure. Typically, normal histology and morphology of islet cell mass is observed in healthy individuals, which progresses to islet hyperplasia in pre-diabetic states (characterized by insulin resistance), and eventually leads to loss of beta cell mass in diabetes (hyperglycemia/hyperinsulinemia).

### DISCUSSION

We demonstrate herein that transient, pre-fibrillar oligomers that form early in the h-IAPP amyloid formation process are toxic to rat INS-1 pancreatic beta cells, mouse pancreatic islets and mouse aortic smooth muscle cells. We further show that IAPP-induced reduction in cell viability is accompanied by up-regulation of the pro-inflammatory cytokines, MCP-1 and IL-β. Experiments which alter the time course of amyloid formation reveal that the lifetime of the toxic species strongly correlate with the length of the lag phase. Biophysical characterization of the toxic intermediates, moreover, indicates that these species are soluble, loosely packed, are not molten globules, and lack significant β-sheet structure.

The toxic intermediates of h-IAPP are ligands of RAGE and sRAGE effectively inhibits both IAPP toxicity and amyloid formation. These results are consistent with a RAGE-mediated mechanism of IAPP toxicity in T2D. RAGE is a multi-ligand receptor that is expressed in amyloid-rich environments, and is up-regulated in inflammatory disorders such as diabetes. RAGE activates signaling cascades involved in cellular stress responses, including pro-inflammatory cytokine production and apoptosis. Neurotoxic amyloid-β (Aβ) peptides bind to RAGE, and RAGE activation in the brain of individuals with AD has been shown to lead to neurological dysfunction (Yan et al. Restor Neurol Neurosci. 1998 June; 12(2-3): 167-73). sRAGE has the ability to bind toxic intermediates and is also able to inhibit amyloid formation. Molecules with these properties are envisioned as broad therapeutic agents since toxicity in some amyloidoses are mediated by intermediates (FIG. 7B). This work has implications for the treatment of islet Amyloidosis in T2D and may impact the treatment of other amyloidosis diseases, as common structures and mechanisms of toxicity have been proposed for pathological amyloidogenic species derived from different peptides, polypeptides and proteins despite considerable variation in their primary sequences.

### REFERENCES

Chiti, F., and Dobson, C. M. Protein misfolding, functional amyloid, and human disease, Annu Rev Biochem (2006) 75, 333-366.
Sipe, J. D. Amyloidosis (1994) Crit. Rev Clin Lab Sci 31, 325-354.
Selkoe, D. J. Cell biology of protein misfolding: The examples of Alzheimer's and Parkinson's diseases (2004) Nature Cell Biol 6, 1054-1061.
Jahn, T. R., and Radford, S. E. Folding versus aggregation: Polypeptide conformations on competing pathways. Arch Biochem Biophys (2008) 469, 100-117.
Kahn, S. E., Andrikopoulos, S., Verchere, C. B. Islet amyloid: a long recognized but underappreciated pathological feature of type II diabetes (1999) Diabetes, 48, 241-246.
Hull, R. L., Westermark, G. T., Westermark, P., Kahn, S. E. Islet amyloid: a critical entity in the pathogenesis of type 2 diabetes (2004) J. Clin. Endocrin. Metab. 89, 3629-3643.
Potter K J, Abedini A, Marek P, Klimek A M, Butterworth S, Driscoll M, Baker R, Nilsson M R, Warnock G L, Oberholzer J, Bertera S, Trucco M, Korbutt G S, Fraser P E, Raleigh D P, Verchere C B. Islet amyloid deposition limits the viability of human islet grafts but not porcine islet grafts. (2010) PNAS 107, 4305.
Janson, J., Ashley, R. H., Harrison, D., McIntyre, S., Butler, P. C. The mechanism of islet amyloid polypeptide toxicity is membrane disruption by intermediate-sized toxic amyloid particles (1999) Diabetes 48, 491-498
Engel, M. F. Membrane permeabilization by Islet Amyloid Polypeptide. (2009) Chem Phys Lipids 160, 1-10.
Haataja, L., Gurlo, T., Huang, C. J., Butler, P. C. Islet amyloid in type 2 diabetes and the toxic oligomer hypothesis (2008) Endocr Rev 29 303-316.
Zhang, S., Liu, H., Yu, H., Cooper, G. J. Fas-associated death receptor signaling evoked by human amylin in islet beta-cells (2008) Diabetes 57, 348-356.
Yan, S. D., Chen, X., Chen, M., Zhu, H., Roher, A., Slattery, T., Zhao, L., Nagashima, M., Morser, J., Migheli, A., Nawroth, P., Stern, D. M., Schmidt, A. M. RAGE and amyloid-beta peptide neurotoxicity in Alzheimer's disease (1996) Nature 382, 685-691.
Hudson, B. I., Carter, A. M., Harja, E., Kalea, A. Z., Arriero, M., Yang, H., Grant, P. J., Schmidt, A. M. Identification, classification, and expression of RAGE gene splice variants (2008) FASEB J 22, 1572-1580.
Yan, S., Ramasamy, R., Schmidt, A. Receptor for AGE (RAGE) and its ligands-cast into leading roles in diabetes and the inflammatory response (2009) Journal of Molecular Medicine 87, 235-247.
Yan, S. F., Ramasamy, R., Schmidt, A. M. Mechanisms of Disease: advanced glycation end-products and their receptor in inflammation and diabetes complications (2008) Nat Clin Pract Endocrinol Metab 4(5), 285-293.
Clynes, R., Moser, B., Yan, S., Ramasam, R., Herold, K., Schmidt, A. Receptor for AGE (RAGE): weaving tangled webs within the inflammatory response (2007) Current Molecular Medicine 7, 743-751.
Herold, K., Moser, B., Chen, Y., Zeng, S., Yan, S. F., Ramasamy, R., Emond, J., Clynes, R., Schmidt, A, M. Receptor for advanced glycation end products (RAGE) in a dash to the rescue: inflammatory signals gone awry in the primal response to stress (2007) J Leukoc Biol 82, 204-212.
Xie, J., Reverdatto, S., Frolov, A., Hoffmann, R., Burz, D. S., Shekhtman, A. Structural basis for pattern recognition by the receptor for advanced glycation end products (RAGE) (2008) J Biol Chem 283, 27255-27269.
Park, H., Boyington, J. C. The 1.5 Å crystal structure of human receptor for advanced glycation endproducts (RAGE) ectodomains reveals unique features determining ligand binding. (2010) JBC 285(52), 40762.
Koch, M., Chitayat, S., Dattilo, B. M., Schiefner, A., Diez, J., Chazin, W. J., Fritz, G. Structural basis for ligand recognition and activation of RAGE. (2010) Structure 13; 18(10), 1342.
Bierhaus, A., Humpert, P., Morcos, M., Wendt, T., Chavakis, T., Arnold, B., Stern, D., Nawroth, P. Understanding RAGE, the receptor for advanced glycation end products (2005) Journal of Molecular Medicine 83, 876-886.
Schmidt, A. M., Vianna, M., Gerlach, M., Brett, J., Ryan, J., Kao, J., Esposito, C., Hegarty, H., Hurley, W., Clauss, M. Isolation and characterization of two binding proteins for advanced glycosylation end products from bovine lung which are present on the endothelial cell surface (1992) J Biol Chem 267, 14987-14997.
Schmidt, A. Yan, S., Yan, S., Stern, D. The multiligand receptor RAGE as a progression factor amplifying immune and inflammatory responses (2001). Journal of Clinical Investigation 108, 949-955.
Liliensiek, B., Weigand, M., Bierhaus, A., Nicklas, W., Kasper, M., Hofer, S., Plachky, J., Grone, H., Kurschus, F., Schmidt, A. Receptor for advanced glycation end products (RAGE) regulates sepsis but not the adaptive immune response (2004) J Clin Invest 113, 1641-1650.
Yan, S. D., Zhu, H., Zhu, A., Golabek, A., Roher, A., Yu, C., Soto, C., Schmidt, A. M., Stern, D. M.; Kindy, M. Receptor glycation dependent cell stress and amyloid accumulation in systemic amyloidosis (2000) Nat. Med. 6, 643-651.
Hori, O., Brett, J., Slattery, T., Cao, R., Zhang, J., Chen, J., Nagashima, M., Lundh, E., Vijay, S., Nitecki, D. The receptor for advanced glycation end products (RAGE) is a cellular binding site for amphoterin. Mediation of neurite outgrowth and co-expression of rage and amphoterin in the developing nervous system (1995) J Biol Chem 270, 25752-25761.
Sturchler, E., Galichet, A., Weibel, M., Leclerc, E., Heizmann, C. W. Site-specific blockade of RAGE-Vd prevents amyloid-beta oligomer neurotoxicity (2008) J Neurosci. 28(20), 5149-58.
Ferrone, F. Nucleation: The connections between equilibrium and kinetic behavior. Method. Enzymol. 2006; 412: 285-299.
Wetzel, R. Kinetics and thermodynamics of amyloid fibril assembly. Acc. Chem. Res. 2006; 39 (9): 671-679.
Harper, J D, Lansbury, P T. Models of amyloid seeding in Alzheimer's and scrapie: Mechanistic truths and physiological consequences of the timedependent solubility of amyloid proteins. Annual Rev. Biochem. 1997; 66: 385-407.
Oosawa, F, Asakura, S. Thermodynamics of the polymerization of protein. 1975. Academic Press, New York, N.Y.
Abedini A, Raleigh D P. Incorporation of pseudoproline derivatives allows the facile synthesis of human IAPP, a highly amyloidogenic and aggregation-prone polypeptide. Org. Lett. 2005 Feb. 17; 7(4):693-6.
Abedini A, Singh G, Raleigh D P. Recovery and purification of highly aggregation-prone disulfide-containing peptides: application to islet amyloid polypeptide. Anal Biochem. 2006 Apr. 15; 351(2):181-6.
Andisheh Abedini and Daniel P. Raleigh. Protein Misfolding: Amyloid Formation. (2008) Wiley Encyclopedia of Chemical Biology. John Wiley and Sons, Inc.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for screening to identify an inhibitor of amyloidogenic polypeptide self-aggregation into amyloids, the method comprising the steps of (a) providing an amyloidogenic polypeptide under conditions that permit self-assembly and adding a candidate agent thereto, wherein the candidate agent is added to the polypeptide during lag phase of amyloid formation of the polypeptide, wherein oligomeric precursors of mature amyloid fibrils are formed and (b) detecting the degree of oligomerization of the amyloidogenic polypeptide at equilibrium in the presence of the candidate agent and comparing that to the degree of oligomerization of the amyloidogenic polypeptide at equilibrium in the absence of the candidate agent, wherein a reduction in the degree of oligomerization of the amyloidogenic polypeptide at equilibrium in the presence of the candidate agent relative to that detected in the absence of the candidate agent indicates that the candidate agent is an inhibitor of amyloidogenic polypeptide self-aggregation into amyloids.

2. (canceled)

3. The method of claim 1, wherein the amyloidogenic polypeptide is set forth in Table 1.

4. The method of claim 3, wherein the amyloidogenic polypeptide is human islet amyloid polypeptide (IAPP), amyloid-β (Aβ), or α-synuclein.

5. The method of claim 1, wherein the lag phase of amyloid formation is between about 0-500 hours after the amyloidogenic polypeptide is provided under conditions that permit self-assembly.

6. The method of claim 1, wherein equilibrium is reached after 40-1000 hours after dissolution of the amyloidogenic polypeptide as provided under conditions that permit self-assembly.

7. The method of claim 1, wherein the candidate agent is added at the onset of the assay or before the midpoint of the lag phase of amyloid formation.

8. The method of claim 1, wherein the amyloidogenic polypeptide is labeled with a detectable label or the candidate agent is labeled with a detectable label.

9. The method of claim 1, wherein the amyloidogenic polypeptide is labeled with a first detectable label and the candidate agent is labeled with a second detectable label.

10. The method of claim 1, wherein the amyloidogenic polypeptide is labeled with a first detectable label and the candidate agent is labeled with a second detectable label and detectable signal of the first and/or second detectable label is altered when the first and second labels are in close proximity.

11. The method of claim 1, further comprising measuring cellular toxicity of the amyloid precursors in the presence of the candidate agent and the absence of the candidate agent, wherein a reduction in toxicity in the presence of the candidate agent indicates that the oligomeric precursors are toxic intermediates and the candidate agent is an inhibitor of cellular toxicity mediated by the toxic intermediates.

12-16. (canceled)

17. A method of treating a subject afflicted with an amyloidoses, the method comprising administering to the subject a therapeutically effective amount of the inhibitor of amyloidogenic polypeptide self-aggregation into amyloids of claim 1, wherein the administering reduces amyloidogenic polypeptide self-aggregation, thereby treating the subject afflicted with an amyloidoses.

18. The method of claim 17, wherein the amyloidoses is diabetes, Alzheimer's Disease (AD), or Parkinson's Disease (PD).

19. The method of claim 17, wherein the inhibitor of amyloidogenic polypeptide self-aggregation into amyloids of claim 1 is sRAGE or a functional fragment thereof or an anti-RAGE antibody that inhibits binding of the amyloidogenic polypeptide to RAGE.

20. A method for reducing islet transplant failure in a recipient of an islet transplant, the method comprising administering to the recipient of the islet transplant an agent capable of binding to human islet amyloid polypeptide (IAPP) toxic intermediates, wherein binding of the agent to human IAPP toxic intermediates inhibits human IAPP toxic intermediate binding to RAGE and thus reduces islet transplant failure due to human IAPP-mediated toxicity.

21. The method of claim 20, wherein the agent is sRAGE or a functional fragment thereof or an anti-RAGE antibody that inhibits binding of the amyloidogenic polypeptide to RAGE.

22. A method for generating an islet transplant having resistance to islet amyloid polypeptide (IAPP) mediated cytotoxicity, the method comprising incubating pancreatic beta cells with an agent capable of inhibiting binding of human islet amyloid polypeptide (IAPP) toxic intermediates to RAGE or introducing an expression vector that encodes an agent capable of inhibiting binding of IAPP toxic intermediates to RAGE into pancreatic beta cells, thereby generating an islet transplant having resistance to IAPP mediated cytotoxicity.

23. The method of claim 22, wherein the agent is sRAGE or a functional fragment thereof or an anti-RAGE antibody that inhibits binding of the IAPP toxic intermediates to RAGE.

24-29. (canceled)

30. A method of treating a subject with diabetes, the method comprising administering the islet transplant having resistance to islet amyloid polypeptide (IAPP) mediated cytotoxicity of claim 22 to the subject.

31. The method of claim 30, wherein the subject is a human.

32. The method of claim 30, wherein the diabetes is type 1 or type 2 diabetes.
